(19) United States
(12) Patent Application Publication
(10) Pub. No.: US 2005/0112642 A1
(43) Pub. Date: May 26, 2005

(54) Fibroblast growth factor receptors and methods for their use

(75) Inventors: Matthew Sleeman, Suffolk (GB); J. Greg Murison, Auckland (NZ); Zhihui Cao, Auckland (NZ)

Correspondence Address: SPECKMAN LAW GROUP PLLC, 1501 WESTERN AVE, SEATTLE, WA, 98101, US

(73) Assignee: GENESIS RESEARCH AND DEVELOPMENT CORPORATION LIMITED, Auckland (NZ)

(21) Appl. No.: 10/954,094

(22) Filed: Sep. 29, 2004

Related U.S. Application Data
(63) Continuation-in-part of application No. 10/885,225, filed on Jul. 6, 2004.
(63) Continuation-in-part of application No. 10/613,413, filed on Jul. 3, 2003.
Application No. 10/613,413 is a continuation-in-part of application No. 10/157,444, filed on May 28, 2002, now abandoned.
(60) Provisional application No. 60/484,877, filed on Jul. 3, 2003.
(60) Provisional application No. 60/513,171, filed on Oct. 20, 2003.
(60) Provisional application No. 60/562,155, filed on Apr. 14, 2004.
(60) Provisional application No. 60/570,355, filed on May 12, 2004.

(51) Int. Cl. C12Q 1/68; C07H 21/04; C07K 14/71; C07K 16/28

(52) U.S. Cl. 435/6; 435/69.1; 435/320.1; 435/325; 530/350; 530/388.22; 536/23.5

(57) ABSTRACT

Isolated fibroblast growth factor receptor 5 (FGFR5) polypeptides are provided, together with polynucleotides encoding such polypeptides. Also provided are modulators of FGFR5 gene expression and binding molecules that specifically bind to, and agonize or antagonize, FGFR5 polypeptide function. Binding molecules include antibodies, and functional fragments thereof, as well as scFv and Camelidae heavy chain IgG that specifically bind to FGFR5 thereby modulating the activity of FGFR5. Such binding agents and modulators of FGFR5 gene expression may be employed for the treatment of disorders including: osteopontin-mediated diseases; autoimmune diseases, such as systemic lupus erythematosus; bone disorders such as osteoporosis and osteopetrosis; and cancers, including cellular carcinomas such as hepatocellular carcinomas.

### CROSS-REFERENCE TO RELATED APPLICATIONS

This application is a continuation-in-part of U.S. patent application Ser. No. 10/885,225, filed Jul. 6, 2004, which claims priority to U.S. provisional patent application Nos. 60/484,877, filed Jul. 3, 2003; 60/513,171, filed Oct. 20, 2003; 60/562,155, filed Apr. 4, 2004; and 60/570,355, filed May 12, 2004, This application is also a continuation-in-part of U.S. patent application No. 10/613,413, filed Jul. 3, 2003, which is a continuation-in-part of U.S. patent application Ser. No. 10/157,444, filed May 28, 2002, now abandoned.

### TECHNICAL FIELD OF THE INVENTION

This invention relates to fibroblast growth factor receptor 5 (FGFR5) polypeptides, polynucleotides encoding such polypeptides, modulators of FGFR5 gene expression, binding agents, such as antibodies and other molecules that specifically bind to the inventive polypeptides, and the use of such polynucleotides, polypeptides, modulators and binding agents in therapeutic and diagnostic methods. The present invention further relates to splice variants of FGFR5 that are uniquely expressed in various cell types and associated with diseases such as autoimmune diseases and cancers.

### BACKGROUND OF THE INVENTION

Lymph vessels and nodes are important components of the body's immune system. Lymph nodes are small lymphatic organs that are located in the path of lymph vessels. Large molecules and cells, including foreign substances, enter into the lymphatic vessels and, in circulating through these vessels, pass through the lymph nodes. Here, any foreign substances are concentrated and exposed to lymphocytes. This triggers a cascade of events that constitute an immune response, protecting the body from disorders such as infection and cancer.

Lymph nodes are surrounded by a dense connective tissue network that forms a supporting capsule. This network extends into the body of the lymph node, forming an additional framework of support. Throughout the remainder of the organ, a fine meshwork can be identified that comprises reticular fibres and the reticular cells that produce and surround the fibres. These features provide a support for the main functional cells of the lymphatic system, which are T- and B-lymphocytes. Additional cell types found in lymph nodes include macrophages, follicular dendritic cells and endothelial cells that line the blood vessels servicing the node.

The cells within lymph nodes communicate with each other in order to defend the body against foreign substances. When a foreign substance, or antigen, is present, it is detected by macrophages and follicular dendritic cells that take up and process the antigen, and display parts of it on their cell surface. These cell surface antigens are then presented to T- and B-lymphocytes, causing them to proliferate and differentiate into activated T-lymphocytes and plasma cells, respectively. These cells are released into the circulation in order to seek out and destroy antigen. Some T- and B-lymphocytes will also differentiate into memory cells. Should these cells come across the same antigen at a later date, the immune response will be more rapid.

Once activated T- and B-lymphocytes are released into the circulation, they can perform a variety of functions that lead to the eventual destruction of antigen. Activated T-lymphocytes can differentiate into cytotoxic lymphocytes (also known as killer T-cells) which recognise other cells that have foreign antigens on their surface and kill the cell by causing them to lyse. Activated T-lymphocytes can also differentiate into helper T-cells which will then secrete proteins in order to stimulate B-lymphocytes, and other T-lymphocytes, to respond to antigens. In addition, activated T-lymphocytes can differentiate into suppressor T-cells which secrete factors that suppress the activity of B-lymphocytes. Activated B-lymphocytes differentiate into plasma cells, which synthesize and secrete antibodies that bind to foreign antigens. The antibody-antigen complex is then detected and destroyed by macrophages, or by a group of blood constituents known as complement.

Lymph nodes can be dissociated and the resulting cells grown in culture. Cells that adhere to the tissue culture dishes can be maintained for some length of time and are known as stromal cells. The cultured cells are a heterogeneous population and can be made up of most cells residing within lymph nodes, such as reticular cells, follicular dendritic cells, macrophages and endothelial cells. It is well known that bone marrow stromal cells play a critical role in homing, growth and differentiation of hematopoietic progenitor cells. Proteins produced by stromal cells are necessary for the maintenance of plasma cells in vitro. Furthermore, stromal cells are known to secrete factors and present membrane-bound receptors that are necessary for the survival of lymphoma cells.

An autosomal recessive mutation, designated flaky skin (fsn -/-), has been described in the inbred A/J mouse strain (The Jackson Laboratory, Bar Harbour, Me.). The mice have a skin disorder similar to psoriasis in humans. Psoriasis is a common disease affecting 2% of the population, which is characterised by a chronic inflammation associated with thickening and scaling of the skin. Histology of skin lesions shows increased proliferation of the cells in the epidermis, the uppermost layer of skin, together with the abnormal presence of inflammatory cells, including lymphocytes, in the dermis, the layer of skin below the epidermis. While the cause of the disease is unclear, psoriasis is associated with a disturbance of the immune system involving T lymphocytes. The disease occurs more frequently in family members, indicating the involvement of a genetic factor as well. Mice with the fsn gene mutation have not only a psoriatic-like skin disease but also other abnormalities involving cells of the immune and hematopoietic system. These mice have markedly increased numbers of lymphocytes associated with enlarged lymphoid organs, including the spleen and lymph nodes. In addition, their livers are enlarged, and the mice are anaemic. Genes and proteins expressed in abnormal lymph nodes of fsn-/- mice may thus influence the development or function of cells of the immune and hematopoietic system, the response of these cells in inflammatory disorders, and the responses of skin and other connective tissue cells to inflammatory signals.

There is a need in the art to identify genes encoding proteins that function to modulate all cells of the immune system. These proteins from normal or abnormal lymph nodes may be useful in modifying the immune responses to tumour cells or infectious agents such as bacteria, viruses, protozoa and worms. Such proteins may also be useful in the treatment of disorders where the immune system initiates unfavorable reactions to the body, including Type I hypersensitivity reactions (such as hay fever, eczema, allergic rhinitis and asthma), and Type II hypersensitivity reactions (such as transfusion reactions and haemolytic disease of newborns). Other unfavorable reactions are initiated during Type III reactions, which are due to immune complexes forming in infected organs during persistent infection or in the lungs following repeated inhalation of materials from moulds, plants or animals, and in Type IV reactions in diseases such as leprosy, schistosomiasis and dermatitis.

Novel proteins of the immune system may also be useful in treating autoimmune diseases where the body recognises itself as foreign. Examples of such diseases include rheumatoid arthritis, Addison's disease, ulcerative colitis, dermatomyositis and lupus. Such proteins may also be useful during tissue transplantation, where the body will often recognise the transplanted tissue as foreign and attempt to kill it, and also in bone marrow transplantation when there is a high risk of graft-versus-host disease in which the transplanted cells attack their host cells, often causing death.

There thus remains a need in the art for the identification and isolation of genes encoding proteins expressed in cells of the immune system for use in the development of therapeutic agents for the treatment of disorders including those associated with the immune system.

### SUMMARY OF THE INVENTION

The present invention is based upon the identification and isolation of fibroblast growth factor receptor 5 (FGFR5) polypeptides and polynucleotides expressed in lymph node stromal cells of fsn -/- mice, and of human homologues of such polypeptides and polynucleotides. Isolated FGFR5 polypeptides and polynucleotides encoding such FGFR5 polypeptides are provided, together with splice variants thereof of such polynucleotides, expression vectors and host cells comprising the inventive polynucleotides. In addition, the present invention provides modulators of FGFR5 gene expression, and binding agents that specifically bind to, and modulate the activity of, FGFR5 polypeptides, together with compositions comprising such polynucleotides, polypeptides, modulators and/or agents, and methods employing such compositions.

In specific embodiments, the isolated polynucleotides of the present invention comprise a nucleotide sequence selected from the group consisting of: (a) SEQ ID NO: 1-4, 9, 144 and 154; (b) complements of sequences provided in SEQ ID NO: 1-4, 9, 144 and 154; (c) reverse complements of sequences provided in SEQ ID NO: 1-4, 9, 144 and 154; (d) reverse sequences of sequences provided in SEQ ID NO: 1-4, 9, 144 and 154; and (e) sequences having at least 75%, 90% or 95% identity to a sequence of (a)-(d). In further embodiments, the inventive polynucleotides comprise a splice variant of the FGFR5 polynucleotides presented in SEQ ID NO: 1-4, 9, 144 and 154. Exemplary splice variants include the polynucleotides presented herein as SEQ ID NO: 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140 and 142. Polynucleotides comprising a sequence that is a complement, reverse complement, reverse sequence or a variant, as defined herein, of such a splice variant sequence are also encompassed by the present invention.

The present invention further provides isolated polypeptides encoded by the inventive polynucleotides. In specific embodiments, such polypeptides comprise an amino acid sequence selected from the group consisting of: (a) sequences provided in SEQ ID NO: 5-8, 13-15, 145 and 153; (b) sequences having at least 75%, 90% or 95% identity to a sequence of SEQ ID NO: 5-8, 13-15, 145 and 153; and (c) functional portions of a sequence of SEQ ID NO: 5-8, 13-15, 145 and 153. Isolated polypeptides encoded by the FGFR5 splice variant polynucleotides disclosed herein are also provided, together with variants of such polypeptides and functional portions thereof. Exemplary polypeptides encoded by the inventive splice variant polynucleotides include the polypeptides presented herein as SEQ ID NO: 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 and 143.

In related embodiments, the present invention provides expression vectors comprising a polynucleotide of the present invention, together with host cells transformed with such vectors.

In another aspect, the present invention provides fusion proteins comprising at least one polypeptide of the present invention.

In a further aspect, the present invention provides binding agents that specifically bind to FGFR5 polypeptides and that are antagonists or agonists of FGFR5 activity. Such binding agents include antibodies and/or other binding molecules, including small molecules and FGFR5 ligands, or antigen-binding fragments thereof, that specifically bind to one or more antigenic epitopes present on one or more of the inventive FGFR5 polypeptides. Antagonists of FGFR5 encompassed by the present invention also include engineered soluble FGFR5 molecules that bind FGFR5 ligand but do not stimulate signalling. The inventive antibodies may be polyclonal antibodies or monoclonal antibodies, and/or may comprise one or more fragments of a monoclonal antibody such as, for example, a Fab fragment or a small chain antibody fragment (scFv). As detailed below, camelid heavy chain antibodies (HCAb) or heavy chain variable domains thereof (V_{HH}), that bind to FGFR5 polypeptides are also encompassed by the present invention.

Binding agents may be agonists of FGFR5 polypeptide function that are, for example, effective in increasing osteopontin gene expression in a population of cells expressing FGFR5 polypeptide when the agonist is contacted with the population of cells. Alternatively, binding agents may be antagonists of FGFR5 polypeptide function that are, for example, effective in decreasing osteopontin gene expression in a population of cells expressing FGFR5 polypeptide when the antagonist is contacted with the population of cells.

In other aspects, the present invention provides modulators of FGFR5 gene expression. Such modulators may be selected from the group consisting of: anti-sense oligonucleotides to FGFR5; FGFR5-specific small interfering RNA molecules (siRNA or RNAi); monomeric soluble FGFR5; and engineered soluble FGFR5 molecules that bind FGFR5 ligand but do not stimulate signaling. In certain embodiments, modulators of FGFR5 gene expression specifically bind to the FGFR5 polynucleotides disclosed herein. Such modulators of FGFR5 gene expression are effective in decreasing FGFR5 gene expression when contacted with a population of cells expressing FGFR5. Modulators of FGFR5 gene expression may also be effective in decreasing osteopontin gene expression when contacted with a population of cells expressing FGFR5.

As detailed below, the inventive polynucleotides, polypeptides, FGFR5 binding agents and modulators of FGFR5 gene expression may be usefully employed in the preparation of therapeutic agents, or compositions, for the treatment of disorders. Disorders that may be effectively treated using the inventive compositions include inflammatory disorders, disorders of the immune system, cancer, sarcoidal and granulomatous disorders, fibroblast growth factor-mediated disorders, viral disorders, and disorders associated with an abnormal (either elevated or reduced) level of osteopontin. Examples of such disorders include: HIV-infection; epithelial, lymphoid, myeloid, stromal, neuronal, breast, hepatocellular, and colon cancers; arthritis; inflammatory bowel disease; and cardiac failure. Examples of disorders associated with elevated levels of osteopontin include: systemic lupus erythematosus (SLE); multiple sclerosis (MS); diabetes; rheumatoid arthritis (RA); sarcoidosis; tuberculosis; kidney stones; atherosclerosis; vasculitis; nephritis; arthritis; and osteoporosis. An exemplary disorder associated with a reduced level of osteopontin is osteopetrosis.

In related embodiments, methods for modulating an immune response or modulating the growth of blood vessels are provided, together with methods for modulating osteopontin levels. In certain embodiments, the inventive methods include down-regulating, for example reducing the effective amount, inactivating, and/or inhibiting, the activity of a FGFR5 polypeptide or a polynucleotide that encodes such a polypeptide. Such methods may include administering a composition comprising a modulator of FGFR5 gene expression, or an antagonist of FGFR5. Antagonists of FGFR5 polypeptide function that may be usefully employed in the treatment of diseases associated with elevated osteopontin expression include: (a) small molecules; (b) antibodies or antigen-binding fragments thereof; (c) small chain antibody fragments (scFv); and (d) camelid heavy chain antibodies (HCAb) or heavy chain variable domains (V_{HH}) thereof.

The above-mentioned and additional features of the present invention, together with the manner of obtaining them, will be best understood by reference to the following more detailed description. All references disclosed herein are hereby incorporated by reference in their entirety as if each was incorporated individually.

### BRIEF DESCRIPTION OF THE DRAWINGS

FIG. 1 is the amino acid sequence of the murine FGF receptor muFGFR5β (SEQ ID NO: 6). Several conserved domains were identified that are involved in the dimerization, ligand binding and activity of the receptor. The signal peptide and transmembrane domain are underlined, and the six cysteines conserved among the FGFR family members are in bold and underlined. Four glycosylation sites are double underlined. Three immunoglobulin domains (Ig loops) were identified (Ig loop 1: residues 40-102; Ig loop 2: residues 161-224; Ig loop 3: residues 257-341), as well as two tyrosine kinase phosphorylation sites (residues 198-201, 325-332), a cAMP- and cGMP-dependent protein kinase phosphorylation site (residues 208-215) and four prenyl group binding sites (CAAX boxes). The phosphorylation sites and CAAX boxes are boxed. A heparin binding domain was identified (residues 150-167; boxed and in bold) and this partially overlaps the CAM binding domain (residues 141-160; italics and underlined).

FIG. 2A shows the induction of genes under the control of the serum response element (SRE). NIH-3T3 SRE cells were stimulated with a titration of FGF-2 in the presence of 10 μg/ml of heparin for 6 hours. Closed circles represent media alone, open squares represent titration of FGF-2. FIG. 2B shows the competition analysis of NIH-3T3 SRE cells treated with a standard dose of FGF-2 plus heparin in the presence of increasing concentrations of FGFR2Fc (closed diamonds), FGFR5βFc (closed squares), FGFR5γFc (closed triangles) and FGF-2 alone (asterisk). The mean and SD were calculated for both experiments from three separate wells and are represented as fold-induction of the reporter gene relative to control.

FIG. 3 illustrates the stimulation of growth of RAW264.10 cells by FGFR5β and FGFR5γ. This stimulation was not observed when FGF-2 and FGFR2 were used as controls. This stimulation was also not induced by the growth medium.

FIG. 4 illustrates that murine and human FGFR5β-Fc and murine FGFR5γ-Fc augment anti-CD3 induced PBMC proliferation. The enhanced proliferation was not observed when FGFR1, 2, 3 or 4-Fc was used.

FIG. 5 illustrates that murine and human FGFR5β-Fc and murine FGFR5γ-Fc induce the growth of adherent PBMC. The proliferation was not observed when FGFR1, 2, 3 or 4-Fc was used.

FIG. 6 shows the amino acid sequence of human FGFR5 provided in SEQ ID NO: 8. Several conserved domains were identified that are involved in the dimerization, ligand binding and activity of the receptor. The signal peptide is underlined, and five of the six cysteines conserved among the FGFR family members are in bold and underlined. Three immunoglobulin domains (Ig loops) were identified (Ig loop 1: residues 44-106; Ig loop 2: residues 165-228; Ig loop 3 (partial): residues 261-324), as well as a tyrosine kinase phosphorylation sites (residues 212-219), a cAMP- and cGMP-dependent protein kinase phosphorylation site (residues 202-205) and four prenyl group binding sites (CAAX boxes). The phosphorylation sites and CAAX boxes are boxed. A heparin-binding domain was identified (residues 154-171; boxed and in bold) and this partially overlaps the CAM binding domain (residues 145-164; italics and underlined).

FIG. 7A-B are bar graphs depicting upregulation of OPN in adherent PBMC (predominantly monocytes; FIG. 7A) and PBMC (FIG. 7B) following stimulation with FGFR2, FGFR5, LPS or media alone for 24 hours. Supernatants were collected for cytokine analysis.

FIG. 8A-B are graphs depicting the effect of FGFR5 on the proliferation of murine bone marrow cells (BMC; FIG. 8A), and non-adherent BMC (FIG. 8B).

FIG. 9 is a graph depicting the effect of FGFR5 on the proliferation of bone marrow stromal cells.

FIG. 10 is a graph depicting the effect of FGFR5 on 6AVS cell proliferation.

FIG. 11 is a bar graph depicting the preferential expansion of pre-B cells where FIG. 11A depicts the percentage of B220⁺ cells in total viable cells and FIG. 11B depicts the percentage of pre/pro-B cells in total viable B cells.

FIG. 12 is a bar graph depicting the effect of FGFR5 on CFU pre-B formation from BMC.

FIGS. 13 and 14 are graphs showing that monomeric FGFR5 does not augment anti-CD3 stimulated proliferation of PBMC.

FIG. 15 is a graph showing that dimerization of FGFR5-Fc to form tetramers augments the ability of FGFR5-Fc to stimulate growth of adherent PBMC.

FIG. 16 is a graph showing that dimerized monomeric FGFR5 augments the growth of anti-CD3 induced PBMC proliferation in a similar manner as the dimeric FGFR5-Fc fusion protein.

FIG. 17 is a graph showing that dimerized FGFR5-Fc (i.e. tetrameric FGFR5-Fc) augments the anti-CD3 induced growth of human PBMC.

FIGS. 18 and 19 are graphs showing that the FGFR5-specific monoclonal antibody enhances the activity of the monomeric FGFR5 and dimeric FGFR5-Fc fusion protein in the PBMC adherence assay.

FIG. 20 is a graph showing that FGFR5 binds to a heparin Hi-Trap affinity column (Amersham Pharmacia Biotech; Piscataway, N.J.) and is eluted with a salt gradient with a peak at ^{˜}1 M NaCl.

FIG. 21 is a graph showing that heparin inhibits the function of FGFR5 at a concentration of 5 μg/ml thereby blocking the ligand binding portion of FGFR5.

FIG. 22 is a line graph showing that heparin inhibits the FGFR5β-Fc mediated growth of murine bone marrow cells. Murine bone marrow cells were cultured in 96 well microwell plates as described in Example 16 with 20 nM of FGFR5β-Fc and heparin sulphate was titrated into the culture wells at the indicated doses. The cells were cultured for 3 days, pulsed with ³H-TdR for the final 16 hrs of culture. The cells were harvested and the level of proliferation determined by standard liquid scintillation counting.

FIG. 23 is a bar graph demonstrating FGFR5-related changes in the frequency of B-cell subsets in the bone marrow following in vivo intravenous administration of FGFR5-Fc. FGFR5-Fc induced a statistically significant increase in the percentage of pre-B cells (B220⁺CD25⁺) in the bone marrow whereas there was little effect on the immature B cells (B220⁺IgM⁺). The results shown are representative of 2 experiments that yielded similar results.

FIG. 24 shows the average number of cells per lymph node from mice treated with either PBS, FGFR2-Fc or FGFR5γ-Fc on days 1, 2 and 3 after treatment.

FIG. 25 shows the number of B cells (CD19+) and activated B cells (CD19+CD69+) in individual lymph nodes from mice treated with PBS, FGFR2-Fc or FGFR5γ-Fc by subcutaneous footpad injections 1, 2 and 3 days after treatment.

FIG. 26 shows the frequency of B cells (CD19+) and activated B cells (CD19+CD69+) in individual lymph nodes from mice treated with PBS, FGFR2-Fc or FGFR5γ-Fc by subcutaneous footpad injections 1, 2 and 3 days after treatment.

FIG. 27 shows the number of T cells (CD3+) and activated T cells (CD3+CD69+) in individual lymph nodes from mice treated with PBS, FGFR2-Fc or FGFR5γ-Fc by subcutaneous footpad injections 1, 2 and 3 days after treatment.

FIG. 28 shows that the frequency of T cells (CD3+) and activated T cells (CD3+CD69+) in individual lymph nodes from mice treated with PBS, FGFR2-Fc or FGFR5γ-Fc by subcutaneous footpad injections 1, 2 and 3 days after treatment.

FIGS. 29A and B shows the effects of i.p. FGFR5β administration on spleen B cells after 3 injections on odd days from animals euthanized 7 days after the beginning of treatment. FIG. 29A shows the B cell frequency for mice treated with either FGFR5β or FGFR2, as determined using flow cytometry. Values are the mean±SD for 4 mice from a representative experiment, p<0.05 (Student t test). FIG. 29B shows the level of spontaneous proliferation of splenocytes in mice treated with either FGFR5β or FGFR2. Spleen cells from FGFR5β- or FGFR2-treated mice were cultured for 24 h in triplicates in 96-well plates in the presence of 0.25 μCi ³H-thymidine, and proliferation was measured by radioactive ³H-thymidine uptake. Data shown represent mean±SD of triplicate wells from a representative experiment, p<0.001.

FIGS. 30A and B show the effects of i.p. FGFR5β administration on the draining lymph node, posterior mediastinal lymph node. FIG. 30A shows photographs of the lymph nodes from mice treated with either FGFR5 or FGFR2. FIG. 30B shows the frequency of B cells in mice treated with either FGFR5 or FGFR2, as determined using flow cytometry. Values are the mean±SD for 4 mice from a representative experiment, p<0.05 (Student t test).

FIGS. 31A and B show the effects of i.p. administration of FGFR5β or FGFR2 on peritoneal B cell frequency as determined by flow cytometry analysis, with FIG. 31A showing B cell frequency, and FIG. 31B showing B1a cell frequency. Values are the mean±SD for 4 mice from a representative experiment, p<0.01 (Student t test).

FIG. 32 shows the effect of i.p. FGFR5β administration in mice on spleen weight after 3 weeks of treatment. Data are reported as mean weight±SD. n=4 per treatment group; n=2 in untreated group of mice at same age, * p<0.05 (Student t test).

FIGS. 33A and B shows the phenotypic analysis of 2 day and 5 day cultured spleen cells isolated from FGFR5β or FGFR2-treated mice. Cultures of splenocytes isolated from individual mice were pooled and analyzed using flow cytometry. FIG. 33A shows B and T lymphocyte frequency; FIG. 33B shows the percentage of activated cells from each lineage, eg % CD69⁺ CD19⁺/%CD19⁺ cells×100%.

FIG. 34 shows the effect of supernatant collected from 2 day and 5 day cultured spleen cells isolated from FGFR5β-treated mice on the proliferation of splenocytes freshly isolated from untreated mice. Splenocyte cells from untreated mice were cultured for 3 days in triplicates in 96-well plates in the presence of supernatant. Cells were pulsed with 0.25 μCi ³H-thymidine in the last 16 hrs and proliferation was measured by radioactive uptake. Data shown represent mean±SD of the triplicate wells.

FIG. 35 shows the levels of cytokine production in the supernatants of 2 day and 5 day cultured spleen cells isolated from FGFR5β or FGFR2-treated mice. The levels of cytokines were measured using a TH1/TH2 cytokine CBA kit. Data shown represent mean±SD from the cultures of splenocytes isolated from individual mice.

FIG. 36 shows phenotypic analysis of 2-week cultured spleen cells isolated from FGFR5β-treated mice. Cells from cultures of splenocytes isolated from individual mice were pooled and analyzed using flow cytometry.

FIG. 37 shows the increased Ig in sera of FGFR5β-treated mice as determined by ELISA. Data are reported as mean±SD. n=4 in FGFR5β- or FGFR2-treated group; n=2 in untreated group of mice at the same age; n=10 in NZB/W F1 mice.

FIGS. 38A-C shows the quantities of IgG1, IgE and IgG2a, respectively, in sera of FGFR5β-treated mice as determined by ELISA. Data are presented as mean±SD. n=4 in the FGFR5β- or FGFR2-treated groups; n=2 in untreated mice of the same age; n=10 in NZB/W F1 mice.

FIG. 39 shows the increase in serum autoantibody in mice following administration of FGFR5β. Sera from FGFR5β- or FGFR2-treated mice were analyzed for anti-dsDNA using an ELISA assay. Data shown represent mean±SD. n=4 in the FGFR5β or FGFR2-treated groups; n=2 in untreated mice at the same age. A pooled serum from ten NZB/W F1 mice was used as a positive control, and data are reported as mean±SD of triplicate wells.

FIGS. 40A and B show the determination of anti-human Fc and serum anti-FGFR5β activities, respectively, in FGFR5β and FGFR2-treated mice using an ELISA assay. Data shown represent mean±SD from 4 mice in each treatment group.

FIG. 41 shows the effect of FGFR5β on osteoclast formation in mouse bone marrow cultures. Murine bone marrow cells were cultured in the presence or absence of RANKL (50 ng/ml) and M-CSF (50 ng/ml), FGFR5 (5 nM) or FGFR2 (5 nM) for 7 days. The medium was changed every 3 days and fresh cytokines/proteins were added. The cells were fixed and the number of TRAP⁺ cells containing more than three nuclei was quantitated. Values are the mean±SD for two experiments per group

FIGS. 42A-D are photomicrographs demonstrating the effect of FGFR5β administration on TRAP⁺ multinucleated osteoclast formation of mouse bone marrow cells. FIG. 42A shows media control (untreated) cultures; FIG. 42B shows FGFR2 (5 nM)-treated cultures; FIG. 42C shows FGFR5β (5 nM)-treated cultures and FIG. 42D shows cultures treated with RANKL (50 ng/ml) and M-CSF (50 ng/ml). (400× magnification).

FIG. 43 shows FGFR5 gene expression in Zebrafish as determined by in situ hybridization. FIG. 43A: 24 hour post fertilization (hpf). H, head; YS, yolk sac. FIG. 43B: 48 hpf. F, developing fin. FIG. 43C: 5 days post fertilization (dpf). F, fin. Arrows show the positive staining, mRNA expression of FGFR5.

### DETAILED DESCRIPTION OF THE INVENTION

The present invention provides isolated polynucleotides that encode a member of the fibroblast growth factor receptor family referred to as FGFR5 and isolated polypeptides encoded by such polynucleotides, together with modulators of FGFR5 gene expression, and binding agents, such as antibodies and other molecules that specifically bind to the inventive polypeptides. Binding agents of the present invention encompass agonists and/or antagonists of FGFR5 activity. Specific binding agents include antibodies and functional fragments thereof, as well as scFv and Camelidae heavy chain IgG that specifically bind to FGFR5 polypeptides thereby modulating the activity of FGFR5.

As detailed below, FGFR5 has been shown to modulate immune responses and is a potent stimulator of osteopontin expression. Antagonists of FGFR5 may thus be employed in the treatment of disorders associated with, or characterized by, an elevated level of osteopontin. As used herein, the term “elevated level” refers to a level that is higher than the average normal level for a specific patient population. The inventive methods may thus be employed in the treatment of disorders characterized by an abnormal or excessive level of osteopontin compared to levels seen in a normal healthy population. Similarly, FGFR5 and agonists of FGFR5 may be employed in the treatment of disorders characterized by a reduced level of osteopontin.

Osteopontin has been linked with a number of pathophysiological states including a variety of tumors; autoimmune diseases such as multiple sclerosis (MS), systemic lupus erythematosus (SLE), diabetes and rheumatoid arthritis; bone disorders including osteoporosis and osteopetrosis; cancers, including cellular carcinomas such as hepatocellular carcinomas; granulomatous inflammation such as sarcoidosis and tuberculosis; and pathological calcifications such as kidney stones and atherosclerosis. SLE is an autoimmune disorder that affects 24 out of 100,000 individuals in the USA. Afflicted individuals usually develop nephritis, arthritis and dermatitis. Auto-antibody production, complement activation, immune complex deposition, Fc receptor ligation and leukocyte infiltration of the target organs are among the immunopathogenic events.

The term “polynucleotide(s),” as used herein, means a single or double-stranded polymer of deoxyribonucleotide or ribonucleotide bases and includes DNA and corresponding RNA molecules, including HnRNA and mRNA molecules, both sense and anti-sense strands, and comprehends cDNA, genomic DNA and recombinant DNA, as well as wholly or partially synthesized polynucleotides. An HnRNA molecule contains introns and corresponds to a DNA molecule in a generally one-to-one manner. An mRNA molecule corresponds to an HnRNA and DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments. Anti-sense polynucleotides and techniques involving anti-sense polynucleotides are well known in the art and are described, for example, in Robinson-Benion et al., Methods in Enzymol. 254: 363-375, 1995 and Kawasaki et al., Artific. Organs 20: 836-848, 1996.

In specific embodiments, the isolated polynucleotides of the present invention comprise a polynucleotide sequence selected from the group consisting of: sequences provided in SEQ ID NO: 1-4, 9, 144 and 145; and splice variants of a sequence of SEQ ID NO: 1-4, 9, 144 and 145. Exemplary splice variants are presented herein as SEQ ID NO: 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140 and 142. Complements of such isolated polynucleotides, reverse complements of such isolated polynucleotides and reverse sequences of such isolated polynucleotides are also provided, together with polynucleotides comprising at least a specified number of contiguous residues (x-mers) of any of the above-mentioned polynucleotides, extended sequences corresponding to any of the above polynucleotides, antisense sequences corresponding to any of the above polynucleotides, and variants of any of the above polynucleotides, as that term is described in this specification.

The definitions of the terms “complement”, “reverse complement” and “reverse sequence”, as used herein, are best illustrated by the following example. For the sequence 5′ AGGACC 3′, the complement, reverse complement and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

Preferably, sequences that are complements of a specifically recited polynucleotide sequence are complementary over the entire length of the specific polynucleotide sequence.

Some of the polynucleotides of the present invention may be “partial” sequences, in that they do not represent a full length gene encoding a full length polypeptide. Such partial sequences may be extended by analyzing and sequencing various DNA libraries using primers and/or probes and well known hybridization and/or PCR techniques. Partial sequences may be extended until an open reading frame encoding a polypeptide, a full length polynucleotide and/or gene capable of expressing a polypeptide, or another useful portion of the genome is identified. Such extended sequences, including full length polynucleotides and genes, are described as “corresponding to” a sequence identified as one of the sequences of SEQ ID NO: 1-4, 9, 144 and 145, or a variant thereof, or a portion of one of the sequences of SEQ ID NO: 1-4, 9, 144 and 145, or a variant thereof, when the extended polynucleotide comprises an identified sequence or its variant, or an identified contiguous portion (x-mer) of one of the sequences of SEQ ID NO: 1-4, 9, 144 and 145, or a variant thereof. Such extended polynucleotides may have a length of from about 50 to about 4,000 nucleic acids or base pairs, and preferably have a length of less than about 4,000 nucleic acids or base pairs, more preferably yet a length of less than about 3,000 nucleic acids or base pairs, more preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

Similarly, RNA sequences, reverse sequences, complementary sequences, antisense sequences, and the like, corresponding to the polynucleotides of the present invention, may be routinely ascertained and obtained using the cDNA sequences identified as SEQ ID NO: 1-4, 9, 144 and 145, and/or the splice variant sequences of SEQ ID NO: 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140 and 142.

The polynucleotides identified as SEQ ID NO: 1-4, 9, 144 and 145 contain open reading frames (“ORFs”), or partial open reading frames, encoding polypeptides or functional portions of polypeptides. Open reading frames may be identified using techniques that are well known in the art. These techniques include, for example, analysis for the location of known start and stop codons, most likely reading frame identification based on codon frequencies, etc. Open reading frames and portions of open reading frames may be identified in the polynucleotides of the present invention. Suitable tools and software for ORF analysis are well known in the art and include, for example, GeneWise, available from The Sanger Center, Wellcome Trust Genome Campus, Hinxton, Cambridge, CB10 ISA, United Kingdom; Diogenes, available from Computational Biology Centers, University of Minnesota, Academic Health Center, UMHG Box 43 Minneapolis Minn. 55455; and GRAIL, available from the Informatics Group, Oak Ridge National Laboratories, Oak Ridge, Tennessee Tenn. Once a partial open reading frame is identified, the polynucleotide may be extended in the area of the partial open reading frame using techniques that are well known in the art until the polynucleotide for the full open reading frame is identified. Thus, open reading frames encoding polypeptides and/or functional portions of polypeptides may be identified using the polynucleotides of the present invention.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs, or expression vectors, may be introduced into a host cell to express the polypeptide encoded by the open reading frame. Suitable host cells may include various prokaryotic and eukaryotic cells, including plant cells, mammalian cells, bacterial cells, algae and the like.

In another aspect, the present invention provides isolated polypeptides encoded, or partially encoded, by the above polynucleotides. The term “polypeptide”, as used herein, encompasses amino acid chains of any length including full length proteins, wherein amino acid residues are linked by covalent peptide bonds. Polypeptides of the present invention may be naturally purified products, or may be produced partially or wholly using recombinant techniques. Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

The term “polypeptide encoded by a polynucleotide” as used herein, includes polypeptides encoded by a nucleotide sequence which includes a partial isolated DNA sequence of the present invention. In specific embodiments, the inventive polypeptides comprise an amino acid sequence selected from the group consisting of sequences provided in SEQ ID NO: 5-8, 13-15, 145, 153 and variants of such sequences. Isolated polypeptide that comprise an amino acid sequence encoded by a splice variant of one of the FGFR5 polynucleotides presented herein are also provided. Examples of amino acid sequences encoded by FGFR5 splice variants include those provided in SEQ ID NO: 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 and 143.

Polypeptides encoded by the polynucleotides of the present invention may be expressed and used in various assays to determine their biological activity. Such polypeptides may be used to raise antibodies, to isolate corresponding interacting proteins or other compounds, and to quantitatively determine levels of interacting proteins or other compounds.

All of the polynucleotides and polypeptides described herein are isolated and purified, as those terms are commonly used in the art. Preferably, the polypeptides and polynucleotides are at least about 80% pure, more preferably at least about 90% pure, and most preferably at least about 99% pure.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 75%, more preferably at least 80%, more preferably yet at least 90%, and most preferably at least 95% or 98% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100.

Polynucleotide and polypeptide sequences having a specified percentage identity to a polynucleotide or polypeptide identified in one of SEQ ID NO: 1-9, 13-145, 153 and 154, share a high degree of similarity in their primary structure. In addition to a specified percentage identity to a polynucleotide or polypeptide of the present invention, variant polynucleotides and polypeptides preferably have additional structural and/or functional features in common with a polynucleotide or polypeptide of the present invention. Polynucleotides having a specified degree of identity to, or capable of hybridizing to, a polynucleotide of the present invention preferably additionally have at least one of the following features: (1) they contain an open reading frame, or partial open reading frame, encoding a polypeptide, or a functional portion of a polypeptide, having substantially the same functional properties as the polypeptide, or functional portion thereof, encoded by a polynucleotide in a recited SEQ ID NO; or (2) they contain identifiable domains in common.

Polynucleotide or polypeptide sequences may be aligned, and percentages of identical nucleotides or amino acids in a specified region may be determined against another polynucleotide or polypeptide, using computer algorithms that are publicly available. The BLASTN and FASTA algorithms, set to the default parameters described in the documentation and distributed with the algorithm, may be used for aligning and identifying the similarity of polynucleotide sequences. The alignment and similarity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia by contacting the Assistant Provost for Research, University of Virginia, PO Box 9025, Charlottesville, Va. 22906-9025. The BLASTN software is available from the National Centre for Biotechnology Information (NCBI), National Library of Medicine, Building 38A, Room 8N805, Bethesda, Md. 20894. The BLASTN algorithm Version 2.0.11 [Jan. 20, 2000] set to the default parameters described in the documentation and distributed with the algorithm, is preferred for use in the determination of polynucleotide variants according to the present invention. The use of the BLAST family of algorithms, including BLASTN, BLASTP and BLASTX, is described in the publication of Altschul et al., “Gapped BLAST and PSI-BLAST: a new generation of protein database search programs,” Nucleic Acids Res. 25:3389-3402, 1997.

The following running parameters are preferred for determination of alignments and similarities using BLASTN that contribute to the E values and percentage identity for polynucleotides: Unix running command with the following default parameters: blastall -p blastn -d embldb -e 10 -G 0 -E 0 -r 1 -v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (BLASTN only) [Integer]; -v Number of one-line descriptions (V) [Integer]; -b Number of alignments to show (B) [Integer]; -i Query File [File In]; -o BLAST report Output File [File Out] Optional.

The following running parameters are preferred for determination of alignments and similarities using BLASTP that contribute to the E values and percentage identity of polypeptide sequences: blastall -p blastp -d swissprotdb -e 10 -G 0 -E 0 -v 30 -b 30 -i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

As noted above, the percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. By way of example, a queried polynucleotide having 220 nucleic acids has a hit to a polynucleotide sequence in the EMBL database having 520 nucleic acids over a stretch of 23 nucleotides in the alignment produced by the BLASTN algorithm using the default parameters. The 23-nucleotide hit includes 21 identical nucleotides, one gap and one different nucleotide. The percentage identity of the queried polynucleotide to the hit in the EMBL database is thus 21/220 times 100, or 9.5%. The percentage identity of polypeptide sequences may be determined in a similar fashion.

The BLASTN and BLASTX algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleotides or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being related to the polynucleotide or polypeptide of the present invention, measured as having an E value of 0.01 or less using the BLASTN or BLASTX algorithms set at the default parameters. According to a preferred embodiment, a variant polynucleotide is a sequence having the same number or fewer nucleic acids than a polynucleotide of the present invention that has at least a 99% probability of being related to the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being related as the polypeptide of the present invention, measured as having an E value of 0.01 or less using the BLASTP algorithm set at the default parameters.

In an alternative embodiment, variant polynucleotides are sequences that hybridize to a polynucleotide of the present invention under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents, and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity to a polypeptide encoded by a polynucleotide of the present invention. Thus, polynucleotides comprising sequences that differ from the polynucleotide sequences recited in SEQ ID NO: 1-4, 9, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144 and/or 154, or complements, reverse sequences, or reverse complements of those sequences, as a result of conservative substitutions are contemplated by and encompassed within the present invention.

Additionally, polynucleotides comprising sequences that differ from the polynucleotide sequences recited in SEQ ID NO: 1-4, 9, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144 and/or 154, or complements, reverse complements or reverse sequences thereof, as a result of deletions and/or insertions totaling less than 10% of the total sequence length are also contemplated by and encompassed within the present invention.

Similarly, polypeptides comprising sequences that differ from the polypeptide sequences recited in SEQ ID NO: 5-8, 13-15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145 and 153 as a result of amino acid substitutions, insertions, and/or deletions totaling less than 10% of the total sequence length are contemplated by and encompassed within the present invention, provided the variant polypeptide has functional properties which are substantially the same as, or substantially similar to, those of a polypeptide comprising a sequence of SEQ ID NO: 5-8, 13-15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145 and 153.

Polynucleotides of the present invention also comprehend polynucleotides comprising at least a specified number of contiguous residues (x-mers) of any of the polynucleotides identified as SEQ ID NO: 1-4, 9, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142,, 144 and 154 complements, reverse sequences, and reverse complements of such sequences, and their variants. Similarly, polypeptides of the present invention comprehend polypeptides comprising at least a specified number of contiguous residues (x-mers) of any of the polypeptides identified as SEQ ID NO: 5-8, 13-15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145 and 154, and their variants. As used herein, the term “x-mer,” with reference to a specific value of “x,” refers to a sequence comprising at least a specified number (“x”) of contiguous residues of any of the polynucleotides or polypeptides identified herein. According to preferred embodiments, the value of x is preferably at least 20, more preferably at least 40, more preferably yet at least 60, and most preferably at least 80. Thus, polynucleotides and polypeptides of the present invention comprise a 20-mer, a 40-mer, a 60-mer, an 80-mer, a 100-mer, a 120-mer, a 150-mer, a 180-mer, a 220-mer, a 250-mer, a 300-mer, 400-mer, 500-mer or 600-mer of a polynucleotide or polypeptide identified as SEQ ID NO: 1-9, 13-145, 153, 154, and variants thereof.

The inventive polynucleotides may be isolated by high throughput sequencing of cDNA libraries prepared from lymph node stromal cells of fsn -/- mice as described below in Example 1. Alternatively, oligonucleotide probes based on the polynucleotide sequences provided herein can be synthesized and used to identify positive clones in either cDNA or genomic DNA libraries from lymph node stromal cells of fsn -/- mice by means of hybridization or polymerase chain reaction (PCR) techniques. Probes can be shorter than the sequences provided herein but should be at least about 10, preferably at least about 15 and most preferably at least about 20 nucleotides in length. Hybridization and PCR techniques suitable for use with such oligonucleotide probes are well known in the art (see, for example, Mullis et al., Cold Spring Harbor Symp. Quant. Biol., 51:263, 1987; Erlich ed., PCR Technology, Stockton Press, NY, 1989; Sambrook et al., Molecular cloning—a laboratory manual, Cold Spring Harbor Laboratory Press, Cold Spring Harbor, N.Y., 1989). Positive clones may be analyzed by restriction enzyme digestion, DNA sequencing or the like.

The polynucleotides of the present invention may alternatively be synthesized using techniques that are well known in the art. The polynucleotides may be synthesized, for example, using automated oligonucleotide synthesizers (e.g., Beckman Oligo 1000M DNA Synthesizer) to obtain polynucleotide segments of up to 50 or more nucleic acids. A plurality of such polynucleotide segments may then be ligated using standard DNA manipulation techniques that are well known in the art of molecular biology. One conventional and exemplary polynucleotide synthesis technique involves synthesis of a single stranded polynucleotide segment having, for example, 80 nucleic acids, and hybridizing that segment to a synthesized complementary 85 nucleic acid segment to produce a 5 nucleotide overhang. The next segment may then be synthesized in a similar fashion, with a 5 nucleotide overhang on the opposite strand. The “sticky” ends ensure proper ligation when the two portions are hybridized. In this way, a complete polynucleotide of the present invention may be synthesized entirely in vitro.

Polypeptides of the present invention may be produced recombinantly by inserting a DNA sequence that encodes the polypeptide into an expression vector and expressing the polypeptide in an appropriate host. Any of a variety of expression vectors known to those of ordinary skill in the art may be employed. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, insect, yeast or a mammalian cell line such as COS or CHO. The DNA sequences expressed in this manner may encode naturally occurring polypeptides, portions of naturally occurring polypeptides, or other variants thereof.

In a related aspect, polypeptides are provided that comprise at least a functional portion of a polypeptide having an amino acid sequence selected from the group consisting of sequences provided in SEQ ID NO: 5-8, 13-15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 153, and variants thereof. As used herein, the “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity. Such functional portions generally comprise at least about 5 amino acid residues, more preferably at least about 10, and most preferably at least about 20 amino acid residues. Functional portions of the inventive polypeptides may be identified by first preparing fragments of the polypeptide, by either chemical or enzymatic digestion of the polypeptide or mutation analysis of the polynucleotide that encodes for the polypeptide, and subsequently expressing the resultant mutant polypeptides. The polypeptide fragments or mutant polypeptides are then tested to determine which portions retain the biological activity of the full-length polypeptide. Portions and other variants of the inventive polypeptides may be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain (Merrifield, J. Am. Chem. Soc. 85:2149-2154, 1963). Equipment for automated synthesis of polypeptides is available from suppliers such as Perkin Elmer/Applied BioSystems, Inc. (Foster City, Calif.), and may be operated according to the manufacturer's instructions. Variants of a native polypeptide may be prepared using standard mutagenesis techniques, such as oligonucleotide-directed site-specific mutagenesis (see, for example, Kunkel, Proc. Natl. Acad. Sci. USA 82:488-492, 1985). Sections of DNA sequence may also be removed using standard techniques to permit preparation of truncated polypeptides.

The present invention also provides fusion proteins comprising a first and a second inventive polypeptide or, alternatively, a polypeptide of the present invention and a known polypeptide, together with variants of such fusion proteins. The fusion proteins of the present invention may include a linker peptide between the first and second polypeptides.

A polynucleotide encoding a fusion protein of the present invention is constructed using known recombinant DNA techniques to assemble separate polynucleotides encoding the first and second polypeptides into an appropriate expression vector. The 3′ end of a polynucleotide encoding the first polypeptide is ligated, with or without a peptide linker, to the 5′ end of a DNA sequence polynucleotide encoding the second polypeptide so that the reading frames of the sequences are in phase to permit mRNA translation of the two polynucleotides into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may be from 1 to about 50 amino acids in length. Peptide linker sequences are not required when the first and second polypeptides have non-essential N-terminal amino acid regions that can be used to separate the functional domains and prevent steric interference.

The ligated polynucleotides encoding the fusion proteins are cloned into suitable expression systems using techniques known to those of ordinary skill in the art.

The polynucleotides of the present invention may also be used as markers for tissue, as chromosome markers or tags, in the identification of genetic disorders, and for the design of oligonucleotides for examination of expression patterns using techniques well known in the art, such as the microarray technology available from Affymetrix (Santa Clara, Calif.). Partial polynucleotide sequences disclosed herein may be employed to obtain full length genes by, for example, screening of DNA expression libraries, and to isolate homologous DNA sequences from other species using hybridization probes or PCR primers based on the inventive sequences.

The isolated polynucleotides of the present invention also have utility in genome mapping, in physical mapping, and in positional cloning of genes. As detailed below, the polynucleotide sequences identified as SEQ ID NO: 1-4, 9, 144 and 154 and their variants, may be used to design oligonucleotide probes and primers. Oligonucleotide probes designed using the polynucleotides of the present invention may be used to detect the presence and examine the expression patterns of genes in any organism having sufficiently similar DNA and RNA sequences in their cells using techniques that are well known in the art, such as slot blot DNA hybridization techniques. Oligonucleotide primers designed using the polynucleotides of the present invention may be used for PCR amplifications. Oligonucleotide probes and primers designed using the polynucleotides of the present invention may also be used in connection with various microarray technologies, including the microarray technology of Affymetrix (Santa Clara, Calif.).

As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction. An oligonucleotide probe or primer is described as “corresponding to” a polynucleotide of the present invention, including one of the sequences set out as SEQ ID NO: 1-4, 9, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144 and 154 or a variant thereof, if the oligonucleotide probe or primer, or its complement, is contained within one of the sequences set out as SEQ ID NO: 1-4, 9, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140 142, 144 and 145 or a variant of one of the specified sequences. Oligonucleotide probes and primers of the present invention are substantially complementary to a polynucleotide disclosed herein.

Two single stranded sequences are said to be substantially complementary when the nucleotides of one strand, optimally aligned and compared, with the appropriate nucleotide insertions and/or deletions, pair with at least 80%, preferably at least 90% to 95% and more preferably at least 98% to 100% of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone.

In specific embodiments, the oligonucleotide probes and/or primers comprise at least about 6 contiguous residues, more preferably at least about 10 contiguous residues, and most preferably at least about 20 contiguous residues complementary to a polynucleotide sequence of the present invention. Probes and primers of the present invention may be from about 8 to 100 base pairs in length or, preferably from about 10 to 50 base pairs in length or, more preferably from about 15 to 40 base pairs in length. The probes can be easily selected using procedures well known in the art, taking into account DNA-DNA hybridization stringencies, annealing and melting temperatures, and potential for formation of loops and other factors, which are well known in the art. Tools and software suitable for designing probes and PCR primers are well known in the art and include the software program available from Premier Biosoft International, 3786 Corina Way, Palo Alto, Calif. 94303-4504. Preferred techniques for designing PCR primers are also disclosed in Dieffenbach, C W and Dyksler, G S. PCR Primer: a laboratory manual, CSHL Press: Cold Spring Harbor, N.Y., 1995.

A plurality of oligonucleotide probes or primers corresponding to a polynucleotide of the present invention may be provided in a kit form. Such kits generally comprise multiple DNA or oligonucleotide probes or primers, each probe or primer being specific for a polynucleotide sequence. Kits of the present invention may comprise one or more probes or primers corresponding to a polynucleotide of the present invention, including a polynucleotide sequence identified in SEQ ID NO: 1-4, 9, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140 142, 144 and 154.

In one embodiment useful for high-throughput assays, the oligonucleotide probe kits of the present invention comprise multiple probes in an array format, wherein each probe is immobilized at a predefined, spatially addressable, location on the surface of a solid substrate. Array formats which may be usefully employed in the present invention are disclosed, for example, in U.S. Pat. Nos. 5,412,087 and 5,545,451, and PCT Publication No. WO 95/00450, the disclosures of which are hereby incorporated by reference.

The polypeptides provided by the present invention may additionally be used in assays to determine biological activity, to raise antibodies, to isolate corresponding ligands or receptors, in assays to quantify levels of protein or cognate corresponding ligand or receptor, as anti-inflammatory agents, and in compositions for the treatment of diseases of the immune system.

The present invention further provides methods and compositions for modulating the levels and/or inhibiting the activity of an inventive polypeptide or polynucleotide. As used herein, the term “modulate” or “modulating” includes an increase or a decrease in polynucleotide expression and/or an increase or a decrease in polypeptide function. Thus, the term “modulator” encompasses both “agonists” of protein function and “antagonists” of protein function, wherein the term “agonists” refers to an agent that increases polypeptide function, and the term “antagonist” refers to an agent that decreases polypeptide function.

Methods employing modulators of the present invention include administering a molecule, compound and/or composition selected from the group consisting of: antibodies, antigen-binding fragments thereof, small chain antibody variable domain fragments (scFv), and camelid heavy chain antibody (HCAb) or heavy chain variable domain thereof (V_{HH}) that specifically bind to a polypeptide of the present invention; soluble ligands that bind to an inventive polypeptide; small molecule inhibitors of the inventive polypeptides and/or polynucleotides; anti-sense oligonucleotides to the inventive polynucleotides; small interfering RNA molecules (siRNA or RNAi) that are specific for a polynucleotide or polypeptide of the present invention; and engineered soluble polypeptide molecules that bind a ligand of an inventive polypeptide but do not stimulate signaling.

Small molecule inhibitors of the present invention, which may be either organic or inorganic, preferably have a molecular weight up to about 1500 daltons. Small molecules, can include, but are not limited to, compounds obtained from any commercial source, including Aldrich (1001 West St. Paul Ave., Milwaukee, Wis. 53233), Sigma Chemical (P.O. Box 14508, St. Louis, Mo. 63178), Fluka Chemie Ag (Industriestrasse 25, CH-9471 Buchs, Switzerland (Fluka Chemical Corp. 980 South 2nd Street, Ronkonkoma, N.Y. 11779)), Eastman Chemical Company, Fine Chemicals (P.O. Box 431, Kingsport, Tenn. 37662), Boehringer Mannheim GmbH (Sandhofer Strasse 116, D-68298 Mannheim, Takasago (4 Volvo Drive, Rockleigh, N.J. 07647), SST Corporation (635 Brighton Road, Clifton, N.J. 07012), Ferro (111 West Irene Road, Zachary, La. 70791), Riedel-deHaen Aktiengesellschaft (P.O. Box D-30918, Seelze, Germany), and PPG Industries Inc., Fine Chemicals (One PPG Place, 34th Floor, Pittsburgh, Pa. 15272). Llibraries of small molecule test compounds may be commercially obtained, for example, from Specs and BioSpecs B. V. (Rijswijk, The Netherlands), Chembridge Corporation (San Diego, Calif.), Contract Service Company (Dolgoprudny, Moscow Region, Russia), Comgenex USA Inc. (Princeton, N.J.), Maybridge Chemical Ltd. (Cornwall PL34 OHW, United Kingdom), and Asinex (Moscow, Russia). Furthermore, combinatorial libraries of small molecule test compounds, may be generated as disclosed in Eichler & Houghten, (Mol. Med. Today 1:174-180, 1995); Dolle (Mol. Divers. 2:223-236, 1997); Lam (Anticancer Drug Des. 12:145-167, 1997).

Small molecule inhibitors of the present invention may be identified by: (a) exposing at least one small molecule test compound to a FGFR5 polypeptide of the present invention for a time sufficient to allow binding of the test compound(s) to the polypeptide; (b) removing non-bound test compounds; and (c) determining the presence of the test compound bound to the polypeptide. Alternatively, small molecule inhibitors of the present invention may be identified by: (a) exposing at least one small molecule test compound to a FGFR5 polypeptide of the present invention for a time sufficient to allow binding of the test compound to the polypeptide; (b) removing non-bound compounds; and (c) determining the presence of the compound bound to the polypeptide.

The present invention further provides methods and compositions for reducing the levels and/or inhibiting the activity of an inventive polypeptide or polynucleotide. Such methods include administering a component selected from the group consisting of: antibodies, or antigen-binding fragments thereof, that specifically bind to a polypeptide of the present invention; soluble ligands that bind to an inventive polypeptide; small molecule inhibitors of the inventive polypeptides and/or polynucleotides; anti-sense oligonucleotides to the inventive polynucleotides; small interfering RNA molecules (siRNA or RNAi) that are specific for a polynucleotide or polypeptide of the present invention; and engineered soluble polypeptide molecules that bind a ligand of an inventive polypeptide but do not stimulate signaling.

Modulating the activity of a polypeptide described herein may be accomplished by reducing or inhibiting expression of the polypeptides, which can be achieved by interfering with transcription and/or translation of the corresponding polynucleotide. Polypeptide expression may be inhibited, for example, by introducing anti-sense expression vectors, anti-sense oligodeoxyribonucleotides, anti-sense phosphorothioate oligodeoxyribonucleotides, anti-sense oligoribonucleotides or anti-sense phosphorothioate oligoribonucleotides; or by other means well known in the art. All such anti-sense polynucleotides are referred to collectively herein as “anti-sense oligonucleotides”.

The anti-sense oligonucleotides disclosed herein are sufficiently complementary to the polynucleotide encoding the inventive polypeptide to bind specifically to the polynucleotide. The sequence of an anti-sense oligonucleotide need not be 100% complementary to that of the polynucleotide in order for the anti-sense oligonucleotide to be effective in the inventive methods. Rather an anti-sense oligonucleotide is sufficiently complementary when binding of the anti-sense oligonucleotide to the polynucleotide interferes with the normal function of the polynucleotide to cause a loss of utility, and when non-specific binding of the oligonucleotide to other, non-target, sequences is avoided. The present invention thus encompasses polynucleotides in an anti-sense orientation that inhibit translation of the inventive polypeptides. The design of appropriate anti-sense oligonucleotides is well known in the art. Oligonucleotides that are complementary to the 5′ end of the message, for example the 5′ untranslated sequence up to and including the AUG initiation codon, should work most efficiently at inhibiting translation. However, oligonucleotides complementary to either the 5′- or 3′-non-translated, non-coding, regions of the targeted polynucleotide can be used.

Cell permeation and activity of anti-sense oligonucleotides can be enhanced by appropriate chemical modifications, such as the use of phenoxazine-substituted C-5 propynyl uracil oligonucleotides (Flanagan et al., Nat. Biotechnol. 17:48-52 (1999)) or 2′-O-(2-methoxy) ethyl (2′-MOE)-oligonucleotides (Zhang et al., Nat. Biotechnol. 18:862-867 (2000)). The use of techniques involving anti-sense oligonucleotides is well known in the art and is described, for example, in Robinson-Benion et al., Methods in Enzymol. 254:363-375 (1995) and Kawasaki et al., Artific. Organs 20:836-848 (1996).

Expression of a polypeptide of the present invention may also be specifically suppressed by methods such as RNA interference (RNAi). A review of this technique is found in Science, 288:1370-1372, 2000. Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-transcriptional level and is sequence specific, but suppresses gene expression far more efficiently. Exemplary methods for controlling or modifying gene expression are provided in WO 99/49029, WO 99/53050 and WO01/75164, the disclosures of which are hereby incorporated by reference. In these methods, post-transcriptional gene silencing is brought about by a sequence-specific RNA degradation process which results in the rapid degradation of transcripts of sequence-related genes. Studies have shown that double-stranded RNA may act as a mediator of sequence-specific gene silencing (see, for example, Montgomery and Fire, Trends in Genetics, 14:255-258, 1998). Gene constructs that produce transcripts with self-complementary regions are particularly efficient at gene silencing.

It has been demonstrated that one or more ribonucleases specifically bind to and cleave double-stranded RNA into short fragments. The ribonuclease(s) remains associated with these fragments, which in turn specifically bind to complementary mRNA, i.e. specifically bind to the transcribed mRNA strand for the gene of interest. The mRNA for the gene is also degraded by the ribonuclease(s) into short fragments, thereby obviating translation and expression of the gene. Additionally, an RNA-polymerase may act to facilitate the synthesis of numerous copies of the short fragments, which exponentially increases the efficiency of the system. A unique feature of RNAi is that silencing is not limited to the cells where it is initiated. The gene-silencing effects may be disseminated to other parts of an organism.

The polynucleotides of the present invention may thus be employed to generate gene silencing constructs and/or gene-specific self-complementary, double-stranded RNA sequences that can be delivered by conventional art-known methods. A gene construct may be employed to express the self-complementary RNA sequences. Alternatively, cells are contacted with gene-specific double-stranded RNA molecules, such that the RNA molecules are internalized into the cell cytoplasm to exert a gene silencing effect. The double-stranded RNA must have sufficient homology to the targeted gene to mediate RNAi without affecting expression of non-target genes. The double-stranded DNA is at least 20 nucleotides in length, and is preferably 21-23 nucleotides in length. Preferably, the double-stranded RNA corresponds specifically to a polynucleotide of the present invention. The use of small interfering RNA (siRNA) molecules of 21-23 nucleotides in length to suppress gene expression in mammalian cells is described in WO 01/75164. Tools for designing optimal inhibitory siRNAs include that available from DNAengine Inc. (Seattle, Wash.).

One RNAi technique employs genetic constructs within which sense and anti-sense sequences are placed in regions flanking an intron sequence in proper splicing orientation with donor and acceptor splicing sites. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes.

As used herein, the phrase “contacting a population of cells with a genetic construct, anti-sense oligonucleotide or RNA molecule” includes any means of introducing a nucleic acid molecule into any portion of one or more cells by any method compatible with cell viability and known to those of ordinary skill in the art. The cell or cells may be contacted in vivo, ex vivo, in vitro, or any combination thereof.

For in vivo uses, a genetic construct, anti-sense oligonucleotide or RNA molecule may be administered by various art-recognized procedures. See, e.g., Rolland, Crit. Rev. Therap. Drug Carrier Systems 15:143-198 (1998), and cited references. Both viral and non-viral delivery methods have been used for gene therapy. Useful viral vectors include, for example, adenovirus, adeno-associated virus (AAV), retrovirus, vaccinia virus and avian poxvirus. Improvements have been made in the efficiency of targeting genes to tumor cells with adenoviral vectors, for example, by coupling adenovirus to DNA-polylysine complexes and by strategies that exploit receptor-mediated endocytosis for selective targeting. See, e.g., Curiel et al., Hum. Gene Ther., 3:147-154 (1992); and Cristiano and Curiel, Cancer Gene Ther. 3:49-57 (1996). Non-viral methods for delivering polynucleotides are reviewed in Chang & Seymour, (Eds) Curr. Opin. Mol. Ther., vol. 2 (2000). These methods include contacting cells with naked DNA, cationic liposomes, or polyplexes of polynucleotides with cationic polymers and dendrimers for systemic administration (Chang & Seymour, Ibid.). Liposomes can be modified by incorporation of ligands that recognize cell-surface receptors and allow targeting to specific receptors for uptake by receptor-mediated endocytosis. See, for example, Xu et al., Mol. Genet. Metab., 64:193-197 (1998); and Xu et al., Hum. Gene Ther., 10:2941-2952 (1999).

Tumor-targeting bacteria, such as Salmonella, are potentially useful for delivering genes to tumors following systemic administration (Low et al., Nat. Biotechnol. 17:37-41 (1999)). Bacteria can be engineered ex vivo to penetrate and to deliver DNA with high efficiency into mammalian epithelial cells in vivo and in vitro. See, e.g., Grillot-Courvalin et al., Nat. Biotechnol. 16:862-866 (1998). Degradation-stabilized oligonucleotides may be encapsulated into liposomes and delivered to patients by injection either intravenously or directly into a target site. Alternatively, retroviral or adenoviral vectors, or naked DNA expressing anti-sense RNA for the inventive polypeptides, may be delivered into a patient's cells in vitro or directly into patients in vivo by appropriate routes. Suitable techniques for use in such methods are well known in the art.

The present invention further provides binding agents, such as antibodies, which specifically bind to a polypeptide disclosed herein, or to a portion or variant thereof. A binding agent is said to “specifically bind” to an inventive polypeptide if it reacts at a detectable level with the polypeptide, and does not react detectably with unrelated polypeptides under similar conditions. Any agent that satisfies this requirement may be a binding agent. For example, a binding agent may be a ribosome, with or without a peptide component, an RNA molecule, or a polypeptide. In preferred embodiments, a binding agent is an antibody, an antigen-binding fragment thereof, small chain antibody variable domain fragments (scFv), or camelid heavy chain antibody (HCAb) or heavy chain variable domain thereof (V_{HH}). The ability of a binding agent to specifically bind to a polypeptide can be determined, for example, in an ELISA assay using techniques well known in the art.

An “antigen-binding site,” or “antigen-binding fragment” of an antibody refers to the part of the antibody that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the inventive polypeptide is initially injected into any of a wide variety of mammals (e.g., mice, rats, rabbits, sheep or goats). The polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the inventive polypeptide may then be purified from such antisera by, for example, affinity chromatography using the polypeptide coupled to a suitable solid support.

Monoclonal antibodies specific for an inventive polypeptide may be prepared using the technique of Kohler and Milstein, Eur. J. Immunol. 6:511-519, 1976, and improvements thereto. These methods involve the preparation of immortal cell lines capable of producing antibodies having the desired specificity. Such cell lines may be produced from spleen cells obtained from an animal immunized as described above. The spleen cells are then immortalized by, for example, fusion with a myeloma cell fusion partner, preferably one that is syngeneic with the immunized animal. A variety of fusion techniques well known in the art may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may then be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of molecules are known in the art that comprise antigen-binding sites capable of exhibiting the binding properties of an antibody molecule. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al. Proc. Nat. Acad. Sci. USA 69:2659-2662 (1972); Hochman et al. Biochem 15:2706-2710 (1976); and Ehrlich et al. Biochem 19:4091-4096 (1980)).

The present invention further encompasses humanized antibodies that specifically bind to an inventive polypeptide. A number of humanized antibody molecules comprising an antigen-binding site derived from a non-human immunoglobulin have been described, including chimeric antibodies having rodent V regions and their associated CDRs fused to human constant domains (Winter et al. Nature 349:293-299 (1991); Lobuglio et al. Proc. Nat. Acad. Sci. USA 86:4220-4224 (1989); Shaw et al. J. Immunol. 138:4534-4538 (1987); and Brown et al. Cancer Res. 47:3577-3583 (1987)); rodent CDRs grafted into a human supporting FR prior to fusion with an appropriate human antibody constant domain (Riechmann et al. Nature 332:323-327 (1988); Verhoeyen et al. Science 239:1534-1536 (1988); and Jones et al. Nature 321:522-525 (1986)); and rodent CDRs supported by recombinantly veneered rodent FRs (European Patent Publication No. 519,596, published Dec. 23, 1992). These “humanized” molecules are designed to minimize unwanted immunological responses towards rodent antihuman antibody molecules which limit the duration and effectiveness of therapeutic applications of those moieties in human recipients.

The present invention also encompasses single-chain antibody fragments, including scFv and Camelidae heavy chain antibodies (HCAb) that specifically bind to one of the FGFR5 polypeptides presented as SEQ ID NO: 5-8, 13-15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145 and 153, or a variant thereof.

ScFv comprise an antibody heavy chain variable region (V_{H}) operably linked to an antibody light chain variable region (V_{L}) wherein the heavy chain variable region and the light chain variable region, together or individually, form a binding site for specifically binding an FGFR5 polypeptide presented herein. ScFv may comprise a V_{H} region at the amino-terminal end and a V_{L} region at the carboxy-terminal end. Alternatively, scFv may comprise a V_{L} region at the amino-terminal end and a V_{H} region at the carboxy-terminal end.

ScFv disclosed herein may, optionally, further comprise a polypeptide linker operably linked between the heavy chain variable region and the light chain variable region. Such polypeptide linkers generally comprise between 1 and 50 amino acids. More preferred are polypeptide linkers of at least 2 amino acids. Within other embodiments, however, polypeptide linkers are preferably between 3 and 12 amino acids. An exemplary linker peptide for incorporating between scFv heavy and light chains comprises the 5 amino acid sequence Gly-Gly-Gly-Gly-Ser (SEQ ID NO: 146). Alternative exemplary linker peptides comprise one or more tandem repeats of this sequence to create linkers comprising, for example, the sequences Gly-Gly-Gly-Gly-Ser-Gly-Gly-Gly-Gly-Ser (SEQ ID NO: 147), Gly-Gly-Gly-Gly-Ser-Gly-Gly-Gly-Gly-Ser-Gly-Gly-Gly-Gly-Ser (SEQ ID NO: 148), and Gly-Gly-Gly-Gly-Ser-Gly-Gly-Gly-Gly-Ser-Gly-Gly-Gly-Gly-Ser-Gly-Gly-Gly-Gly-Ser (SEQ ID NO: 149).

Other embodiments of the present invention provide Camelidae heavy chain antibodies (HCAb) that specifically bind to the inventive polypeptides. These heavy chain antibodies are a class of IgG that are devoid of light chains and that are produced by animals of the genus Camelidae (including camels, dromedaries and llamas). Hamers-Casterman et al., Nature 363:446-448 (1993). HCAbs have a molecular weight of ˜95 kDa instead of the ˜160 kDa molecular weight of conventional IgG antibodies. Their binding domains consist only of the heavy-chain variable domains, referred to as V_{HH} to distinguish them from conventional V_{H}. Muyldermans et al., J. Mol. Recognit. 12:131-140 (1999). Since the first constant domain (C_{H}1) is absent (spliced out during mRNA processing due to loss of a splice consensus signal), the variable domain (V_{HH}) is immediately followed by the hinge region, the CH2 and the CH3 domains. Nguyen et al., Mol. Immunol. 36:515-524 (1999); Woolven et al., Immunogenetics 50:98-101 (1999). Although the HCAbs are devoid of light chains, they have an authentic antigen-binding repertoire. The current knowledge about the genetic generation mechanism of HCAbs is reviewed in Nguyen et al. Adv. Immunol 79:261-296 (2001) and Nguyen et al., Immunogenetics 54:39-47 (2002). Sharks, including the nurse shark, display similar antigen receptor-containing single monomeric V-domains. Irving et al., J. Immunol. Methods 248:31-45 (2001); Roux et al., Proc. Natl. Acad. Sci. USA 95:11804 (1998).

V_{HH}S comprise the smallest available intact antigen-binding fragment (˜15 kDa, 118-136 residues). The affinities of V_{HH}S are typically in the nanomolar range and comparable with those of Fab and scFv fragments. In addition, V_{HH}S are highly soluble and more stable than the corresponding derivatives of scFv and Fab fragments. V_{HH}S carry amino acid substitutions that make them more hydrophilic and prevent prolonged interaction with BiP (Immunoglobulin heavy-chain binding protein), which normally binds to the H-chain in the Endoplasmic Reticulum (ER) during folding and assembly, until it is displaced by the L-chain. Because of the V_{HH}S' increased hydrophilicity, secretion from the ER is improved.

Functional V_{HH}S may be obtained from proteolysed HCAb of an immunized camelid, by direct cloning of V_{HH} genes from B-cells of an immunized camelid resulting in recombinant V_{HH}S, or from naïve or synthetic libraries. V_{HH}S with desired antigen specificity may also be obtained through phage display methodology. Using V_{HH}S in phage display is much simpler and more efficient compared to Fabs or scFvs, since only one domain needs to be cloned and expressed to obtain a functional antigen-binding fragment. Muyldermans, Biotechnol. 74:277-302 (2001); Ghahroudi et al., FEBS Lett. 414:521-526 (1997); and van der Linden et al., J. Biotechnol. 80:261-270 (2000).

Alternatively, ribosome display methodology may be employed for the identification and isolation of scFv and/or V_{HH} molecules having the desired binding activity and affinity. Irving et al., J. Immunol. Methods 248:31-45 (2001). Ribosome display and selection has the potential to generate and display large libraries representative of the theoretical optima for naïve repertoires (10¹⁴).

Other embodiments provide V_{HH}-like molecules generated, through the process of camelisation, by modifying non-Camelidae V_{H}S, such as human V_{H}S, to improve their solubility and prevent non-specific binding. This is achieved by replacing residues on the V_{L} side of V_{H}S with V_{HH}-like residues, thereby mimicking the more soluble V_{HH} fragments. Camelised V_{H} fragments, particularly those based on the human framework, are expected to exhibit a greatly reduced immune response when administered in vivo to a patient and, accordingly, are expected to have significant advantages for therapeutic applications. Davies et al., FEBS Lett. 339:285-290 (1994); Davies et al., Protein Eng. 9:531-537 (1996); Tanha et al., J. Biol. Chem. 276:24774-24780 (2001); and Riechmann et al., Immunol. Methods 231:25-38 (1999).

A wide variety of expression systems are available in the art for the production of anti-FGFR5 antibody fragments including Fab fragments, scFv, and V_{HH}S. For example, expression systems of both prokaryotic and eukaryotic origin may be used for the large-scale production of antibody fragments and antibody fusion proteins. Particularly advantageous are expression systems that permit the secretion of large amounts of antibody fragments into the culture medium.

Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins. Yeasts and filamentous fungi are accessible for genetic modifications and the protein of interest may be secreted into the culture medium. In addition, some of the products comply with the GRAS (Generally Regarded as Safe) status in that they do not harbor pyrogens, toxins, or viral inclusions.

Methylotrophic and other yeasts such as Candida boidinii, Hansenula polymorpha, Pichia methanolica, and Pichia pastoris are well known systems for the production of heterologous proteins. High levels of proteins, in milligram to gram quantities, can be obtained and scaling up to fermentation for industrial applications is possible.

The P. pastoris system is used in several industrial-scale production processes. For example, the use of Pichia for the expression of scFv fragments as well as recombinant antibodies and fragments thereof, has been described. Ridder et al., Biotechnology 13:255-260 (1995); Anadrade et al., J. Biochem (Tokyo) 128:891-895 (2000); Pennell et al., Res. Immunol. 149:599-603 (1998). In shake-flask cultures, levels of 250 mg/L to over 1 g/L of scFv or V_{HH} can be achieved. Eldin et al., J. Immunol. Methods 201:67-75 (1997); Freyre et al., J. Biotechnol. 76:157-163 (2000).

Similar expression systems for scFv have been described for Saccharomyces cerevisiae, Schizosaccharomyces pombe, Yarrowia lipolytica, and Kluyveromyces lactis. Horwitz et al., Proc. Natl. Acad. Sci. USA 85:8678-8682 (1988); Davis et al., Biotechnology 9:165-169 (1991); and Swennen et al., Microbiology 148:41-50 (2002). Filamentous fungi, such as Trichoderma and Aspergillus, have the capacity to secrete large amounts of proteins. This property may be exploited for the expression of scFv and V_{HH}S. Radzio et al., Process-biochem. 32:529-539 (1997); Punt et al., Trends Biotechnol. 20:200-206 (2002); Verdoes et al., Appl. Microbiol. Biotechnol. 43:195-205 (1995); Gouka et al., Appl. Microbiol. Biotechnol. 47:1-11 (1997); Ward et al., Biotechnology 8:435-440 (1990); Archer et al., Antonie Van Leeuvenhoek 65:245-250 (1994); Durand et al., Enzyme Microb. Technol. 6:341-346 (1988); Keranen et al., Curr. Opin. Biotechnol. 6:534-537 (1995); Nevalainen et al., J. Biotechnol. 37:193-200 (1994); Nyyssonen et al., Biotechnology 11:591-595 (1993); and Nyyssonen et al., PCT WO 92/01797 (1992).

As discussed above, the present invention provides methods for using one or more of the inventive FGFR5 polypeptides or polynucleotides, FGFR5 agonists or antagonists, and modulators of FGFR5 expression to treat a disorder in a patient. As used herein, a “patient” refers to any warm-blooded animal, preferably a human.

In this aspect, the FGFR5 polypeptide or polynucleotide, modulator of FGFR5 gene expression or FGFR5 agonist or antagonist (referred to as the “active component”) is generally present within a composition, such as a pharmaceutical or immunogenic composition. Such compositions may comprise one or more active components and a physiologically acceptable carrier. Immunogenic compositions may comprise one or more of the active components and an immunostimulant, such as an adjuvant or a liposome, into which the active component is incorporated.

Alternatively, a composition of the present invention may contain DNA encoding one or more polypeptide active components described above, such that the polypeptide is generated in situ. In such compositions, the DNA may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus Calmette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

Routes and frequency of administration, as well as dosage, vary from individual to individual. In general, the inventive compositions may be administered by injection (e.g., intradermal, intramuscular, intravenous or subcutaneous), intranasally (e.g., by aspiration) or orally. In general, the amount of polypeptide present in a dose (or produced in situ by the DNA in a dose) ranges from about 1 pg to about 100 mg per kg of host, typically from about 10 pg to about 1 mg per kg of host, and preferably from about 100 pg to about 1 μg per kg of host. Suitable dose sizes will vary with the size of the patient, but will typically range from about 0.1 ml to about 2 ml.

While any suitable carrier known to those of ordinary skill in the art may be employed in the compositions of the present invention, the type of carrier will vary depending on the mode of administration. For parenteral administration, such as subcutaneous injection, the carrier preferably comprises water, saline, alcohol, a lipid, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the compositions of the present invention to non-specifically enhance the immune response. Most adjuvants contain a substance designed to protect the antigen from rapid catabolism, such as aluminum hydroxide or mineral oil, and a non-specific stimulator of immune responses, such as lipid A, Bordetella pertussis or M. tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A and Quil A.

The following examples are offered by way of illustration, not limitation.

### EXAMPLE 1

### Isolation of cDNA Sequences from Murine Lymph Node Stromal Cell Expression Libraries

The cDNA sequences of the present invention were obtained by high-throughput sequencing of cDNA expression libraries constructed from murine fsn -/- lymph node stromal cells as described below.

Lymph nodes were removed from flaky skin fsn -/- mice, the cells dissociated and the resulting single cell suspension placed in culture. After four passages, the cells were harvested. Total RNA, isolated using TRIzol Reagent (BRL Life Technologies, Gaithersburg, Md.), was used to obtain mRNA using a Poly(A) Quik mRNA isolation kit (Stratagene, La Jolla, Calif.), according to the manufacturer's specifications. A cDNA expression library (referred to as the MLSA library) was then prepared from the mRNA by Reverse Transcriptase synthesis using a Lambda ZAP Express cDNA library synthesis kit (Stratagene, La Jolla, Calif.). A second cDNA expression library, referred to as the MLSE library, was prepared exactly as above except that the cDNA was inserted into the mammalian expression vector pcDNA3 (Invitrogen, Carlsbad Calif.).

The nucleotide sequence of a cDNA clone isolated from the MLSA library is given in SEQ ID NO: 1, with the corresponding amino acid sequence being provided in SEQ ID NO: 5.

### EXAMPLE 2

### Characterization of Isolated cDNA Sequences

The isolated cDNA sequences were compared to sequences in the EMBL DNA database using the computer algorithm BLASTN, and the corresponding polypeptide sequences (DNA translated to protein in each of 6 reading frames) were compared to sequences in the SwissProt database using the computer algorithm BLASTP. Specifically, comparisons of DNA sequences provided in SEQ ID NO: 1 and 2-4 (isolated as described below) to sequences in the EMBL (Release 60, September 1999) DNA database, and the amino acid sequences correspoding to SEQ ID NO: 1-4 (provided in SEQ ID NO: 5-8, respectively) to sequences in the SwissProt and TrEMBL (up to Oct. 20, 1999) databases were made as of Dec. 31, 1999. The cDNA sequences of SEQ ID NO: 1-4, and their corresponding polypeptide sequences (SEQ ID NO: 5-8, respectively) were determined to have less than 75% identity (determined as described above) to sequences in the EMBL and SwissProt databases using the computer algorithms BLASTN and BLASTP, respectively.

Using automated search programs to screen against sequences coding for known molecules reported to be of therapeutic and/or diagnostic use, the isolated polynucleotides of SEQ ID NO: 1-4 were determined to encode polypeptide sequences that are members of the fibroblast growth factor (FGF) receptor family (SEQ ID NO: 5-8). A family member is herein defined to have at least 20% identical amino acid residues in the translated polypeptide to a known protein or member of a protein family.

Fibroblast growth factor receptors belong to a family of four single membrane-spanning tyrosine kinases (FGFR1 to 4). These receptors serve as high-affinity receptors for 23 growth factors (FGF1 to 23). FGF receptors have important roles in multiple biological processes, including mesoderm induction and patterning, cell growth and migration, organ formation and bone growth (Xu, Cell Tissue Res. 296:33-43, 1999). Further analysis of the sequence revealed the presence of a putative transmembrane domain and intracellular domain, similar to other FGF receptors.

### EXAMPLE 3

### Isolation of Full Length cDNA Sequence of a Murine Fibroblast Growth Factor Receptor Homolog

The full-length cDNA sequence of a murine fibroblast growth factor receptor homolog was isolated as follows.

The MLSA cell cDNA library (described in Example 1) was screened with an [α ³²P]-dCTP labeled cDNA probe corresponding to nucleotides 1 to 451 of the coding region within SEQ ID NO: 1. Plaque lifts, hybridization and screening were performed using standard molecular biology techniques. The determined polynucleotide sequence of the full-length murine FGFR gene (referred to as muFGFR5β) is provided in SEQ ID NO: 2, with the corresponding polypeptide sequence being provided in SEQ ID NO: 6.

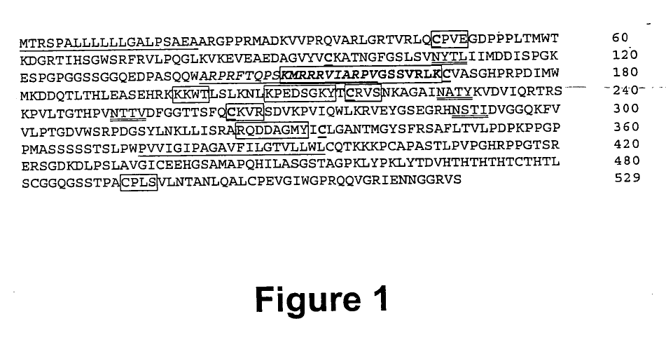

Analysis of the polynucleotide sequence of SEQ ID NO: 2 revealed the presence of a putative transmembrane domain encoded by nucleotides 1311 to 1370. The polypeptide sequence (SEQ ID NO: 6; FIG. 1) has regions similar to the extracellular domain of the fibroblast growth factor receptor family. The amino acid sequence of the extracellular domain of muFGFR5β is provided in SEQ ID NO: 13, while the amino acid sequence of the intracellular domain is provided in SEQ ID NO: 14.

A splice variant of SEQ ID NO: 2 was also isolated from the MLSA cDNA library as described in Example 1. The determined polynucleotide sequence of the splice variant (referred to as FGFR5γ) is provided in SEQ ID NO: 3 and the corresponding polypeptide sequence is provided in SEQ ID NO: 7. The splice regions are in an equivalent position to splice sites for previously described FGF receptors (Ornitz, J. Biol. Chem. 296:15292-15297 (1996); Wilkie, Current Biology 5:500-507 (1995); Miki, Proc. Natl. Acad. Sci. USA 89:246-250 (1992), thus establishing that this molecule (referred to herein as FGFR5) is a FGF receptor homolog. The main difference between the two FGFR5 splice variants is that muFGFR5β contains three extracellular Ig-domains, while FGFR5γ contains only two such domains.

To examine the structural similarities between FGFR5γ and FGFR5β and the other members of the FGF receptor family, 3D Swiss modeller (Petisch, Bio/Technology 13:658-660 (1995); Peitsch, Biochem Soc Trans. 24:274-279 (1996); and Guex and Peitsch, Electrophoresis 18:2714-2723 (1997)) was employed to produce a predicted crystal structure of the extracellular domain of FGFR5γ. These studies showed that the crystal structure of FGFR5 deviates from that of the known FGFR1 structure between residues 188 and 219 of SEQ ID NO: 7 (provided in SEQ ID NO: 15). These residues correlate with an area of low homology between FGFR5 and other members of the FGF receptor family that may have a critical role in defining ligand specificity.

The critical residues for ligand binding have previously been identified in co-crystallization studies of FGFR1 binding FGF-2 (Plotnikov et al., Cell 98:641-650 (1999)). Alignment of FGFR5γ with FGFR1 showed that many of these residues are conserved or are a conservative substitution. Conserved ligand binding residues between the two receptors are found at residues 66, 68, 146, 178, 181, 183 and 216 of SEQ ID NO: 7, while conservative substitutions of potential ligand binding residues are found at residues 64, 180 and 226 of SEQ ID NO: 7. When visualized on the predicted crystal structure of FGFR5γ, these residues line the groove of the ligand binding domain. Thus, while the overall degree of similarity between FGFR5 and other FGF receptors (i.e. FGFR 1-4) is relatively low, the extracellular domains of the FGFR5 splice variants have all the conserved residues important for ligand binding.

The main difference between the FGFR5 receptor and other family members is the lack of an intracellular tyrosine kinase domain. With the four previously identified FGF receptors (FGFR1-4), signal transduction is mediated by ligand binding and receptor dimerization, resulting in autophosphorylation of the tyrosine residues within the intracellular RTK domain and phosphorylation of a number of intracellular substrates, initiating several signal transduction cascades. The FGFR5β and FGFR5γ splice variants described herein both contain tyrosine residues in the intracellular domain demonstrating similarity to a SHP binding motif (residues 458-463 of SEQ ID NO: 6 and 367-377 of SEQ ID NO: 7). SHPs are protein tyrosine phosphatases that participate in cellular signalling and that have previously been identified in the cytoplasmic domains of many receptors eliciting a broad range of activities. The presence of such motifs in the cytoplasmic domain of FGFR5 is thus indicative of signaling, and modification of these motifs may be employed to modulate signal transduction initiated by binding of a ligand to FGFR5. These motifs are conserved between the mouse FGFR5s and the human homologs described below (Example 4). Removal or modification of these signaling motifs and/or the cytoplasmic domain of FGFR5 may be employed to engineer a soluble FGFR5-like molecule that binds to the FGFR5 ligand without stimulating signaling. Such a molecule may be usefully employed to modulate the binding, and therefore activity, of FGFR5.

### EXAMPLE 4

### Isolation of a Human FGF Receptor Homolog

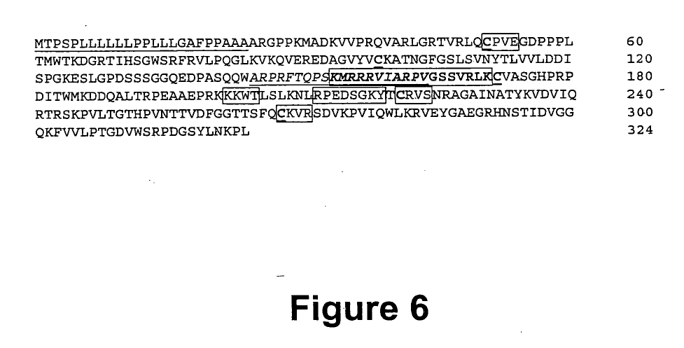

The cDNA encoding the partial murine FGF receptor (SEQ ID NO: 1) was used to search the EMBL database (Release 58, March 1999) to identify human EST homologs. The identified EST (Accession Number A1245701) was obtained from Research Genetics, Inc (Huntsville Ala.) as I.M.A.G.E. Consortium clone ID 1870593. Sequence determination of the complete insert of clone 1870593 resulted in the identification of 520 additional nucleotides. The insert of this clone did not represent the full-length gene. The determined nucleotide sequence of the complete insert of clone 1870593, which represents the extracellular domain of the human FGF receptor homolog, is given in SEQ ID NO: 4 and the corresponding polypeptide sequence is provided in SEQ ID NO: 8. Several conserved domains were identified in SEQ ID NO: 8 that are involved in the dimerization, ligand binding and activity of the receptor. These are shown in FIG. 6. The full-length amino acid sequence for human FGFR5 is provided in SEQ ID NO: 153, with the corresponding cDNA sequence being provided in SEQ ID NO: 154.

Both murine and human FGFR5 are structurally similar to FGFR1-4, the other members of the FGFR family. In the extracellular domain, three immunoglobulin-like motifs are present that are flanked by conserved cysteine residues. The Ig-1 loop is the least conserved of the three Ig loops and is not required for ligand binding, but regulates binding affinity (Shi et al., Mol. Cell. Biol. 13:3907-3918 (1993)). The Ig-3 loop is involved in ligand selectivity (Ornitz et al., Science 268:432-436 (1996)).

An acidic box is characteristic in FGFR1-4 and is involved in binding divalent cations, including copper and calcium. Acidic boxes are important for interaction with cell adhesion molecules, extracellular matrix and heparin (Patstone and Maher, J. Biol. Chem. 271:3343-3346 (1996)). The acidic box in FGFR5 is smaller than in the other four receptors or absent.

The cell adhesion molecule (CAM) homology and heparin-binding domain is also characteristic of the extracellular domain (Szebenyi and Fallon, Int. Rev. Cytol. 185:45-106 (1999)). The CAM homology region is a binding site for L1, N-CAM and N-cadherin (Doherty et al., Perspect Dev Neurobiol. 4(2-3):157-68 (1996)).

The FGFR5 heparin-binding domain is typical of other FGFR heparin-binding domains and consists of a cluster of basic and hydrophobic residues flanked by Lys residues (Kan et al., Science 259:1918-1921 (1993)). Heparin or heparan sulfate proteoglycans are essential co-factors for the interaction of FGFs with FGFRs and it has been shown that heparin is a growth-factor independent ligand for FGFR4 (Gao and Goldfarb, EMBO J. 14:2183-2190 (1995)).

### EXAMPLE 5

### Characterization of the Murine FGF Receptor Homolog

Soluble forms of the murine FGF receptor homolog muFGFR5β and splice variant FGFR5γ (SEQ ID NO: 2 and 3, respectively) were expressed in mammalian cells and the purified proteins used to determine the ligand binding specificity of the receptor molecules as follows.

The extracellular domains of muFGFR5β and FGFR5γ were amplified by PCR using primers MS158 and MS159 (SEQ ID NO: 10 and 11, respectively) and cloned into the expression vector pcDNA3 containing the Fc fragment from human IgG1. These soluble recombinant proteins, referred to as FGFR5βFc and FGFR5γFc, were expressed in HEK293 cells (ATCC No. CRL-1573, American Type Culture Collection, Manassas, Va.) and purified using an Affiprep protein A column (Biorad, Hercules Calif.).

FGF-2 (basic fibroblast growth factor) has previously been demonstrated to bind all FGF receptors but with a range of affinities. Binding of muFGFR5β to FGF-2 was demonstrated by co-incubating the purified protein and FGF-2 in the presence of protein G Sepharose (Amersham Pharmacia, Uppsala, Sweden) and resolving complexes formed on denaturing polyacrylamide gels. FGF-2 (2 μg) was incubated with 5 μg FGFR5βFc, FGF Receptor 2 (FGFR2Fc) or unrelated protein (MLSA8790Fc) in 5 μl protein G Fast Flow beads (Pharmacia, Uppsala, Sweden), PBS and 0.1% Triton X-100 for 60 min at 4° C. The beads were washed three times in 0.1% Triton X-100/PBS and resuspended in 20 μl loading buffer (0.1 M DTT, 10% sucrose, 60 mM Tris.HCl pH 6.8, 5% SDS and 0.01% bromophenol blue). The samples were analysed on a 12% polyacrylamide gel. FGF-2, FGFR2Fc, FGFR5βFc and MLSA8790Fc (1 μg of each) were loaded on the gel for comparison. After staining of the gel with Coomassie blue, a doublet of bands were visible in the lane containing FGFR5βFc, indicating that a complex formed between the FGF-2 and the murine FGF receptor homolog FGFR5βFc, and that FGF-2 is a ligand for the novel FGF receptor homolog. A doublet was also observed in the lane containing the FGFR2Fc, which was the positive control. No doublet was observed in the negative control lane containing the MLSA8790Fc protein.

The binding specificity of the murine FGF receptor homolog FGFR5βFc was further examined by repeating the experiment described above, replacing the FGF-2 with another known growth factor, epidermal growth factor (EGF). In this experiment, EGF did not bind to FGFR2Fc, FGFR5βFc or MLSA8790Fc, indicating that binding of FGF-2 to the murine FGF receptor homolog FGFR5βFc was specific. Similarly, in subsequent experiments employing FGF-7, no binding of FGFR2Fc, FGFR5βFc or MLSA8790Fc was observed.

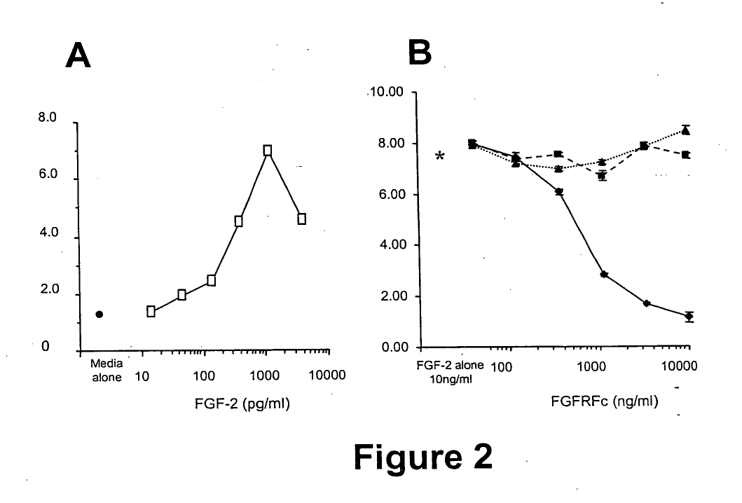

To determine the difference in binding affinity between FGFR5 and FGFR2, the ability of FGFR5βFc and FGFR5γFc to inhibit FGF signalling in FGF-responsive NIH-3T3 SRE reporter cells was examined. Fibroblast growth factors typically signal via phosphorylation of the receptor tyrosine kinase domain stimulating the MAP kinase pathway. This eventually leads to activation of genes under the control of the serum response element (SRE). Reporter constructs containing concatamerized SRE sequences upstream of a luciferase reporter gene were stably transfected into NIH-3T3 cells. Reporter activity was measured by measuring luciferase levels. As shown in FIG. 2A, a dose dependent response of NIH-3T3 SRE cells to FGF-2 was seen in the presence of heparin. Using a standard dose of FGF-2 in the presence of heparin, an increasing concentration of FGFR2Fc, FGFR5βFc or FGFR5γFc was titrated onto the NIH-3T3 SRE cells and luciferase activity was measured. Increasing concentrations of FGFR2Fc, the positive control, reduced the luciferase signal in FGF-2 stimulated cells (FIG. 2B). However, titrating FGFR5βFc and FGFR5γFc did not inhibit FGF-mediated luciferase signal from the NIH-3T3 SRE cells. These results show that FGF-2 has lower affinity for either FGFR5β or FGFR5γ than for FGFR2, and indicate that the ligand specificity of FGFR5 is different to those of the other members of the FGF receptor family.

### EXAMPLE 6

### Sequence Determination of a Polynucleotide Fragment Containing Genomic Murine FGFR5β

As noted above, the two splice variants muFGFR5β and muFGFR5γ do not contain the classical receptor tyrosine kinase domain present in other known FGF receptors. In order to investigate the existence of a splice variant of FGFR5 containing a classical receptor tyrosine kinase (RTK) domain, the genomic DNA of FGFR5 was cloned and sequenced as follows.

Mouse genomic DNA was isolated from L929 cells using standard techniques. A genomic polynucleotide fragment containing murine FGFR5β was PCR amplified using primers MS157 and MS166 (SEQ ID NO: 11 and 12, respectively). The 1.4 kb polynucleotide fragment was cloned into a T-tailed pBluescript SK²⁺ vector. The sequence of the insert of this plasmid was determined using standard primer walking sequencing techniques. The sequence of the genomic fragment containing murine FGFR5β is given in SEQ ID NO: 9. This sequence extends from the 3′ untranslated region to the sequence encoding the 5′ end of the mature FGFR5 receptor minus the signal sequence. No alternative exons expressing an RTK domain were identified.

### EXAMPLE 7

### Stimulation of Cell Growth by Murine FGFR5β and FGFR5γ

RAW264.10 cells are derived from a murine macrophage cell line generated from BALB/c mice, and are macrophage and osteoclast precursors. Stimulation of RAW264.10 cells (Hamilton et al., J. Exp. Med. 148:811-816 (1978)) and peripheral blood mononuclear cells (PBMC) in the presence of the murine FGFR5β and FGFR5γ (also referred to herein as FGFRβ and FGFRγ, respectively) was demonstrated as follows.

The murine FGF receptor homolog, muFGFR5β, and splice variant FGFR5γ (SEQ ID NO: 2 and 3, respectively) were expressed in mammalian cells and purified as murine FGFR5β-Fc and FGFR5γ-Fc fusion proteins as described above. The FGFR5β- and FGFR5γ-Fc fusion proteins were titrated from 10 nM in 0.05 ml media (DMEM supplemented with 5% FBS, 2 mM L-glutamine (Sigma, St Louis Mo.), 1 mM sodium pyruvate (Life Technologies, Gibco BRL, Gaithersburg Md.), 0.77 mM L-asparagine (Sigma), 0.2 mM arginine (Sigma), 160 mM penicillin G (Sigma), 70 mM dihydrostreptomycin sulfate (Boehringer Mannheim, Roche Molecular Biochemicals, Basel, Switzerland) in a 96-well flat-bottomed microtitre plate. Purified human FGFR2-Fc fusion protein was used as control and titrated from 10 nM.

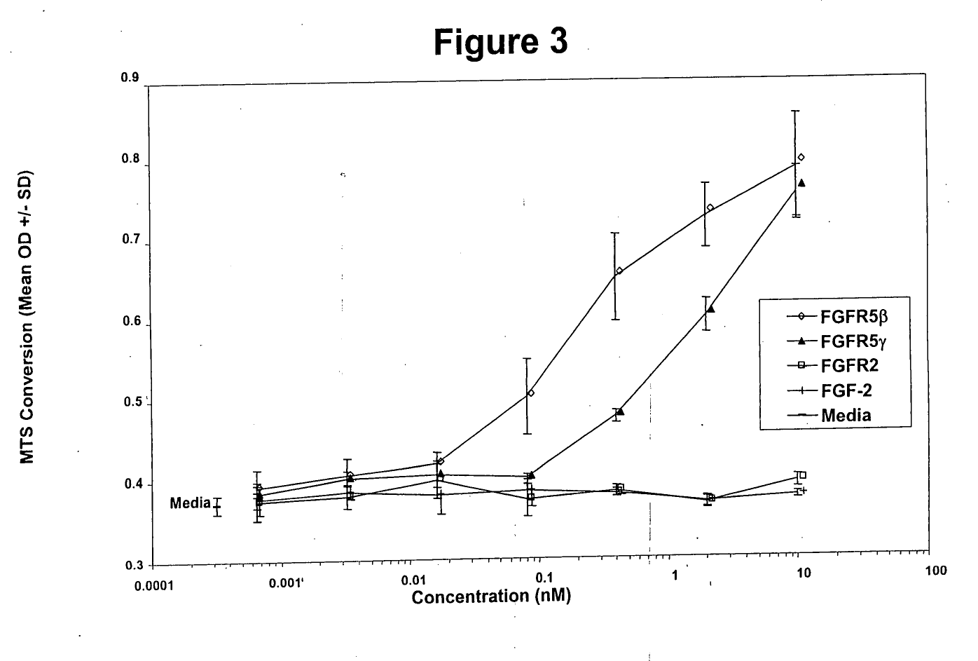

RAW264.10 cells were added to each well in 0.05 ml media at a concentration of 2×10⁴ cells/ml. The plate was incubated at 37° C. in a humidified atmosphere containing 10% CO₂ for 4 days. Cell growth was determined by MTS dye conversion and quantified using an ELISA reader. As shown in FIG. 3, both murine FGFR5β-Fc and FGFR5γ-Fc fusion proteins stimulated the growth of RAW264.10 cells at concentrations of 100 pM and greater of Fc fusion protein.

These results demonstrated that FGFR5β and FGFR5γ are immunostimulatory molecules that directly activate a macrophage cell line. The macrophage cell line used in these assays (RAW264.10) has previously been shown to differentiate into osteoclasts when stimulated with a variety of known bone morphogenic agents. The effects of FGFR5β and FGFR5γ on these cells indicate that these molecules may also stimulate the differentiation and activation of osteoclasts, which are associated with the resorption and remodelling of bone. Weidemann and Trueb (Genomics 69:275-279 (2000)), have shown that FGFR5 is expressed in cartilaginous tissues. When viewed in the context of the data provided above, this indicates that FGFR5 may play a role in bone formation and may therefore have applications in fracture repair and bone diseases, such as osteoporosis and osteopetrosis.

### EXAMPLE 8

### Stimulation of Proliferation and Adherent Peripheral Blood Mononuclear Cells (PBMC) by Murine FGFR5β and FGFR5γ

Stimulation of PBMC to adhere to plastic by murine FGFR5β, murine FGFR5γ Fc and human FGFR5β-Fc fusion proteins was demonstrated as follows.

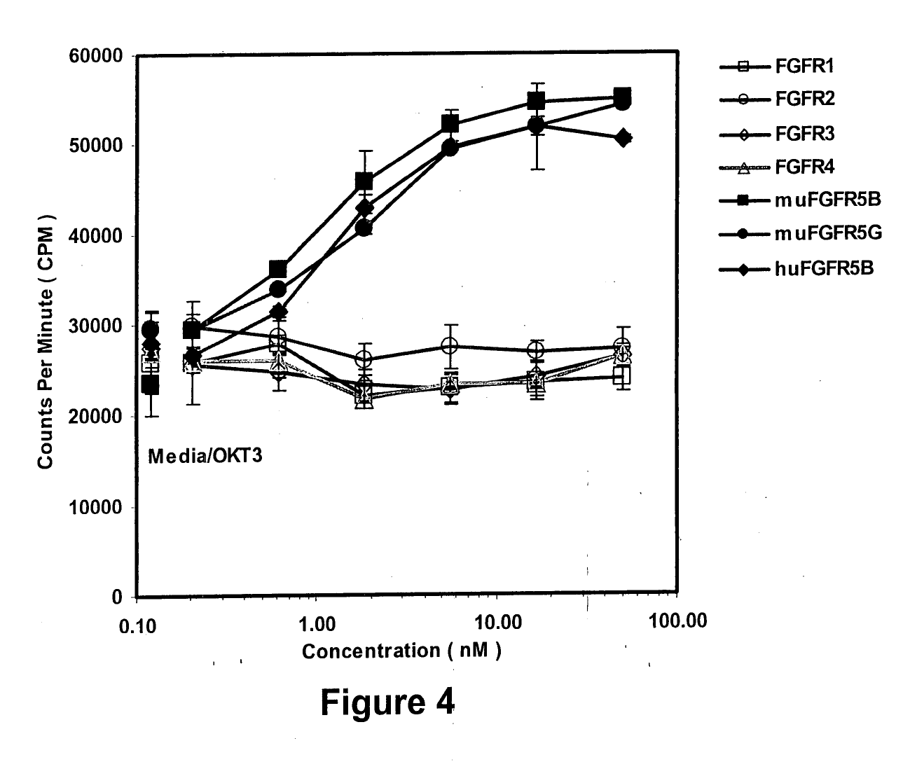

Purified murine FGFR5β-Fc, murineFGFR5γ-Fc and human FGFR5β-Fc fusion proteins were titrated from 100 nM into 0.1 ml media per well of 96 well microtiter plates. Purified human FGFR1, 2, 3, and 4-Fc fusion proteins were used as controls. PBMC were harvested from blood by density gradient centrifugation and resuspended in media to a concentration of 2×10⁶ cells/ml. Antibodies to CD3 (OKT3) or media were added to the PBMC and 0.1 ml of cells dispensed to each well. The plates were incubated for 3 days at 37° C. in a humidified atmosphere containing 5% CO₂ in air. Cell proliferation was quantified by pulsing the plates with tritiated (³H)-thymidine for the final 16 hours of culture. The cells were then harvested and ³H-thymidine incorporation quantified by standard liquid scintillation counting. FIG. 4 shows that murine and human FGFR5β, and murine FGFR5γ fusion proteins enhanced proliferation of PBMCs activated with anti-CD3 but did not induce the proliferation of PBMC on their own (data not shown). Proliferation was not stimulated with human FGFR1, 2, 3, or 4-Fc fusion proteins.

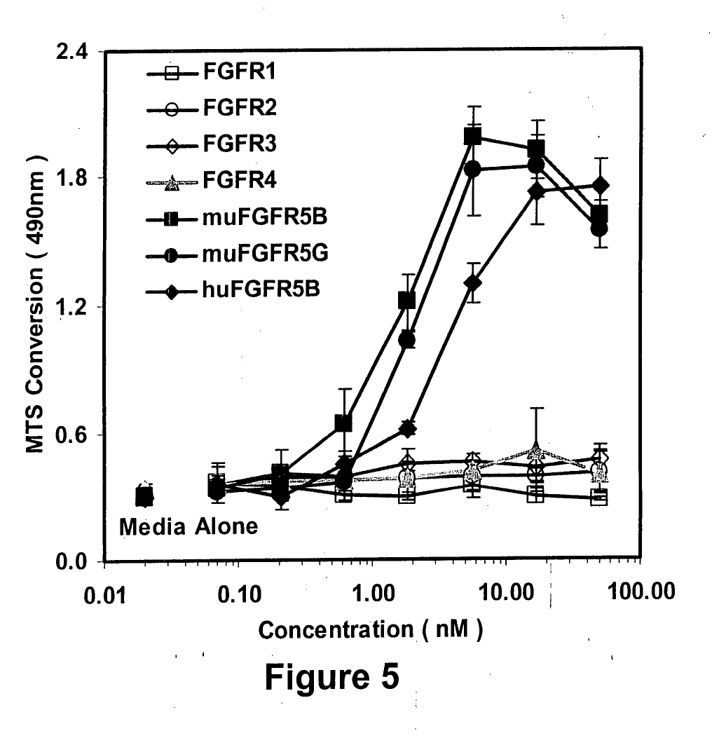

MuFGFR5β, muFGFR5γ and human FGFR5β (SEQ ID NO: 2, 3 and 4, respectively) were expressed in mammalian cells and purified as Fc fusion proteins as described above. The muFGFR5β-Fc, muFGFR5γ-Fc and human FGFR5β-Fc fusion proteins were titrated from 100 nM into 0.1 ml media per well of 96 well microtitre plates. Peripheral blood mononuclear cells (PBMC) were harvested from blood by density gradient centrifugation and resuspended in media to a concentration of 2×10⁶ cells/ml. PHA or media (RPMI 1640 supplemented with 5% FBS, 2 mM L-glutamine (Sigma), 160 mM penicillin G (Sigma), and 70 mM dihydrostreptomycin sulfate (Boehringer Mannheim) was added to the PBMC and 0.1 ml of cells dispensed to each well. The plates were incubated for 3 days at 37° C. in a humidified atmosphere containing 5% CO₂ in air. The non-adherent cells were removed with three media washes. Media (0.05 ml) containing MTS/PES solution (CellTiter96 Aqueous One Solution Cell Proliferation Assay, Promega, Madison, Wis.) was dispensed to each well and the plate incubated for 4 hrs before the degree of dye conversion was quantified using a 96-well ELISA plate reader. FIG. 5 shows that muFGFR5β, muFGFR5γ Fc and human FGFR5β-Fc fusion proteins stimulated, in a dose dependent manner, the adherence of PBMC as well as the proliferation of the adherent PBMC. These results demonstrate that FGFR5β and FGFR5γ are capable of enhancing the proliferative effects of known immunostimulatory molecules on a mixed population of human haemopoietic cells, namely PBMC.

### EXAMPLE 9

### FGFR5 Activates Human Monocyte-Derived Macrophage with a Unique Phenotype

This Example discloses the activation of human monocyte derived macrophage by murine FGFR5β-Fc. The stimulation of peripheral blood mononuclear cells with FGFR5β-Fc leads to the growth of a population of adherent cells. The phenotype of these cells was determined by staining with a panel of monoclonal antibodies to lineage-specific and activation markers. PBMC were cultured with FGFR5β-Fc, FGFR2-Fc or media for 3 days and the adherent cells harvested by treatment with the Accutase (Sigma) enzyme solution. More than 90% of the cells were viable, as assessed by Trypan blue dye exclusion. These were stained with monoclonal antibodies specific for CD3, CD14, CD19 and CD33. All cells expressed the CD33 antigen, indicating that they were of the macrophage lineage. In contrast, very few cells could be harvested from the cultures incubated with FGFR2-Fc or media, although the majority of the cells collected from these cultures also expressed CD33.

Macrophages are highly plastic cells that can assume a number of functionally different phenotypes. The phenotype of the macrophage is dictated by the factor used to activate the cell. Thus the IL-4 activated macrophage is phenotypically distinct from the macrophage activated by either IFNγ or LPS. FGFR5β-Fc was compared with other known macrophage stimulants to determine whether it could be characterized as an IFNγ or IL-4-like macrophage stimulant. Monocyte-derived macrophages (MDM) were collected from PBMC by adherence to plastic, and stimulated with FGFR5β-Fc, FGFR2-Fc, IL-4, IFNγ or LPS for 48 hrs. Following collection from the culture dishes, they were washed and stained with antibodies to the following cell surface markers: CD1a, CD3, CD14, CD16, CD23, CD32, CD33, CD40, CD56, CD80, CD83, CD86, CD206 and HLA-DR. The FGFR5β-Fc-activated MDM expressed a unique profile of cell surface antigens that did not match that of other stimulants. Most strikingly, FGFR5β-Fc stimulated the up-regulation of the cell adhesion molecule CD56. This has been observed on at least four occasions and confirmed by quantitative RT-PCR analysis of mRNA expression. CD56 expression is normally associated with neural cells, NK cells, or myeloid or B cell leukemia but not macrophage. The significance of this observation is not clear but it would be of interest to determine whether macrophage from SLE patients or other immune-mediated diseases express CD56.

### EXAMPLE 10

### Stimulation of Gene Expression in Human Monocytes by Murine FGFR5β-Fc Fusion Protein

This Example discloses genes that were overexpressed in human monocytes stimulated with the murine FGFR5β-Fc fusion protein.

Monocytes were purified from human peripheral blood mononuclear cells (PBMC) by adherence for 2 hours at 37° C. Cells were stimulated with 100 nM of soluble FGFR5β human IgG Fc fusion protein or soluble FGFR2 human IgG Fc fusion protein. After 0 and 12 hours the adherent monocytes were collected and total RNA extracted from the cells using Trizol reagent (Invitrogen Corp., Carlsbad Calif.) following the manufacturer's instructions. The RNA was amplified and aminoallyl UTP incorporated using the Ambion MessageAmp aRNA kit (Ambion Inc, Austin Tex.) following the manufacturer's instructions.

The extracted amplified RNA from the FGFR5β and FGFR2-treated cells was labelled with either Cy3 or Cy5 dye (Amersham Pharmacia Biotech, Buckinghamshire UK), respectively, by indirect aminoallyl dUTP labeling and hybridized to 2 Clontech Atlas Glass 3.8 gene microarrays (BD Biosciences Clontech, Palo Alto, Calif.). The slides were washed, scanned and analyzed using Axon GenePix scanner and software (Axon Instruments Inc., Union City, Calif.). Where indicated, quantitative PCR was used to validate the microarray data and quantify the mRNA for genes not present on the array. Primers and probe sets were purchased from Perkin Elmer/Applied Biosystems (Foster City, Calif.) and MWB Biotech (Ebersberg, Germany) and all PCR reactions were run on a Perkin Elmer/Applied Biosystems 7700 following the manufacturer's instructions.

Treatment of monocytes with FGFR5β-Fc up-regulated expression of the 26 genes listed in Table 1 below. The up-regulation of three of the genes was confirmed by quantitative PCR. In addition, the expression of eight human cytokines was analyzed by quantitative PCR and the results of this analysis are shown in Table 1.

FGFR5-Fc stimulated a dramatic up-regulation in the levels of osteopontin (OPN) and TGFβ but had only modest effects on the other cytokines. This profile of gene expression was very unlike that described for other stimulators of monocytes such as LPS, Mycobacterium tuberculosis, GM-CSF and M-CSF, which stimulate modest OPN expression but pronounced expression of pro-inflammatory cytokines such as IL-1β, IL-6, IL-8 IL-10, IL-12 and TNFα (Rosenberger et al., J. Immunol. 164:5894-904 (2000); Suzuki et al., Blood 96:2584-2591 (2000); Hashimoto et al., Blood 94:837-844 (1999); Hashimoto et al., Blood 94:845-852 (1999); Boldrick et al., Proc. Natl. Acad. Sci. USA 99:972-977 (2002); Ragno et al., Immunol. 104:99-108 (2001)).

TABLE 1
Genes up-regulated in monocytes following treatment with FGFR5

| | | Microarray | Quantitative PCR |
|---|---|---|---|
| Secreted Molecules | GENBANK | Fold up-regulation | Fold up-regulation |
| Osteopontin | NM_000582 | 4.95 | 48.4 |
| Interferon, alpha 8 | NM_002170 | 2.27 | ND |
| EXODUS | NM_004591 | 2.27 | 6.3 |
| IL-1β | XO2532 | Not Determined (ND) | 3.4 |
| IL8 | NM_000584 | ND | 5.5 |
| IL-10 | NM_000572 | ND | undetectable |
| IL-12 p35 | NM_000882 | ND | undetectable |
| IL-12p40 | NM_002187 | ND | undetectable |
| IL-20 | NM_018724 | ND | undetectable |
| TGFβ | NM_000660 | ND | 27.3 |
| TNFα | XO1394 | ND | 4.0 |
| Channels and Receptors | | | |
| MICA | NM_000247 | 2.08 | 4.7 |
| TIE1 | NM_005424 | 3.30 | ND |
| Calcium channel, voltage- | NM_000726 | 2.44 | ND |
| dependent, beta 4 subunit | | | |
| LDL receptor-related protein 8 | NM_004631 | 2.20 | ND |
| Cytoskeletal Molecules | | | |
| Myosin VI | NM_004999 | 1.89 | ND |
| Myosin, heavy polypeptide 1 | NM_005963 | 2.12 | ND |
| Troponin C, slow | NM_003280 | 1.88 | ND |
| Kinectin 1 kinesin receptor | NM_004986 | 1.73 | ND |
| Signalling Molecules | | | |
| Protein kinase C, iota | NM_002740 | 2.26 | ND |
| Protein tyrosine phosphatase, | NM_002833 | 1.85 | ND |
| non-receptor type 9 MEG-2 | | | |
| Importin alpha 6 | NM_002269 | 2.17 | ND |
| Protein kinase, X-linked | NM_005044 | 1.92 | ND |
| Suppression of tumorigenicity 5 | NM_005418 | 3.16 | ND |
| RAR-related orphan receptor B | NM_006914 | 2.08 | ND |
| Zinc finger protein 124 HZF-16 | NM_003431 | 2.94 | ND |
| Metabolism | | | |
| Ubiquitin-conjugating enzyme | NM_003341 | 2.41 | ND |
| Transplantation antigen P35B | NM_003313 | 2.48 | ND |
| UDP glycosyltransferase 2 | NM_001075 | 2.35 | ND |
| Alcohol dehydrogenase 2 | NM_000668 | 2.41 | ND |
| Solute carrier family 18 vesicular | NM_003053 | 2.07 | ND |
| monoamine, member 1 | | | |
| Seryl-tRNA synthetase | NM_006513 | 1.88 | ND |
| Other | | | |
| H1 histone family, member 1 | NM_005325 | 1.99 | ND |
| Chr. 8 open reading frame 1 | NM_004337 | 2.08 | ND |

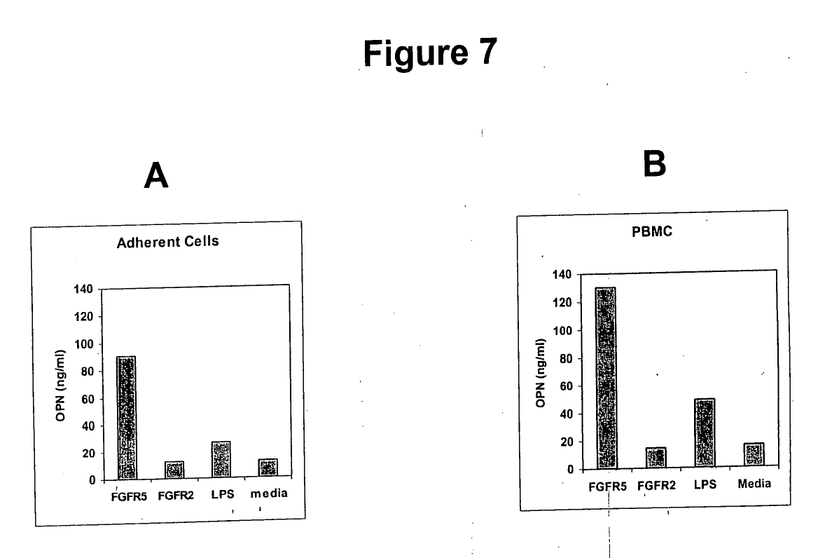

In addition to demonstrable upregulation of OPN mRNA, PBMC and adherent PBMC (predominantly monocytes) were stimulated with FGFR2, FGFR5, LPS or media alone for 24 hours and the supernatants collected for cytokine analysis. LPS induced the production of the expected pro-inflammatory cytokines such as IL-1, IL-6 and TNFα whereas FGFR5 did not. In contrast, FGFR5 stimulated both PBMC and adherent PBMC to produce 90 and 130 ng/ml of osteopontin, respectively. LPS stimulated 20 and 50 ng/ml of osteopontin, and FGFR2 and the media control cultures contained less than 20 ng/ml of OPN. See, FIG. 7A-B. These results are consistent with the microarray and real time PCR results presented in Table 1, above, and demonstrate that FGFR5 selectively stimulated osteopontin production by PBMC.

A second microarray analysis of genes up-regulated by FGFR5 was performed using the Affymetrix, Inc. (Santa Clara, Calif.) Gene Chip microarray technology. Adherent human PBMC were stimulated with media, FGFR2-Fc or FGFR5-Fc for 12 hours and the RNA was collected, amplified, and labelled with a fluorescent dye. The labelled RNA was hybridized to Gene Chips printed with oligonucleotides that represent all of the genes in the human transcriptome. Fluorescently labelled cRNA were generated using the protocols provided by Affymetrix and the labelled RNA was hybridized to the chips.

150 genes up-regulated in monocyte-derived macrophages (MDMs) stimulated with FGFR5-Fc were identified that were not up-regulated in MDM treated with media alone or with FGFR2-Fc. An analysis of the genes up-regulated in MDM by FGFR5 reveals a pattern of gene expression which is similar to that described for IL-4 and IL-13 activated macrophage (see Table 2). The M2 macrophages, like those stimulated by FGFR5, do not express pro-inflammatory cytokines but express inhibitors of inflammation such as IL-1 receptor antagonist and the Decoy IL-1 receptor. These cells are known as alternatively activated, or M2, macrophage and are thought to have different functions to LPS or IFNγ activated macrophage (M1 macrophage). M2 macrophages are found in tumours and in allergic individuals, and are thought to play a role in tissue repair, whereas the M1 macrophages are the classically activated macrophage that engulf and kill bacteria (reviewed in Nature Reviews in Immunology 3:23-35 (2003)). The selective stimulation of M2 macrophage by FGFR5 administration may be beneficial in some therapeutic settings such as wound healing.

This microarray experiment also confirmed our previous observations that osteopontin and TGFβ1 were overexpressed and that CD14 was down-regulated following FGFR5 stimulation of MDM cells, and that many adhesion-associated genes were up-regulated. This observation is consistent with the growth and adhesion-promoting activity of FGFR5 on monocyte-derived macrophage (MDM) cells.

The microarray experiments identified the overexpression of the TNF superfamily member, LIGHT (aka TNFSF14), a known growth factor for activated T-cells that acts as a co-stimulant for these cells. Quantitative PCR was employed to confirm that LIGHT expression was upregulated in FGFR5-stimulated MDM cells. Without wishing to be limited to a specific mode of action, it is believed that the FGFR5-dependent over-expression of LIGHT in MDM cells may explain how FGFR5 augments the proliferation of anti-CD3 driven T-cell proliferation.

TABLE 2
Genes differentially expressed in M1 or M2 macrophage

| M1 Macrophage | M2 Macrophage |
|---|---|
| TLR2 and 4 | Scavenger receptor A and B |
| TNFα, IL-1, IL-6, IL-12 | CD163 |
| IL-1R Type I | Mannose Receptor |
| CXCL8, CXCL9, CXCL10, CXCL11 | CD23 |
| CCL2, CCL3, CCL4, CCL5 | IL-1 receptor antagonist |
| | Decoy IL-1 R type II |
| | CCL17, CCL22, CCL24 |
| | (Eotaxin 2) Arginase |

(Genes indicated by italics are upregulated in FGFR5 stimulated MDM)

In total, the results presented herein demonstrate that FGFR5 is a potent stimulator of osteopontin expression. Osteopontin (OPN) is a multifunction protein secreted by activated macrophages that shares most of the functions described herein for FGFR5. More specifically, OPN is a potent immunostimulatory molecule (O'Regan et al., Immunol. Today 21:475-478 (2000)) that stimulates macrophage adherence, activation, cytokine secretion and growth. It has been shown that OPN is a regulator of T-cell responses in that it augments CD3-induced proliferation, IFNγ production, and CD40 ligand expression. OPN also enhances Th1 and inhibits Th2 cytokine expression. It directly induces macrophages to produce IL-12 and inhibits IL-10 expression by LPS stimulated macrophages (Ashkar et al., Science 287:860-864 (2000)). OPN has also been shown to induce B cell proliferation and auto-reactive antibody production, and it appears that OPN may preferentially activate a CD5+ subset of B-cells and induce the production of auto-antibodies.

Osteopontin has been linked with a number of pathophysiological states including a variety of tumors; autoimmune diseases such as multiple sclerosis (MS), systemic lupus erythematosus (SLE), diabetes and rheumatoid arthritis; granulomatous inflammation such as sarcoidosis and tuberculosis; and pathological calcifications such as kidney stones and atherosclerosis (Giachelli and Steitz, Matrix Biol. 19:615-622 (2000)). Elevated levels of OPN are found in the sera of SLE patients and the autoimmune-prone MRL mice. Recently two groups described a central role for OPN in multiple sclerosis (Chabas et al., Science 294:1731-1735 (2001) and Jansson, J. Immunol. 168:2096-2099 (2002)). OPN is prevalent in the plaques of MS patients and, due to its immunostimulatory properties, it has been proposed that OPN plays a role in the progression of MS. This effect was demonstrated in experimental allergic encephalopathy (EAE), the murine model for MS. Mice that lacked the OPN gene were resistant to progressive EAE and had frequent remissions when compared to wild-type mice expressing OPN.

The chromosomal location of FGFR5 is 4p16. Genetic screens on large numbers of SLE patients show that a mutation at this location is associated with disease. FGFR5 sequence analysis may thus be used to identify individuals at risk of developing SLE by determining whether a mutation exists.

OPN has also been shown to function in bone remodelling by inhibiting calcification. Inhibition of OPN expression, by reducing the level or binding of FGFR5, may thus be useful in the treatment of osteoporosis.

Many of the effects described for FGFR5 may be mediated by its ability to induce high levels of osteopontin expression. Osteopontin is clearly a key molecule in the progression of a number of disease processes and therefore regulators of osteopontin expression, such as FGFR5, are targets for therapeutics for osteopontin-mediated diseases, including SLE, vasculitis, atherosclerosis, nephritis and arthritis.

### EXAMPLE 11

### Analysis of FGFR5 Expression Using FGFR5-Specific Polyclonal Antibodies

This example discloses the preparation of a rabbit anti-FGFR5 polyclonal antisera and its use in detecting the expression of FGFR5 protein in a variety of normal and disease tissues from humans.

Polyclonal antibodies were generated to the extracellular domain of FGFR5β by immunizing rabbits with murine FGFR5β extracellular domain fused to human IgG1 Fc fragment emulsified in complete Freund's adjuvant. The FGFR5-specific immune response was boosted by three subcutaneous injections at weekly intervals with the same protein and then twice with pure murine FGFR5β extracellular domain protein. Antisera were collected from the rabbits and the IgG purified by Protein A affinity chromatography.

Antibodies raised to the human IgG Fc portion of the immunogen were removed by absorption to Sephadex beads coated with human IgG. The resultant polyclonal antibody specifically reacted with human and mouse FGFR5 but did not recognize human FGFR1, 2, 3, or 4 Fc fusion proteins (purchased from R&D Systems, Minneapolis Minn.) in ELISA or by Western blotting.

Immunohistochemical analysis of human normal and diseased tissue arrays (SuperBioChips Laboratories, Seoul, Korea) revealed that FGFR5 was expressed in a minor population of granulocytes in the red pulp region of the spleen. FGFR5-expressing granulocytes were also found in a number of tissues, including the stomach, lung and small intestine. FGFR5 expression was also detected in skeletal muscle, skin and kidney. In addition, expression of FGFR5 was found in tissue biopsies from a hepatocellular carcinoma and a squamous cell carcinoma.

Diabetes

FGFR5 was detected in cells within the islets of Langerhans of the pancreas and may therefore play a role in diabetes (see, Kim et al. Biochim. Biophys. Acta 1518:152-156 (2001)), especially given the immunostimulatory properties of this molecule.

Rheumatoid Arthritis

Patients with rheumatoid arthritis often form inflammatory, granulomatous lesions under the skin that are referred to as rheumatoid nodules. Sections from rheumatoid nodules were stained and confirmed to express FGFR5.

Sarcoidosis

Sarcoidosis is thought to be an autoimmune disease that is characterized by the formation of non-caseating sterile granulomas. Granulomas are nodular lesions that form due to chronic localized stimulation of macrophages that differentiate into large epithelioid cells, histiocytes, and giant cells.

Two human sarcoidosis patient biopsy samples were cut and stained for FGFR5 expression. The first biopsy sample was a lymph node that was filled with numerous small granulomas surrounded by lymphoid tissue. The granulomas expressed FGFR5 to varying degrees ranging from moderate to no expression. Some of the giant cells, present in the more mature granulomas, stained quite strongly for FGFR5 whereas the histiocytes of others stained only weakly. Scattered in amongst the granulomas were remnants of lymphoid follicles and granulocytes. The granulocytes stained intensely with the antibody whereas pockets of lymphoid cells expressed lower levels of FGFR5.

The second biopsy was taken from the liver and contained many small inflammatory foci that exhibited a different structure to the archetypal granuloma observed in the first biopsy sample. The liver cells in the second biopsy sample expressed FGFR5 protein. In contrast to the lymph node sample, fewer of the leukocytes expressed high levels of FGFR5 while all of the leukocytes present in a small, presumably emerging, lesion expressed very high levels of FGFR5. These experiments demonstrated that FGFR5 is expressed in granulomas and granulocytes, and may be expressed by some lymphocytes.

In total, the results obtained with these two biopsy samples demonstrate the expression of FGFR5 in sarcoid lesions and indicate that FGFR5 may participate in fuelling the disease process.

Murine Bone

A humerus was collected from an adult mouse, fixed in buffered formalin, embedded in wax, sectioned, and stained for FGFR5 expression. Some, but not all, cells stained for FGFR5. Megakaryocytes, chondrocytes, osteocytes, and stomal cells/osteoblasts all expressed FGFR5, whereas 95% of the small haemopoietic cells did not. It was not possible to identify the 5% of haemopoietic cells expressing FGFR5 based on their morphological characteristics alone.

### EXAMPLE 12

### Identification of FGFR5 Transcripts

cDNA encoding FGFR5 was PCR amplified from 6AVS cells, a bone marrow stromal cell line, and subjected to sequence analysis to confirm that these cells express splice variants of FGFR5. The 6AVS cells express a membrane tethered form of FGFR5 (i.e. it contains a transmembrane domain) but the extracellular domain of the protein was approximately 200 bp shorter than the predicted full-length sequence. This form of FGFR5 is referred to herein as FGFR56. The 200 bp fragment encodes 70 amino acids that form part of the distal region of the second Ig domain, the acid box, CAM (cell adhesion molecule)-binding and heparin binding domains. The resulting receptor encoded by the splice variant created a receptor with an extracellular domain made up of 2 Ig domains linked together with a novel region unlike any other known FGF receptor. The expression of FGFR5δ by bone marrow cells suggests that this transcript plays a role in haemopoiesis. The polynucleotide and amino acid sequences of FGFR5δ are presented herein as SEQ ID NO: 144 and 145, respectively.

### EXAMPLE 13

### Effects of Subcutaneous FGFR5 Administration In Vivo

This Example discloses the effects of in vivo administration of FGFR5β protein to mice.

Experiment 1 used BALB/cByJ mice and experiment 2 used C3H/HeJ mice. Both sets of mice were injected subcutaneously with 5 μg (55 nM in 0.1 ml PBS) of murine FGFR5β extracellular domain (ECD; amino acids 22-373 of SEQ ID NO: 6)—murine IgG3 Fc fusion protein in the morning (prepared as described above) and the same dose in the evening (i.e. each mouse received 10 μg per day) for five days. Control mice received PBS alone. On the sixth day, the mice were sacrificed and the draining lymph nodes (axillary and lateral axillary) were removed. A single cell suspension was generated from the lymph nodes of each mouse and the number of cells collected from each mouse was determine by trypan blue viability counting using a haemocytometer. The lymph node cells collected from the FGFR5-treated mice were then pooled. The lymph node cells collected from the PBS-treated mice were amalgamated into a separate pool of cells. The cells from both the FGFR5 and PBS-treated mice were then stained for the cell surface antigens listed in Table 3, below, and analysed by flow cytometry.

In a third experiment, C3H/HeJ mice were injected subcutaneously with 10 μg (110 nM in 0.1 ml PBS) of murine FGFR5 ECD—human IgG1 Fc fusion protein in one injection per day for 5 days. While the treatment regime differed from that used in Experiments 1 and 2 above, the total dose of protein administered to the mice was not altered. Control mice were administered human IgG1 Fc fragments alone. On the sixth day, the mice were sacrificed and the draining lymph nodes (axillary and lateral axillary) removed. The number of cells collected from each mouse and the presence of cell surface antigens was determined as described above.

As shown in Table 3, in vivo administration of FGFR5 was found to stimulate lymphadenopathy, or enlargement of the lymph nodes. More specifically, administration of FGFR5 was found to result in a preferential increase in the frequency of B cells in the draining lymph nodes. When compared to mice treated with Fc protein, the frequency of B cells doubled in the draining lymph nodes of FGFR5-treated mice. An analysis of the cell cycle state of the B cells by flow cytometry indicated that they were not expanding but were either selectively migrating or being retained in the lymph nodes. This is consistent with the data provided above showing that FGFR5 causes the growth of macrophages but not T or B cells in culture. The cells were, however, activated as there was an increase in the number of cells expressing the very early activation antigen, CD69.

TABLE 3
Comparison of three in vivo experiments testing the effects of
soluble FGFR5 in mice
(The values in this table represent the percentage of total lymph
node cells expressing the indicated marker)

| | | Experiment 1 | | Experiment 2 | | Experiment 3 | |
|---|---|---|---|---|---|---|---|
| | | Balb/c | | C3H/HeJ | | C3H/HeJ | |
| | | Murine | | Murine | | Human | |
| | Cell type | Fc | | Fc | | Fc | Human |
| Markers | recognized | FGFR5 | PBS | FGFR5 | PBS | FGFR5 | Fc |
| CD3 | T cell | 63 | 81 | 59 | 82 | 32 | 67 |
| CD19 | B cell | 35 | 21 | 39 | 16 | 61 | 26 |
| Class II | B cell and | 41 | 20 | ND* | ND | ND | ND |
| | macro- | | | | | | |
| | phage | | | | | | |
| CD45R | B cell | ND | ND | ND | ND | 72 | 31 |
| CD69 | Activated | 23 | 14 | 18 | 10 | 21 | 10 |
| | cells | | | | | | |

*ND = Not determined

Axillary lymph node cells from treated mice were placed in culture and incubated with ³H-thymidine for 18 hours then harvested and analyzed. The cells from the FGFR5-treated mice incorporated more thymidine than the control mice indicating that they were dividing. These studies indicated that FGFR5-induced localized B-cell-dominated lymphadenopathy is caused by localized cellular proliferation.

In order to more accurately target the draining lymph nodes and to monitor the effects of the control and test protein in the same mouse, a footpad injection protocol was utilized. According to this model, the test stimulant was injected under the right hind footpad and the control protein under the left hind footpad. The lymphatic drainage of this site routes to the popliteal lymph nodes. This popliteal lymph node assay was used to assess the effects of treating mice with the murine FGFR5γ-Fc fusion protein.

Groups of four BALB/cByJ mice were injected with 50 μg of FGFR5γ-Fc under the left hind footpad and 50 μg of the control protein FGFR2-Fc under the right hind footpad. In addition, groups of two mice were injected with PBS under the left hind footpad to compare the effects of FGFR5, FGFR2 and PBS. As noted above, the lymphatics from this site drain to the popliteal lymph node. These lymph nodes were collected 1, 2 and 3 days after the initiation of the experiment. The cells from each node were released and counted using a haemocytometer, and their viability assessed by the Trypan blue exclusion assay. The cells from the individual nodes were then stained with fluorescently labeled antibodies and the relative frequencies of each of the major haemopoietic cell types assessed by flow cytometry.

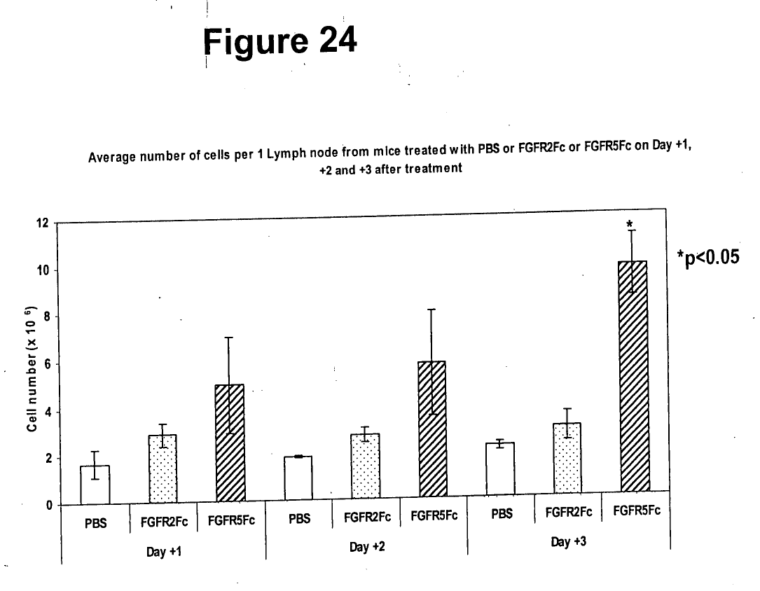

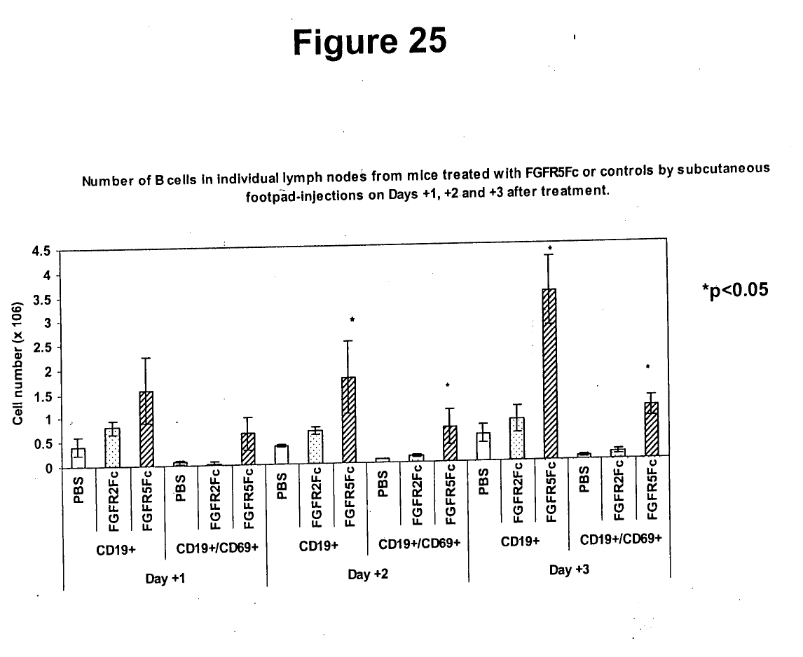

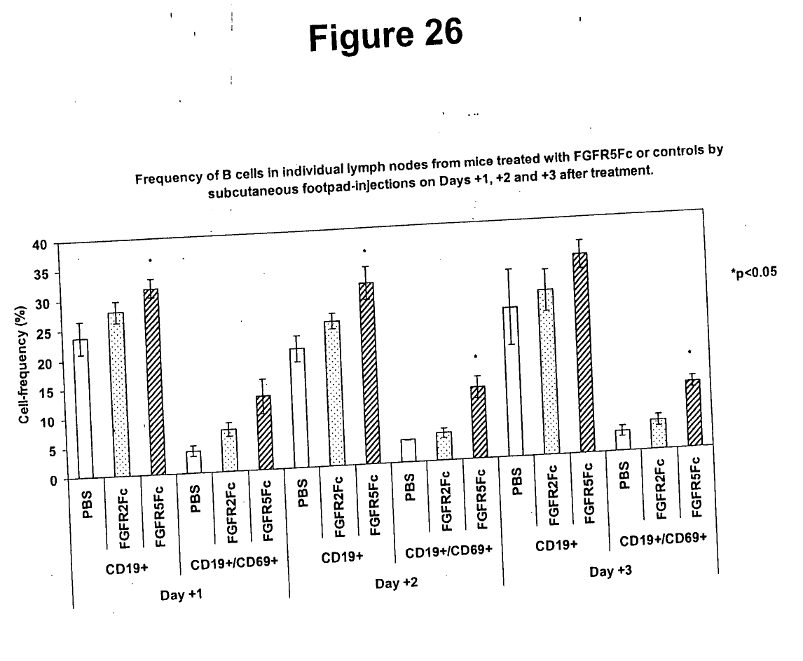

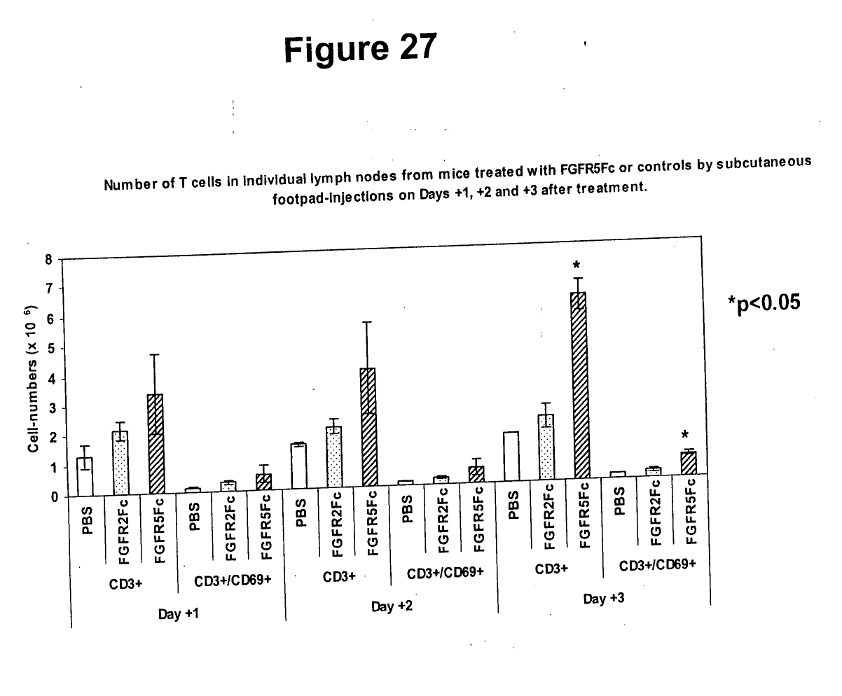

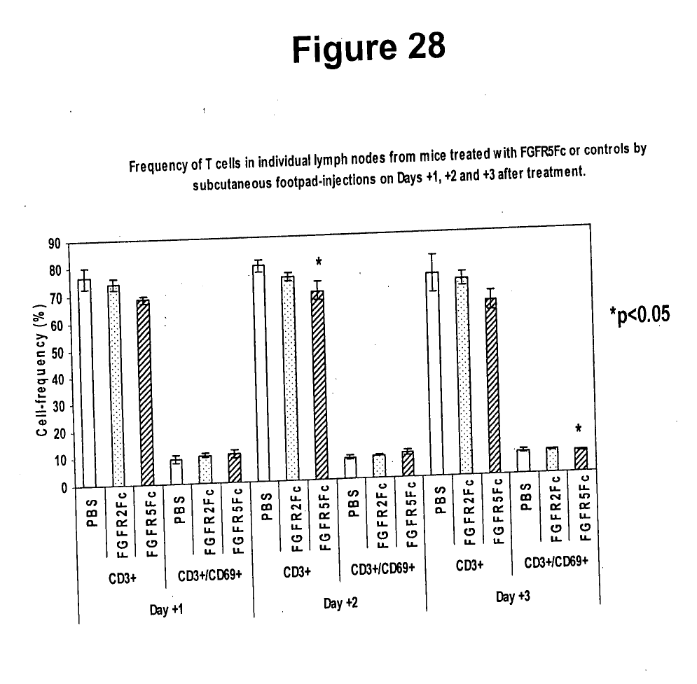

The results of these assays are shown in FIGS. 24-28. Specifically, FIG. 24 shows that subcutaneous administration of FGFR5γ-Fc was found to induce a localized lympadenopathy in the draining popliteal lymph nodes. More specifically, FGFR5γ-Fc induced an increase in the total number of cells isolated from the popliteal lymph nodes that was apparent 24 hrs after the protein had been administered and rose to 3.2 times the number of cells isolated from the nodes draining the FGFR2 injection site. The data provided in FIG. 25 demonstrates that subcutaneous administration of FGFR5γ-Fc induced a statistically significant increase in the numbers of B cells (CD19+) and activated B cells (CD19+CD69+) 2 and 3 days after treatment with FGFR5γ-Fc and FGFR2-Fc fusion proteins. FIG. 26 shows that subcutaneous administration of FGFR5γ-Fc induced a statistically significant increase in the frequency of B cells (CD19+) and activated B cells (CD19+CD69+) 2 and 3 days after treatment with the FGFR5γ and FGFR2-Fc fusion proteins. FIG. 27 shows that subcutaneous administration of FGFR5γ-Fc induced a statistically significant increase in the numbers of T cells (CD3+) and activated T cells (CD3+CD69+) 3 days after treatment with the FGFR5γ and FGFR2-Fc fusion proteins. FIG. 28 shows that subcutaneous administration of FGFR5γ-Fc induced a decrease in the frequency of T cells (CD3+) 2 days after treatment and activated T cells (CD3+CD69+) 3 days after treatment with the FGFR5γ and FGFR2-Fc fusion proteins. In FIGS. 24-28, the columns marked with an asterisk denote an FGFR5γ-Fc treatment group that differs significantly (p<0.05) from the FGFR2-Fc controls as assessed by the students T test.

These experiments demonstrate that FGFR5 induced a localized B cell dominated lymphadenopathy, as shown by an increase in the total number of cells extracted from the lymph node and a preferential increase in both the number and percentage of activated B cells (CD19+CD69+cells). All of the FGFR5 induced changes were most apparent 3 days after treatment. Although the frequency of T cells declined in the lymph nodes collected from the FGFR5 treated mice, the absolute number of T cells per node increased. These data show that FGFR5 activates the immune system and therefore has the ability to augment responses to antigens in an adjuvant-like manner.

### EXAMPLE 14

### Effect of FGFR5 on Bone Marrow Growth and Differentiation

This Example discloses the effects of FGFR5 on haemopoiesis through stimulation of murine bone marrow cells (BMC).

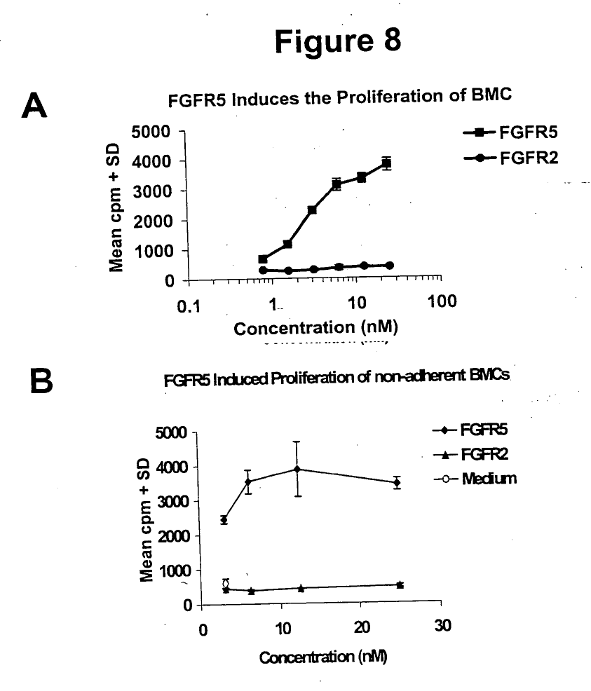

The effect of FGFR5-Fc on bone marrow growth was assessed in a standard tritiated thymidine proliferation assay. Briefly, murine bone marrow cells were collected from the humerus and resuspended in DMEM supplemented with 5% FBS, 2mM L-glutamine (Sigma, St Louis Mo.), 1 mM sodium pyruvate (Life Technologies, Gibco BRL, Gaithersburg Md.), 0.77 mM L-asparagine (Sigma), 0.2 mM arginine (Sigma), 160 mM penicillin G (Sigma), 70 mM dihydrostreptomycin sulfate (Boehringer Mannheim, Roche Molecular Biochemicals, Basel, Switzerland) at 2×10⁶ cells/ml. The cells were seeded into 96 well round bottom plates in 0.1 ml of media and various concentrations of FGFR5-Fc, FGFR2-Fc, IL-7 or media added to the plates in 0.1 ml media. The cultures were then incubated at 37° C. in a humidified atmosphere containing 10% CO₂ in air for 3 days. Tritiated thymidine was added to the cultures for the final 16 hrs and cells harvested onto glass fiber filters and thymidine incorporation quantified by standard liquid scintillation counting. FIG. 8A shows that FGFR5 induced a dose dependent proliferation of murine bone marrow cells.

Bone marrow contains numerous haemopoietic cell types at various stages of differentiation and therefore FGFR5 may stimulate the growth of one or many of these cell types. The following experiments were performed to determine which cells grew in response to FGFR5-Fc stimulation.

The effect of FGFR5 on the proliferation of non-adherent BMCs is presented in FIG. 8B. Murine bone marrow cells were isolated from 6-8 week old female Balb/c mice. Adherent BMCs were prepared by inoculating cells into 96-well plates at 1×10⁶ cells/well, incubating at 37° C. for 3 hours and then removing non-adherent cells. The non-adherent BMCs were harvested after incubating BMCs in culture dishes at 37° C. for 3 hours to remove adherent cells and then seeded into a 96-well plate at 2×10⁶ cells/well. The mean cell proliferation in the presence of varying concentrations of FGFR5, FGFR2 or Medium control was measured from the incorporation of tritiated thymidine. Data represent mean cpm±SD.

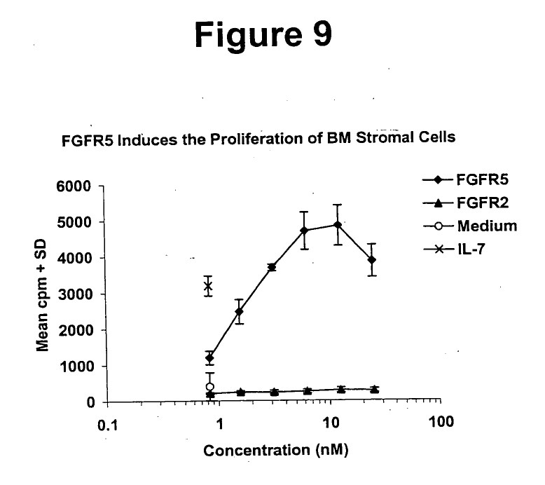

The effect of FGFR5 on the proliferation of aggregated (stromal cell enriched) BMCs is presented in FIG. 9. Aggregated BMCs were prepared as described previously (Parkin et al., J. Immunol. 169:2292-2302 (2002) and distributed into 96-well plates at 5.5×10⁴ cells/well. The mean cell proliferation in the presence of varying concentrations of FGFR5, FGFR2 or medium control and IL-7 (10 ng/ml) was measured from the incorporation of tritiated thymidine. Data represent mean cpm±SD.

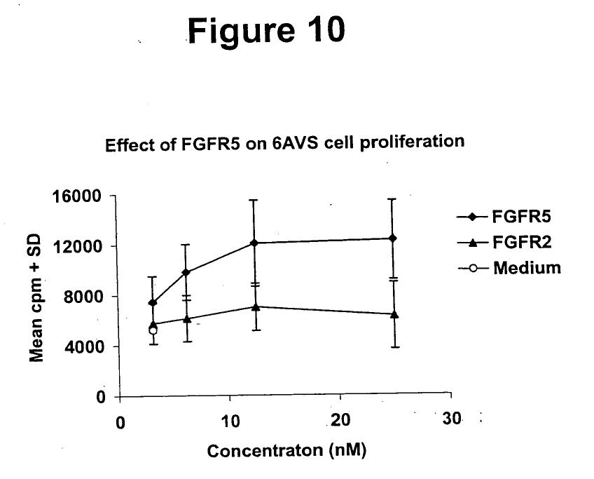

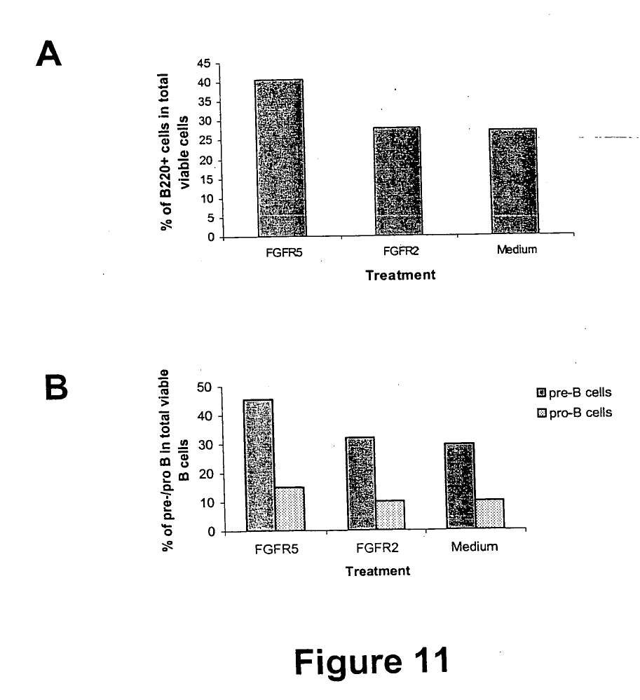

The effect of FGFR5 on proliferation of the murine bone marrow cell line 6AVS is presented in FIG. 10. 6AVS cells (2×10³ cells/well) were seeded into 96-well plates, in DMEM supplemented with 0.05% FBS and incubated with varying concentrations of FGFR5 or FGFR2 in a humidified incubator at 37° C. and 5% CO₂ in air. [³H]-thymidine incorporation levels were assessed at day 3, after a 16 hour pulse. The data are presented as mean cpm±SD of triplicate wells.

The non-adherent bone marrow cells proliferating in response to FGFR5 stimulation were identified by flow cytometry. Bone marrow cells were distributed into 6-well plates (2×10⁶/ml, 3 ml/well) with or without FGFR5 (25 nM) or FGFR2 (25 nM). After incubating at 37° C., 5% CO₂, for 3 days, the surface phenotype of the cells was determined with immunofluorescence labeling. FGFR5 stimulates the preferential expansion of pre-B cells in culture as illustrated in FIGS. 1A (% of B220+ cells in total viable cells) and 11B (% of pre/pro-B in total viable B cells).

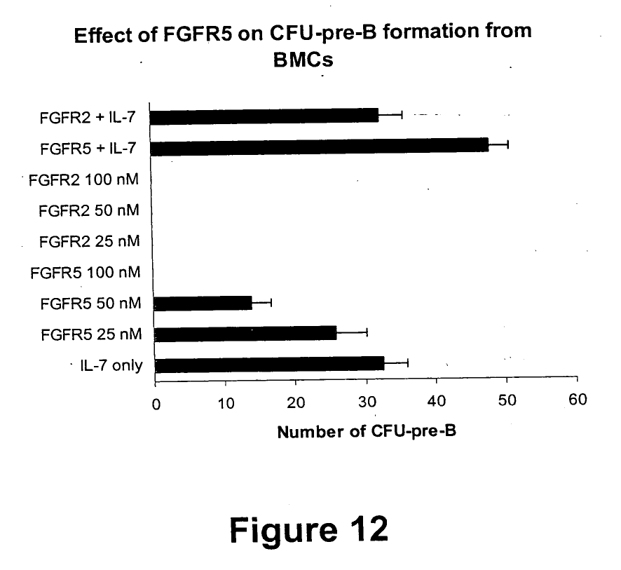

B-cell colony formation assays were utilized to determine whether FGFR5 had a direct effect on B-cell development. The effect of FGFR5 on CFU-pre-B formation from BMC is presented in FIG. 12. Bone marrow cells (5×10⁴) in 1 ml of complete IMDM media containing either 10 ng/ml IL-7, the indicated amount of FGFR5/FGFR2, or a combination of 25 nM FGFR5/FGFR2 and 10 ng/ml IL-7, were plated in 35-mm culture dishes and incubated at 37° C., 5% CO₂. Complete media consisted of IMDM, 1% methylcellulose, 30% FBS, 10⁻⁴ M 2-mercaptoethanol, 2 mM L-glutamine, 100 U/ml penicillin and 100 μg/ml streptomycin. Colonies comprising >30 cells were quantified after 7 days. Data represent mean cpm±SD from duplicate cultures.

After 10 days of culture, the colonies were counted. There were no colonies detected in either the media or FGFR2-stimulated cultures whereas FGFR5 and IL-7 stimulated growth of equivalent numbers of colonies. These results demonstrated that FGFR5 and IL-7 had an additive effect, indicating that FGFR5 and IL-7 triggered complimentary, but distinct, growth and development signals.

Colonies formed following FGFR5 stimulation had a similar appearance to the pre-B cells colonies induced by IL-7. These data indicated that each colony arose from one responsive precursor cell, and that IL-7 and FGFR5 had a direct effect on the cells—not via any accessory cells that are spatially separated from the responders in the gelatinous media. These data also demonstrated that FGFR5 stimulated the formation of pre-B cells from BMC cultures.

Treatment with either FGFR5 or IL-7 induced growth of B cells as all expressed CD45R (B220). However FGFR5 stimulated the growth of cells with a more mature B cell phenotype. The FGFR5-stimulated cells contained 33% IgM+ B cells whereas only 10% of the cells generated in the IL-7 cultures were of this phenotype. In accordance with this observation, the FGFR5 colonies appeared to be smaller on average than the IL-7 colonies, indicating that FGFR5 stimulated cells of a more mature phenotype. The effects of FGFR5 appeared to mimic those of thymic stromal-derived lymphopoietin (TSLP) which stimulates B-cell colony formation in these assays and preferentially induces growth of B220+ IgM+ B-cells.

### EXAMPLE 15

### Effect of Monomeric, Dimeric, and Tetrameric FGFR5 on Adherent Peripheral Blood Mononuclear Cell (PBMC) and Anti-CD3 Induced PBMC Growth

This Example demonstrates that the murine anti-FGFR5 monoclonal antibody 15G6, enhances the activity of the FGFR5 by crosslinking either the dimeric FGFR5-Fc fusion protein or monomeric FGFR5.

Monoclonal antibodies were generated to the recombinant murine FGFR5β ECD by standard techniques described in the literature. Briefly, four mice were immunized with murine FGFR5 extracellular domain (ECD) fused to the murine IgG3 Fc. Serum samples collected from the mice were tested for antibodies reactive to murine FGFR5. Two of the four mice were confirmed to produce anti-FGFR5 antibodies. A single mouse having the highest titer of FGFR5 antibodies was reimmunized with the FGFR5-Fc fusion protein. Splenocytes were isolated from this mouse and standard methods were employed to fuse the splenocytes to myeloma cells to generate hybridomas. After the fusion, the cells were dispensed into eighteen 96-well plates and cultured in media to select for hybridomas. 700 independent hybridoma lines were screened for FGFR5-reactive antibodies using the murine FGFR5β ECD fused to human IgG Fc in an ELISA assay. Three independent, positive hybidomas were identified and further screened for FGFR5-specific antibodies using murine FGFR1-4 human IgG Fc fusion proteins. The hybridomas specific for FGFR5 were subcloned, supernatants generated, and antibodies purified for use in the following assays.

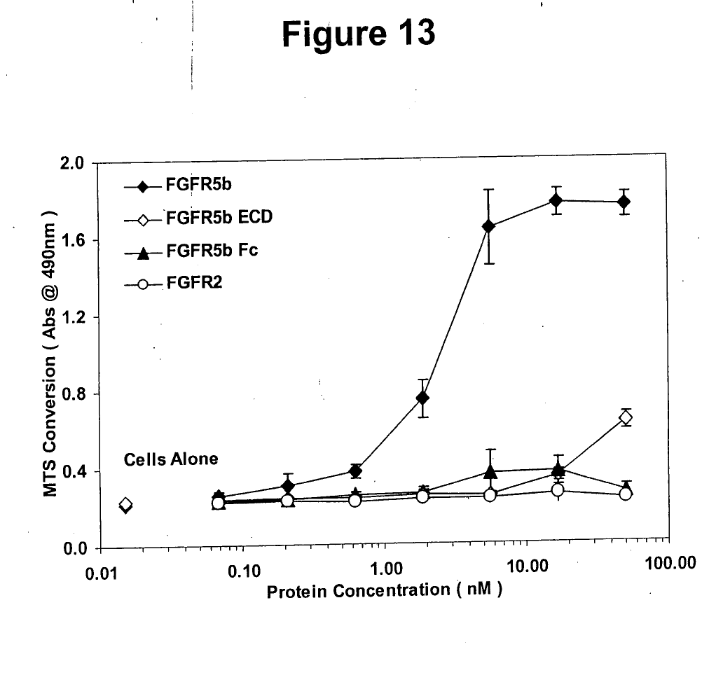

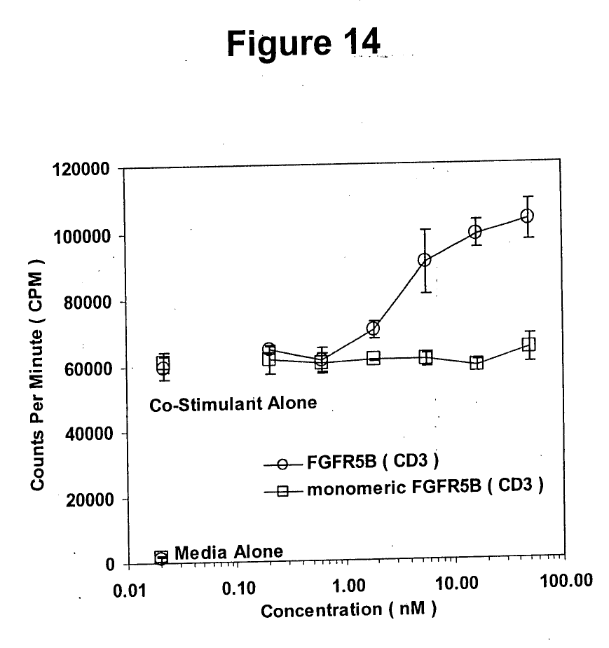

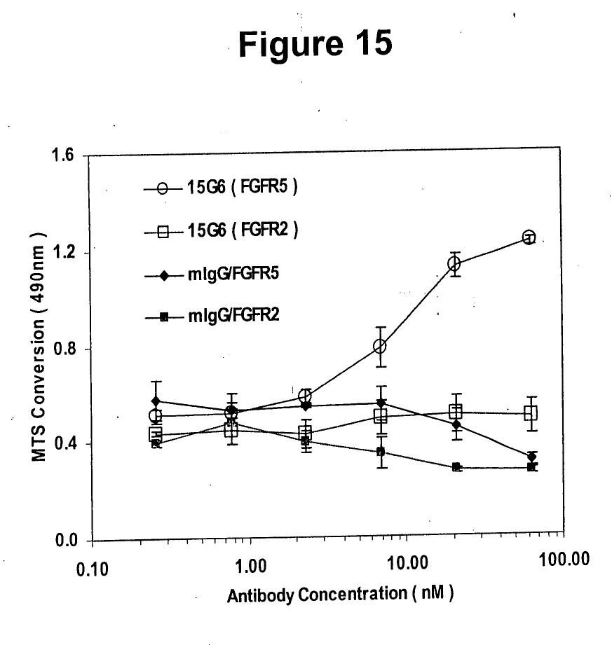

Monomeric FGFR5 was generated by cleaving the Fc region from the FGFR5-Fc fusion protein such that a 55 kDa FGFR5 extracellular domain was released. This protein was tested in assays and showed 100-fold less activity in either of the standard human PBMC assays routinely used to test the biological effects of FGFR5 (FIGS. 13 and 14). Dimerization of FGFR5-Fc to form tetramers augmented the ability of FGFR5-Fc to stimulate the growth of adherent PBMC (FIG. 15).

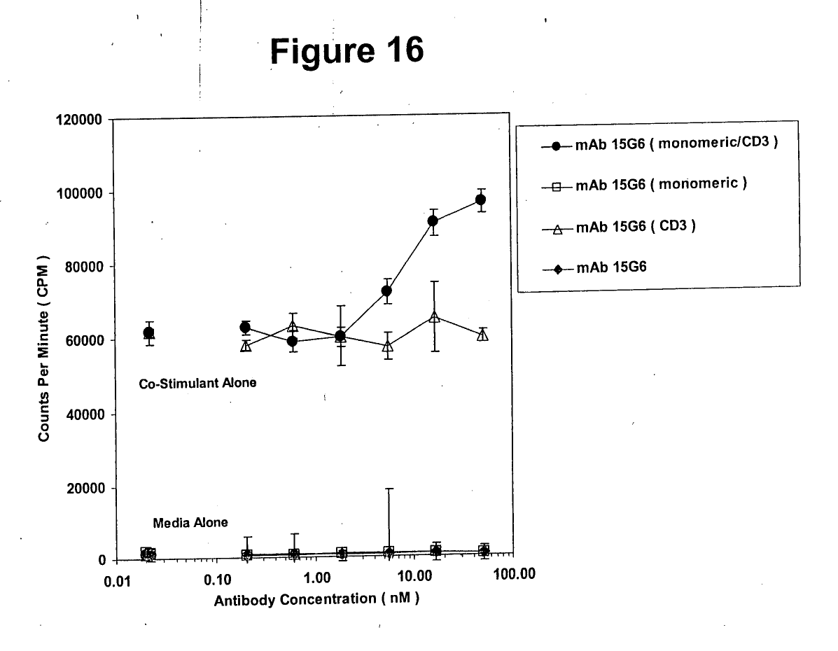

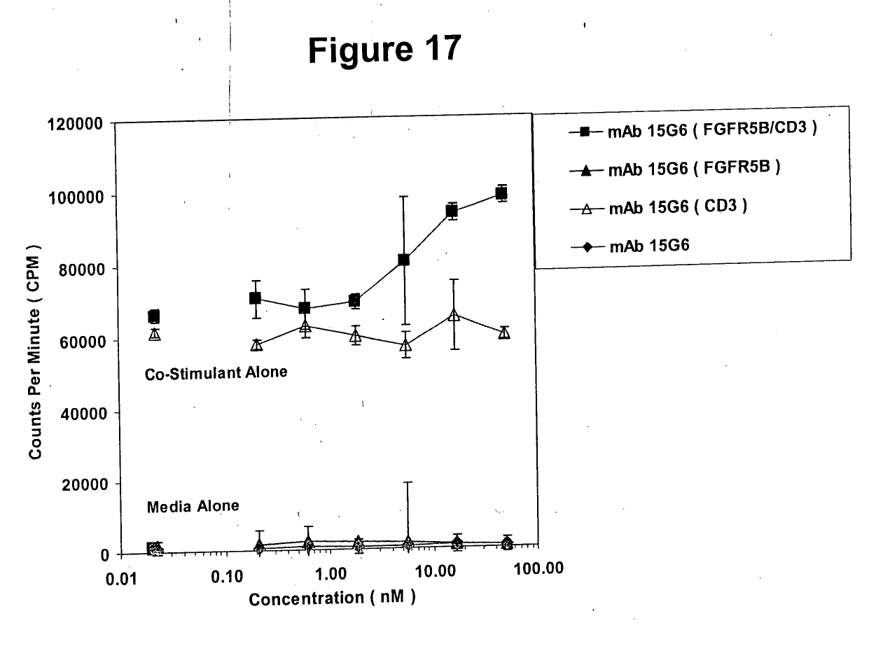

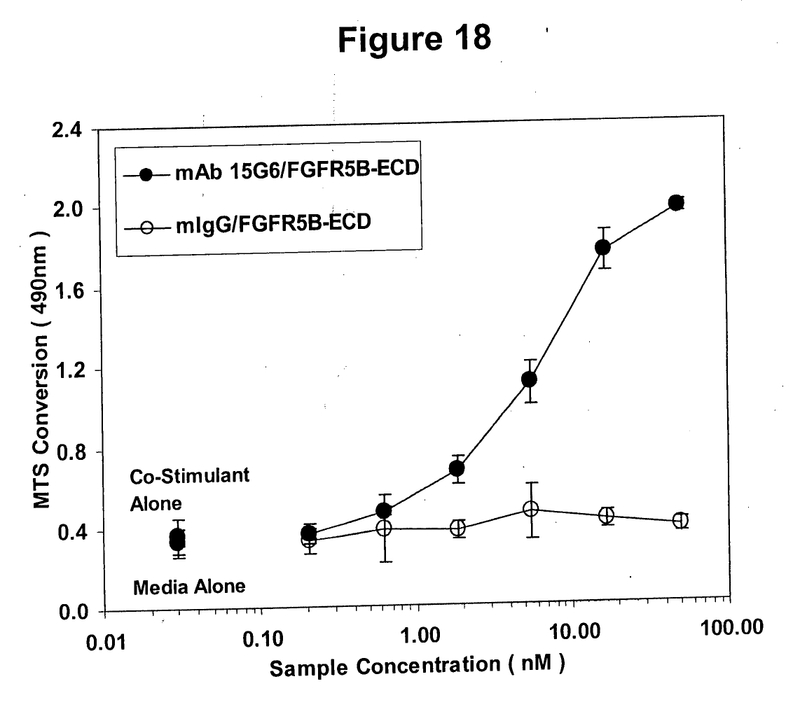

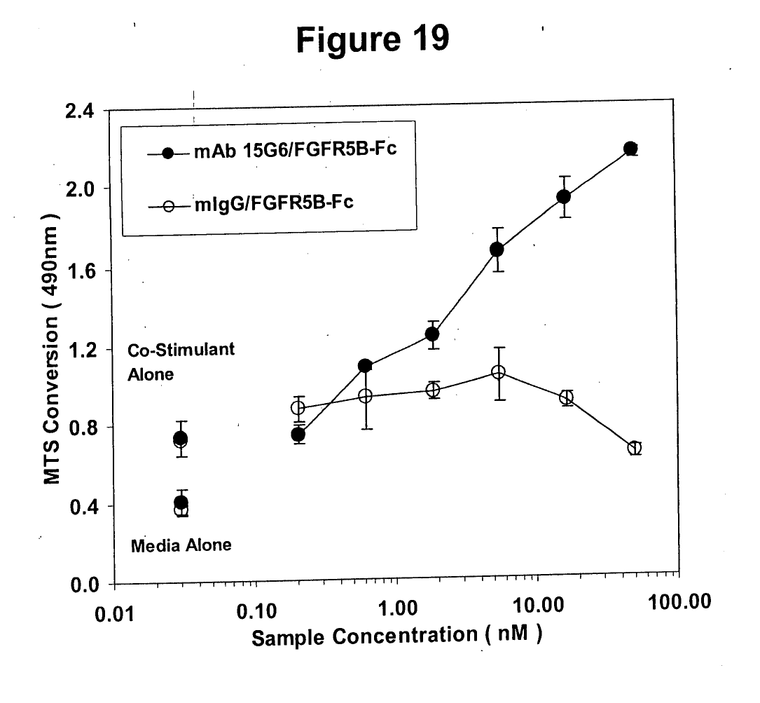

The monoclonal antibody to FGFR5 was capable of dimerizing the monomer thereby recovering its activity. While monomeric FGFR5 was incapable of augmenting anti-CD3 stimulated PBMC proliferation (FIG. 14), the dimerized monomeric FGFR5 augmented the growth of anti-CD3 induced PBMC proliferation in a similar manner as the dimeric FGFR5-Fc fusion protein (FIG. 16). Furthermore, dimerized FGFR5-Fc (i.e. tetrameric FGFR5-Fc) augmented the anti-CD3 induced growth of human PBMC (FIG. 17). In a similar fashion, the FGFR5-specific monoclonal antibody enhanced the activity of the monomeric FGFR5 and dimeric FGFR5-Fc fusion protein in the PBMC adherence assay (FIGS. 18 and 19).

In total, these data demonstrate that multimerization of FGFR5 enhanced its activity. Without wishing to be limited to any specific mechanism of action, these data indicate that a cell-associated form of FGFR5 may be more potent than a naturally occurring soluble version of the protein unless the soluble FGFR5 is first polymerized by, for example, attachment to a scaffold, such as one or more extracellular matrix proteins.

### EXAMPLE 16

### Heparin is an FGFR5-Binding Molecule and Inhibitor of FGFR5 Function

Many studies have shown that fibroblast growth factors bind to their receptors in the context of heparin-like glycosaminoglycans (HLGAG). Both FGFs and their receptors are heparin-binding proteins and the three components, FGF, FGFR and HLGAG, form a complex and induce signalling. A series of experiments was performed to determine whether FGFR5 is a heparin-binding protein and whether heparin alters the effects of FGFR5 on the immune system. The heparin-binding abilities of FGFR5 were tested chromatographically. FGFR5 was run onto a heparin Hi-Trap affinity column (Amersham Pharmacia Biotech) and the bound protein eluted with a salt gradient.

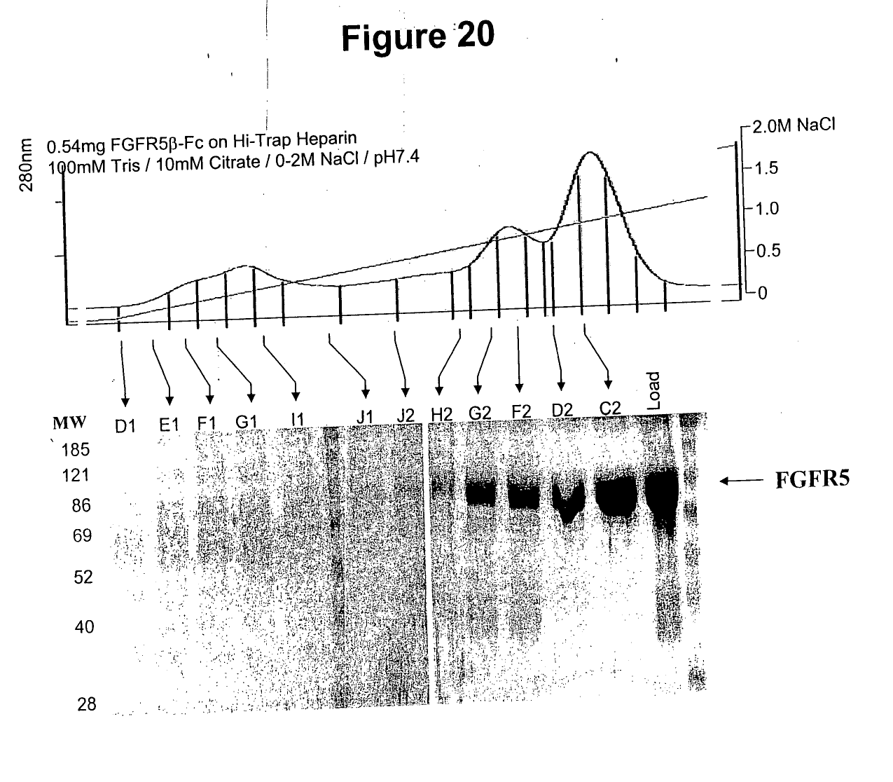

FIG. 20 shows that FGFR5 bound to heparin and that the majority of the protein was eluted with ^{˜}1 M NaCl. Analysis of the proteins eluted from the column on SDS-PAGE gels confirmed that FGFR5 eluted from the column at this salt concentration.

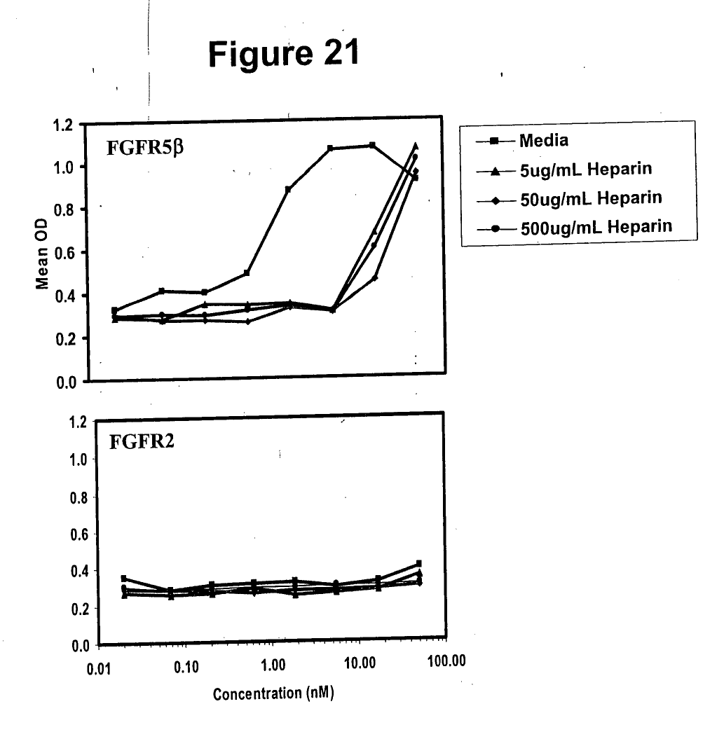

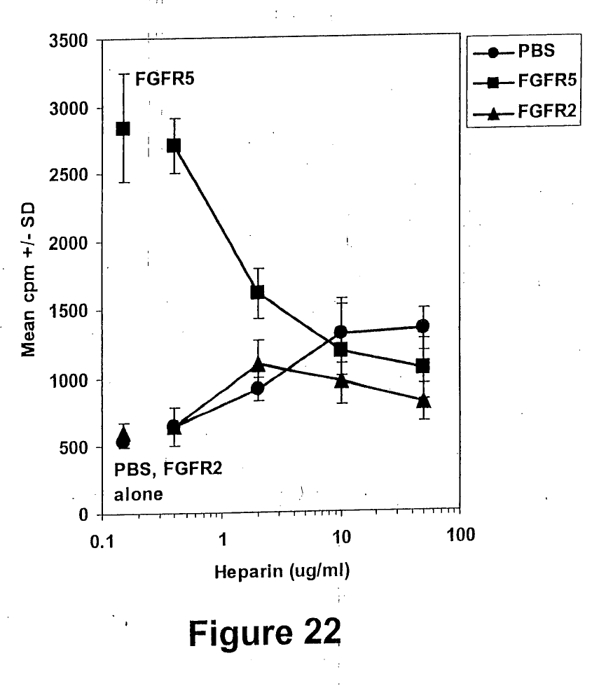

Heparin was added to the macrophage adherence assay to determine whether it would influence the ability of FGFR5 to stimulate the growth of adherent PBMC. As shown in FIG. 21, heparin inhibited the function of FGFR5 at a concentration of 5 ug/ml. Furthermore, heparin sulphate inhibited the murine FGFR5β-Fc induced proliferation of murine bone marrow cells in a dose dependent manner (FIG. 22). These results indicate that heparin blocks the ligand binding portion of FGFR5, that the heparin-binding domain of FGFR5 is involved in the binding of the cognate ligand responsible for the functions of FGFR5, that the ligand may be a HLGAG, and that heparin or heparin-like molecules may serve as inhibitors of FGFR5 function.

### EXAMPLE 17

### Effects of Intravenous FGFR5 Administration In Vivo

This Example shows the effects of in vivo intravenous administration of FGFR5-Fc on up-regulation of cell-surface marker expression and frequency of pre-B cells.

Mice were treated with 100 μg of either FGFR5-Fc or FGFR2-Fc intravenously on day 1 and 4 of the experiment (200 μg total/mouse). The mice were euthanized, and the bone marrow and spleens collected for analysis on day 8. The cells released from each of these organs were counted, stained for a panel of surface markers, and analyzed by flow cytometry (FACS).

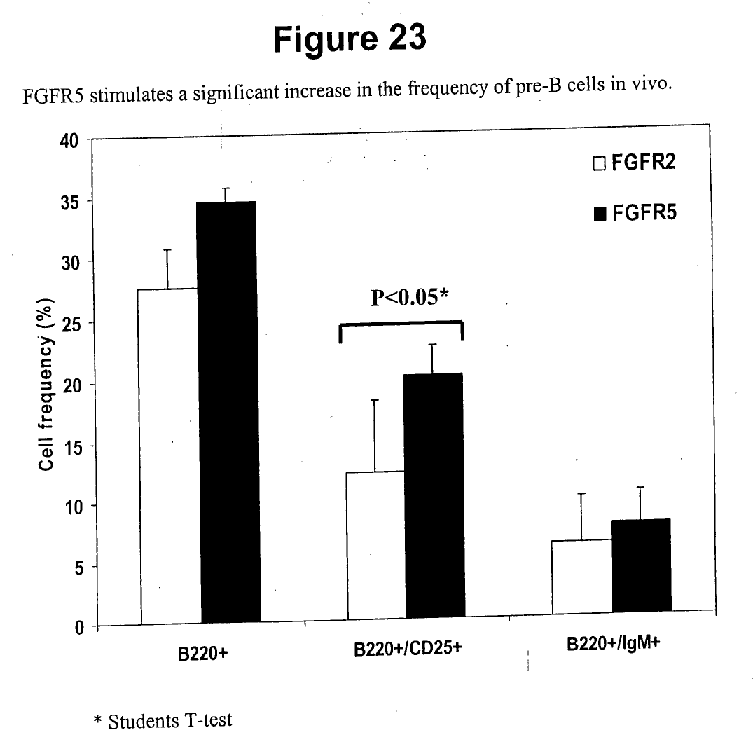

There were no statistically significant differences in the numbers of cells collected from the organs or their viability but there were FGFR5-related changes to the frequency of B cell subsets in the bone marrow. As shown in FIG. 23, FGFR5-Fc induced a statistically significant increase in the percentage of pre-B cells (B220⁺CD25⁺) in the bone marrow whereas there was little effect on the immature B cells (B220⁺IgM⁺). The results shown are representative of two experiments that yielded similar results. These results are consistent with data demonstrating that FGFR5 drives the expansion of a pre-B cell population in murine bone marrow cultures.

### EXAMPLE 18

### Effects of Intraperitoneal Administration of FGFR5

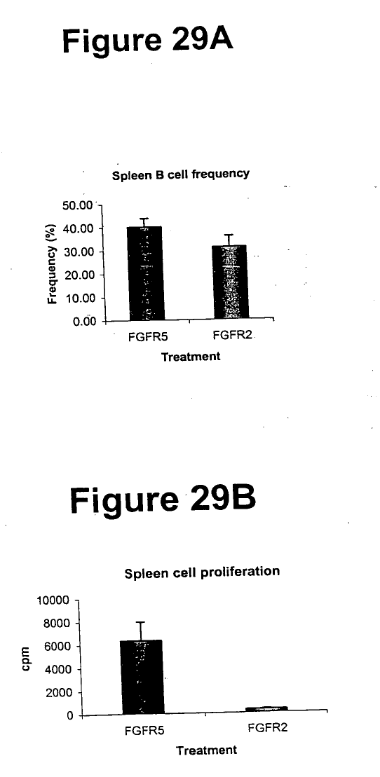

The effects of FGFR5 on the murine immune system were determined by the intraperitoneal administration of FGFR5β and the control protein, FGFR2. Two groups of four BALB/c mice (6-8 weeks old) were treated intraperitoneally (i.p.) with 200 μg of murine FGFR5β human Fc or human FGFR2 human Fc recombinant proteins (in 0.20 ml PBS) on day 1, day 3 and day 5. On day 7, mice were humanely euthanazed using CO₂, and bone marrow, spleen, peritoneal cells and the draining lymph node (posterior mediastinal lymph node) were removed for analysis of any alterations in cell number and cellularity. Mice treated with FGFR5β showed a significant increase in spleen size and total cell number. Phenotypic analysis by flow cytometry revealed a 33% increase in B cell frequency compared with FGFR2-treated mice (FIG. 29A). This increase was at least partly attributed to an elevated cell proliferation. This notion was based on the observation that the spontaneous proliferation rate, determined by in vitro short-term (24 h) culture of spleen cells and ³H-thymidine incorporation of these cells, was markedly higher in splenocytes from FGFR5β-treated mice than that from FGFR2-treated mice (FIG. 29B). There were no obvious differences in the frequency of other cell lineages between FGFR5β and FGFR2-treated animals.

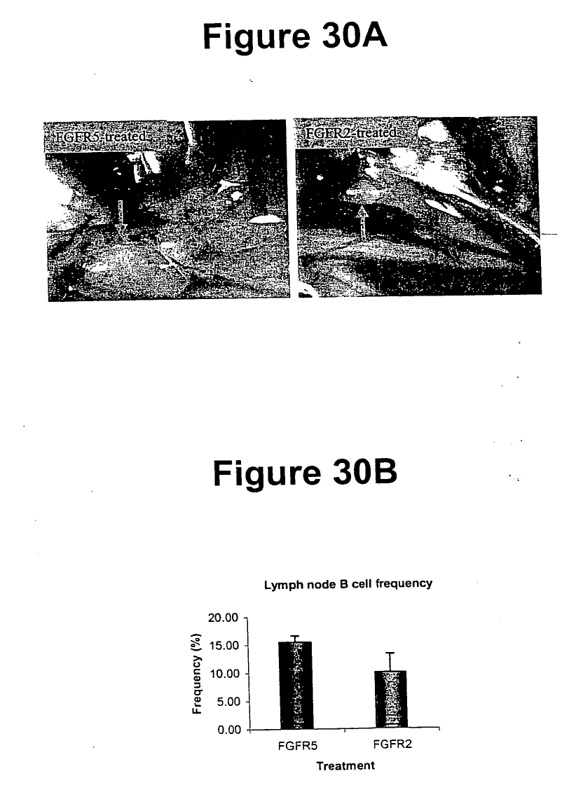

FGFR5β treatment resulted in the lymphadenopathy of the draining posterior mediastinal lymph node (FIG. 30A), a two-fold increase in total cell number, and preferential enhancement of B cell frequency (FIG. 30B). No major changes in the frequency of other lineages of cells were observed.

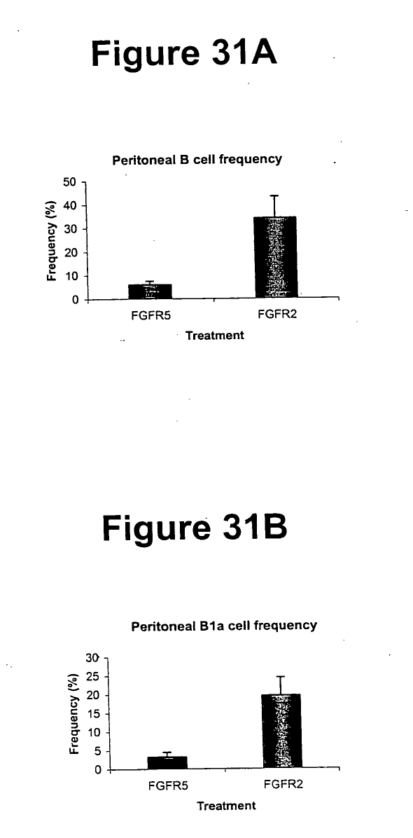

FGFR5β treatment also caused an increase in the number of cells harvested from the peritoneal cavity. Phenotypic analysis of the peritoneal cells revealed that FGFR5 reduced B cell frequency from 35% to about 5% (FIG. 31A), while no changes were seen in the incidence of other cell lineages, such as T cells and macrophages. This marked reduction mainly resulted from the decrease of CD5+ B1a cells, the predominant B cell population in the peritoneal cavity, whose frequency was decreased from 20% to about 3% by FGFR5β treatment (FIG. 31B). This dramatic reduction was not due to the programmed cell death (apoptosis) as demonstrated by Annexin V staining of the cells (data not shown).

Intraperitoneal administration of FGFR5β slightly decreased the frequency of mature B cells in bone marrow but had no effect in the incidence of pre-B cells (data not shown). No changes were seen in the incidence of other cell lineages. Unlike its effects in spleen, i.p injection of FGFR5β had no effects on bone marrow cell number or their ability to proliferate in culture.

Our previous studies, described in Example 15, showed that subcutaneous injection of soluble FGFR5β induces lymphadenopathy and expansion of B cells in the peripheral lymph nodes. The results reported here are in agreement with the previous observations and provide further evidence that FGFR5 is a B cell stimulator. B cells are immune cells responsible for antibody production and a key cell population involved in the development of autoimmunity and autoimmune diseases. It has been established that the exaggeration of B cell number and any of its functions, such as activation, proliferation, migration, signaling, cytokine production, antibody production and costimulation factor expression, could contribute to the development of autoimmunity (Criscione et al., Curr. Rheumatol. 5:264-269 (2003); Lampe et al., J. Immunol. 147:2902-2906 (1991); Klinman et al., J. Exp. Med. 165:1755-1760 (1987)). The potentiation of B cell expansion and proliferation in peripheral lymphoid tissues, such as spleen and lymph node, by FGFR5β indicates that this molecule may contribute to the development and progression of autoimmune diseases, such as SLE, and that antagonists of it may have therapeutic potential.

The i.p administration of FGFR5β caused a marked reduction of peritoneal CD5⁺ B1a cells, which was unlikely to be caused by apoptotic cell death. However, it was possible that FGFR5β stimulated the migration of peritoneal B1a cells to other peripheral tissues. In these experiments, the frequency of B1a cells was only determined in spleen and the draining lymph node, which showed a slight increase of this population in FGFR5β-treated mice compared with FGFR2-treated mice (data not shown). B1a cells are thought to be involved in the pathogenesis of autoimmune diseases by producing pathogenic autoantibodies. It has been suggested that increase of B1a cell number and its migration to peripheral tissues are associated with the development of autoimmune diseases (Ito et al., J. Immunol. 172:3628-3634 (2004)). B1a cell infiltration in spleen, lymph node and other tissues has been reported in mouse models of autoimmune diseases, and is thought to contribute to the production of autoantibodies, deposition of immune complex and tissue damage. The effect of FGFR5 on B1a cells provides another potential link between this molecule and autoimmune diseases.

### EXAMPLE 19

### Analysis of Spleen Cells Extracted From FGFR5-Treated Mice

Spleen cells were collected from mice injected intraperitoneally with either FGFR5β or FGFR2 for 1 week as described in Example 21. The cells were cultured in vitro in RPMI 1640 supplemented with 10% heat-inactivated FBS, 5×10⁻⁵ M 2-mercaptoethanol (2-ME), 1 mM sodium pyruvate, 2 mM L-glutamine, 100 U/ml penicillin and 100 μg/ml streptomycin. Cells from FGFR2-treated animals died rapidly and after 5 days in culture almost all cells from FGFR2-treated mice were dead, whereas about 70% of splenocytes from FGFR5β-treated mice remained viable. After 4 weeks, there were still some viable cells seen in the cultures. In the first few days of culture, the cells isolated from animals treated with FGFR5β proliferated strongly as determined by ³H-thymidine incorporation but the proliferation rate declined as the cultures aged. This phenomenon was been observed in two independent experiments.

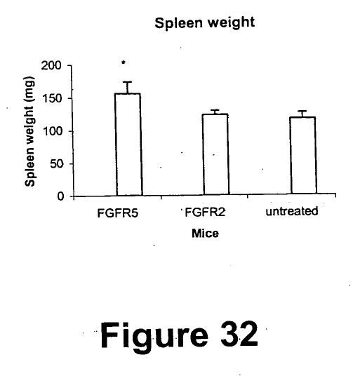

An in-depth analysis of this phenomenon was performed on spleen cells extracted from 6-8 week old BALB/c mice that were treated with FGFR5β or FGFR2 for 3 weeks. The mice received 200 μg of recombinant protein intraperitoneally 3 times per week in 0.2 ml of PBS. Mice were humanely euthanazed using CO₂ 2 days after the last injection and spleen was removed for analysis. As observed in the 1-week treatment regime, mice treated with FGFR5β showed a significant increase in spleen size. The spleens from the FGFR5β-treated mice were on average 30% larger than those from the mice treated with FGFR2 or from untreated mice (FIG. 32).

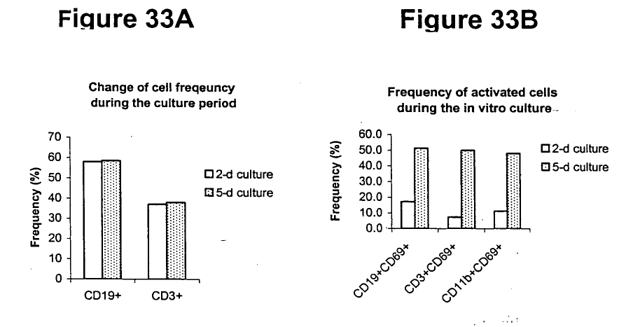

Spleen cells were isolated and cultured as described above. The spleen cell cultures established from the mice treated with FGFR5β for 3 weeks were similar to those previously described from mice treated with FGFR5β for 1 week except that they appeared to proliferate at a greater rate (data not shown). The splenocytes isolated from FGFR2-treated mice died rapidly in culture and therefore most of the analysis described was performed on the cultures derived from FGFR5β-treated animals. Phenotypic analysis of the cells revealed that B and T lymphocytes accounted for approximately 95% of the cells over the first 5 days of culture. Interestingly, the relative frequency of the cells did not change over this period, indicating that both populations of lymphocytes were proliferating at a similar rate (FIG. 33A). CD69, an early activation marker, was used to determine cell activation state. As shown in FIG. 33B, a high percentage of the cultured cells (B, T and CD11b⁺ cells) were activated and the incidence of activated cells increased during the period of 2 to 5 days of culture.

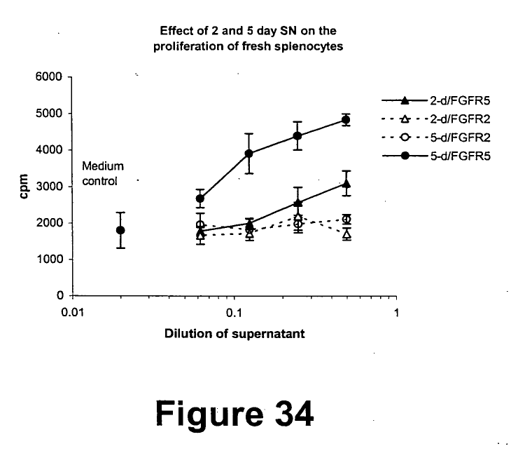

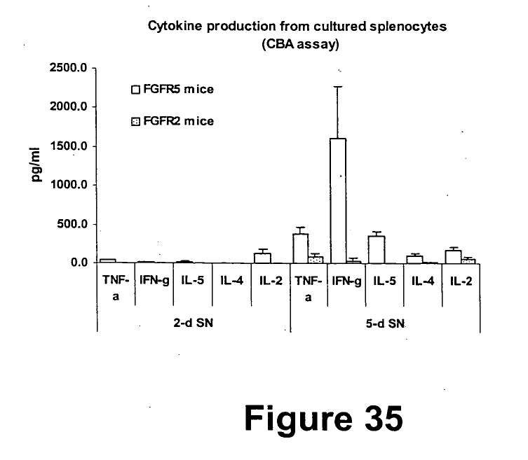

Supernatants (SN) were collected from the cultures 2 and 5 days after initiation and the levels of some cytokines were determined using the TH1/TH2 Cytokine Bead Array (CBA) Kit (BD BioSciences). Only low levels of cytokines were detected in the 2 day supernatants whereas much higher levels were found in the supernatants from the FGFR5β cultures after 5 days. These supernatants were tested for the presence of growth factors in a simple murine splenocyte growth assay. The supernatants were diluted in media and spleen cells isolated from untreated BALB/c mice were added to the cultures. The cells were cultured for 3 days in a microtiter plate and ³H-thymidine added to the wells for the last 16 hrs of the culture. The cells were harvested onto glass fiber filter paper and the ³H-thymidine incorporation measured by standard liquid scintillation counting. The supernatants from the spleen cells cultured from the FGFR5β-treated mice induced the proliferation of the naïve spleen cells in a dose dependent manner whereas the supernatant collected from the FGFR2 cultures had no influence on the assay (FIG. 34). The supernatants collected after 5 days of culture were significantly more potent than the 2 day supernatant. This result indicates that the spleen cells extracted from the FGFR5β-treated mice produce a growth factor, or factors, for naïve splenocytes and that it accumulates in the culture media for at least a 5 day period. This is an unusual observation as most growth factors will not induce the proliferation of unactivated spleen cells and, although the supernatants do contain known growth factors such as IL-2, IL-4 and IL-5 (FIG. 35), the concentrations detected in the supernatant would not appear to be sufficient to drive the levels of proliferation observed in this assay. Collectively these data suggest that the supernatants contain an as yet unidentified growth factor or factors for splenocytes.

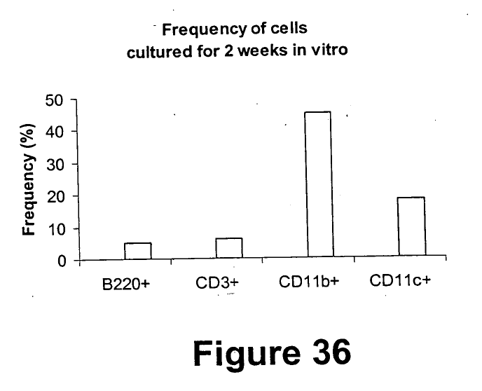

The spleen cell cultures derived from the FGFR5β-treated mice continued to survive for at least 3 weeks and an analysis of the cells, which are predominantly non-adherent, after 2 weeks in culture revealed that the frequency of lymphocytes had declined dramatically. After 2 weeks in culture approximately 10% of the cells are lymphocytes and most (approximately 50%) express either low or intermediate levels of the CD11b marker (FIG. 36). Many of the CD11b intermediate cells also express CD11c and MHC class II (data not shown). Cells with this phenotype are dendritic cells and they account for 20% of the cells in the culture. Many more of the cells express CD11b and MHC class II but do not express CD11c (approximately a further 20%). It is possible that these are also dendritic cells but further phenotypic analysis will be required to determine this. These data suggest that dendritic cells are being generated in the culture without the addition of any growth factors or other stimulants. The treatment of the mice with FGFR5β must alter the cellular composition of the spleen in such a way that it creates an environment where sustained lymphoproliferation and dendritic cell development can occur when the cells are placed in culture without the addition of extra stimulants. It is possible that the two phenomena are linked and that the development of dendritic cells in culture drives the proliferation of the lymphocytes through a combination of membrane bound and secreted factors.

### EXAMPLE 20

### FGFR5 Administration In Vivo Leads to Increased Ig Levels and Production of Autoantibodies

The observations described in Examples 21 and 22 above, indicate that FGFR5 administration to mice induces B cell expansion and hyperactivation suggesting that the level of serum immunoglobulin (Ig) may be elevated in these animals. In order to examine this, serum samples collected from the animals intraperitoneally treated with FGFR5β or FGFR2 for 3 weeks (as described in Example 22) were analyzed for murine Ig using an ELISA assay. Sera from 8.5 month old NZB/W F1 mice with an SLE-like disease was used as a positive control and sera from untreated BALB/c mice as a negative control.

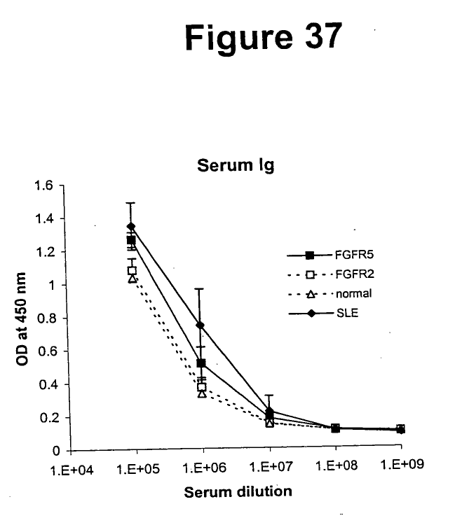

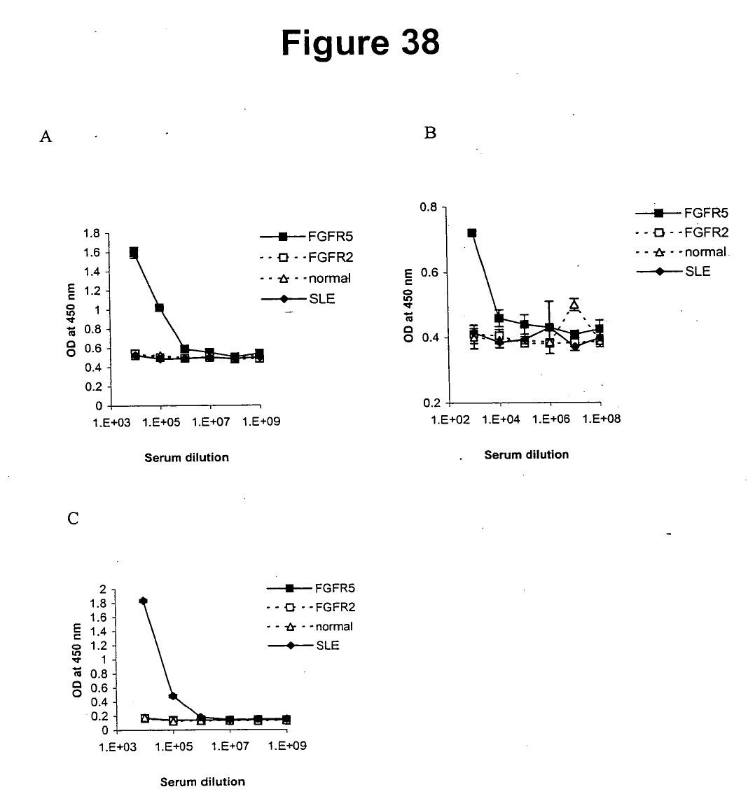

Briefly, goat anti-total mouse Ig was coated onto 96-well ELISA plates (Nunc Immuno-Plate), then serial dilutions of sera were incubated for 2 hrs at room temperature and the bound mouse Ig was detected using horseradish peroxidase (HRP)-conjugated goat anti-total mouse Ig. As shown in FIG. 37, the sera from FGFR5β-treated mice contained more Ig than either the FGFR2-treated or untreated mice, although the levels were lower than in NZB/W F1 mice. Upon further analysis, IgG1 (FIG. 38A) and IgE (FIG. 38B) levels were found to be significantly increased in the FGFR5β-treated group while high titres of IgG2a (FIG. 38C) were noted in NZB/W F1 mice. Levels of IgM and IgA in sera were not significantly different among all groups of mice (data not shown).

The ability of FGFR5 to induce high levels of osteopontin secretion, its chromosomal location and ability to activate B cells indicates that it may play in autoimmune diseases such as SLE. One of the hallmarks of SLE in mice and man is the presence of elevated levels of anti-double stranded DNA (anti-dsDNA). To determine whether FGFR5 induced an SLE-like autoimmune disease in mice the sera from FGFR5β- and FGFR2-treated BALB/c mice were examined for presence of anti-dsDNA by ELISA as follows.

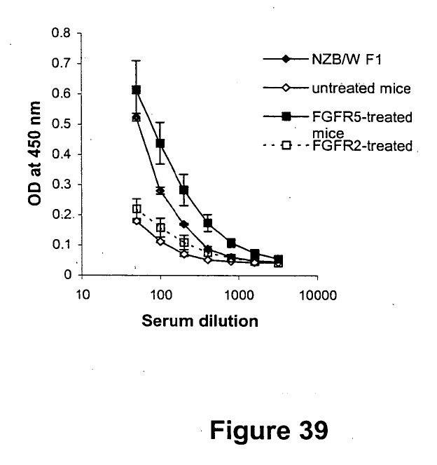

Microtiter plates were incubated with native calf thymus dsDNA (Sigma). Serial dilutions of sera were incubated and bound Ig was detected with HRP-conjugated goat anti-mouse Ig. A serum pool of 8.5 month old NZB/W F1 mice was used as an internal positive control in all assays. High titers of autoantibody were detected in the sera of FGFR5β-treated mice, which were even higher than those observed in NZB/W F1 mice (FIG. 39). In contrast, the presence of such autoantibodies was undetectable in the sera of FGFR2-treated mice and untreated littermates (only background absorbance was obtained in sera of these mice). The Ig subclass anti-dsDNA activity was further evaluated and showed that in FGFR5β-treated mice, anti-dsDNA antibodies were predominantly of the IgG1 and IgE, whereas IgG2a anti-dsDNA was present in the sera of NZB/W F1 mice (data not shown). The induction of hyperglobulinemia and autoantibody by FGFR5 demonstrated in this study provides compelling evidence that FGFR5 is associated with the development of autoimmune diseases and that its antagonists may have therapeutic potential for these diseases.

In addition to polyclonal B cell activation and autoantibody production, the presence of high serum Ig levels in FGFR5β-treated mice may also be associated with the humoral immunity against the recombinant proteins injected. To verify whether or not administration of FGFR5β can induce an antigen-specific response, we analyzed the sera of murine FGFR5β-human Fc fusion protein-treated or human FGFR2-human Fc fusion protein-treated mice for antibodies against the recombinant protein and the human Fc using an ELISA assay.

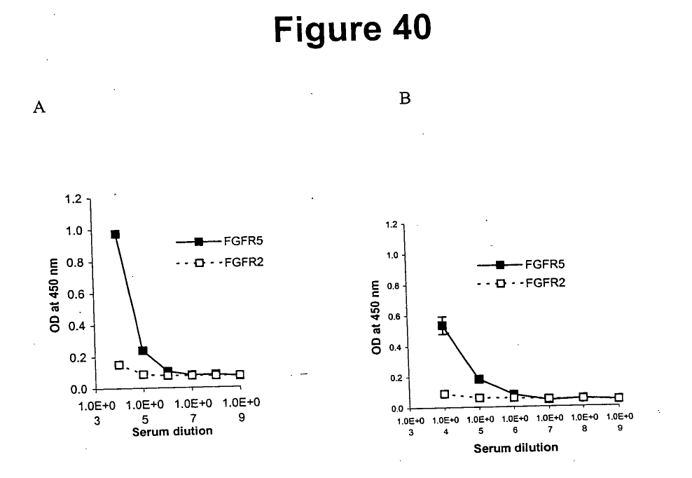

ELISA plates were coated with human IgG or murine monomeric FGFR5β that was cleaved from FGFR5-Fc fusion protein and contained traces of human Fc, then incubated with serial dilutions of sera. Serum antibody levels were detected using HRP-conjugated goat anti-mouse Ig. Much higher levels of anti-human Fc antibody were detected in sera of FGFR5β-treated mice than in FGFR2-administered animals (FIG. 40A). Both groups of mice were administered recombinant proteins containing the same human Fc portion but had different magnitudes of immune response. This difference may be explained by the fact that FGFR5, a B cell stimulator, induced B cell hyperactivity that resulted in a higher response of B cells against the human Fc portion. These observations indicate that FGFR5 has an immune regulatory effect and may be employed as an adjuvant for use in the treatment of infectious diseases and cancers.

Modest levels of antibodies were noted in the wells incubated with monomeric FGFR5β0 and sera obtained from FGFR5-treated mice (FIG. 40B), indicating the possible presence of anti-FGFR5β antibodies. However, it is also possible that these antibodies are anti-human Fc that bound to the contaminating human Fc in the monomeric FGFR5β.

### EXAMPLE 21

### FGFR5 Induces Osteoclast Formulation In Vivo

The mouse bone marrow assay was used to assess the effects of FGFR5 on osteoclast development as follows.

A single cell suspension of mouse bone marrow cells was obtained from femurs by flushing them with cold IMDM supplemented with 2% FBS, pipetting gently and passing through 70-μm nylon filter. Red blood cells were removed using a hypotonic ammonium chloride lysis buffer. Cells were then washed, suspended in RPMI 1640 containing 10% heat-inactivated fetal calf serum, and added to 6 mm diameter chamber slides (Nunc, Denmark) at 5×10⁵ cells/slide. The cells were stimulated with recombinant murine FGFR5β human Fc fusion protein at 5, 25 or 100 nM, or recombinant human FGFR2 human Fc protein at the same concentrations as a control for the Fc fragment of the protein. In addition, separate wells were also cultured with either media or the osteoclastogenic combination of recombinant soluble RANKL (50 ng/ml) (R&D Systems, Minn., USA) and recombinant M-CSF (50 ng/ml) (R&D Systems, Minn., USA). In all cultures, medium (with added factors) was entirely replaced every three days. After 7 days, the cells were fixed and stained for tartrate-resistant acid phosphatase (TRAP) using a commercial kit (Sigma). Photomicrographs (400×) were taken of 10 different fields of each culture and the numbers of osteoclasts per field determined by counting the TRAP-positive multinucleated cells containing greater than three nuclei.

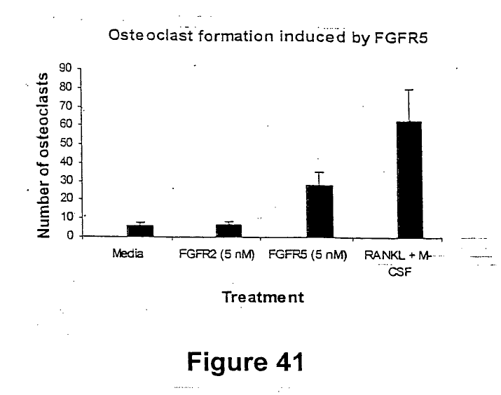

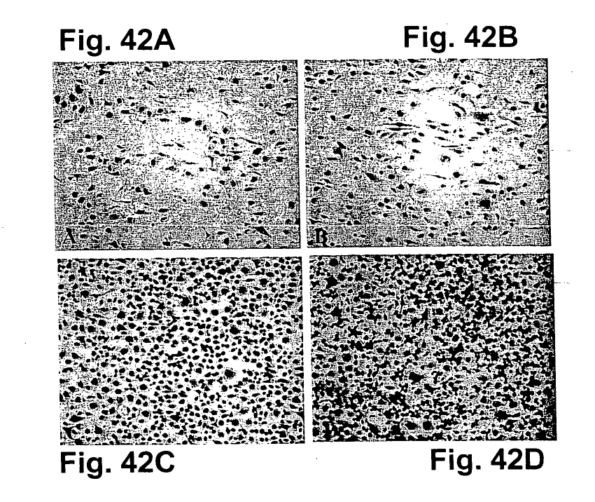

FIGS. 41 and 42A-D show that few TRAP-positive multinucleated osteoclasts formed in untreated cultures (media control) and that the addition of FGFR2 at all concentrations tested had no effect on the formation of osteoclasts. However, FGFR5β significantly increased the number of osteoclasts (Student t test, p<0.0001) as compared to FGFR2 and media control. Similar effects were seen in all of the FGFR5β-stimulated cultures irrespective of the concentration used. The number of osteoclasts from cultures treated with 5 nM proteins are shown in FIG. 41, in which the values are the mean±SD for two independent experiments. FIG. 42A-D provides photomicrographs demonstrating the effect on FGFR5 on osteoclast formation. FGFR5β was also found to have the capacity to induce osteoclastogenesis from human monocytes in vitro (data not shown).

### EXAMPLE 22

### Expression of FGFR5 in Zebrafish

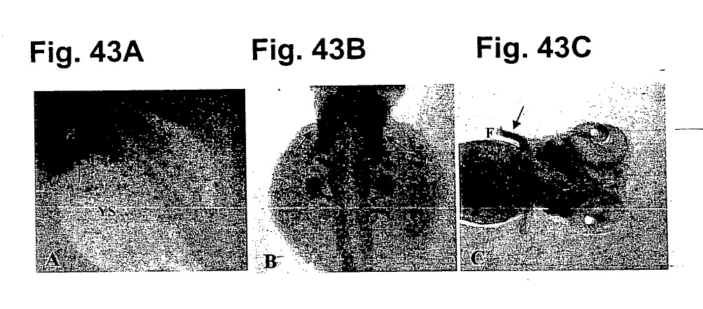

Gene expression of FGFR5 in Zebrafish embryos was determined at the Developmental Genetics and Leukemia Laboratory, Faculty of Medical and Health Sciences, University of Auckland, by in situ hybridization using a riboprobe constructed from the full-length zebrafish FGFR5 (accession number BC053245) and a control probe. FIG. 43A shows that FGFR5 mRNA was readily detected on the head of embryo 24 hours post fertilization (hpf). At 48 hpf, strong expression of FGFR5 was observed in the developing fins and other bony structures (FIG. 43B). The expression of FGFR5 in bone was clearly evident 5 days post fertilization (dpf) with FGFR5 expression in the pharyngeal arches and fins (FIG. 43C). There was no evidence of hybrization with the control probe (data not shown).

Zebrafish (Danio rerio) FGFR5-knockdowns were generated using morpholino oligonucleotides as described below. Morpholino oligonucleotides were designed to interfere with correct splicing of the second Ig domain, resulting in a transcript devoid of this region. FGFR5 morpholinos (FGFR5mo) were injected at 2 different doses into the yolk cell of one- to four-cell embryos just beneath the animal's cell(s). At a dose of 1 pmol (about 8 ng) per injection, about 50%-65% of the injected Zebrafish generated the phenotype described below, and at 1.5 pmol (about 12 ng) per injection about 100% of the zebrafish generated the phenotype (out of 100-150 tested zebrafish). FGFR5mo-injected zebrafish were compared with uninjected wildtype animals.

Whole mount in situ hybridization analysis of fgfr5 expression during zebrafish embrogenesis revealed the presence of fgfr5 transcripts initially in the developing somites commencing from mid-somitogenesis. This expression domain persisted throughout early development, although becoming confined more to the somite boundaries at later stages. Additional domains included the otic vesicle, developing head, pectoral fins, perianal region, heart and presumptive pharyngeal endoderm.

At the 8-somite stage (about 13 hours post fertilization, hpf) when the expression of fgfr5 is first detected in the somites, the FGFR5 morphants (Zebrafish injected with FGFR5mo) did not show gross morphological abnormalities when compared with uninjected siblings. However, at 24 hpf the somites of the morphants appeared denser and irregular in shape. This is possibly due to a patterning defect, as major elements in somite formation are normal, i.e. segmentation; myosepta, etc. Defects in the head region became obvious at this stage of development. The brain region looked smaller and the eyes underdeveloped. These defects were more apparent at 30 hpf when the hindbrain ventricle appeared enlarged. In more severe cases, although distinction between anterior and posterior regions of the brain, as well as the midbrain-hindbrain boundary was intact, morphological development appeared to be delayed.

At 48 hpf, the enlarged hindbrain was still clearly visible, and the epithelium projections in the inner ear that form the semicircular canals were absent in a subset of morphants. The eyes were markedly smaller with reduced pigmentation in the ventral half.

At 5 days post fertilization (dpf), FGFR5 morphants also showed a great significant defect in the development of the pharyngeal arches (PA). Whereas in the wildtype the cartilage component of all seven pharyngeal arches had clearly developed at this stage, PA 3-6 were absent, and only remnants of PA 7 could be visualized by alcian blue staining in the FGFR5-depleted background. The mandibular (PA 1) and hyoid (PA 2) arch cartilage were present but dysmorphic. Alcian blue specifically stains proteoglycan in cartilaginous tissue. In addition, a heart defect was apparent, resulting in slower blood flow (inefficient pumping of the blood). This labored circulatory defect results in substantial pericardial oedema and is most likely the reason for death of the FGFR5 morphants by day 7 (zebrafish embryos can survive for the first week of development in the absence of blood flow due to passive diffusion of oxygen into tissues). Preliminary analysis of this defect suggests that FGFR5 is not involved in formation of the heart as a linear 2-chambered contracting heart tube does form, but may be involved in the subsequent modelling of this heart tube.

Overall, the knockdown of FGFR5 in zebrafish at an early developmental stage resulted in severe defects of the head (brain, eye, ear), the heart and the trunk, and resulted in the death of the fish by day 7. It is interesting to note that FGFR5 appeared to be key to the development of some but not all bones in the zebrafish which is consistent with the studies by Trueb et al (Jnl. Biol. Chem., 278:33857-33865, 2003) showing that it is expressed in the bones of mice and chickens, and studies described above indicating that it plays a role in osteoclastogenesis. These results support the role of FGFR5 in bone development and indicate that FGFR5 or FGFR5 antagonists may be of therapeutic benefit in bone diseases such as osteoporosis.

The phenotype of the FGFR5 morphants was very similar to the FGF3-knockdown and the FGF3 null mutant lia in zebrafish. FGF3 is thought to be the ligand for FGFR1, however the FGFR1 knockdown does not result in the same phenotype as the FGF3 knockdown. Thus FGF3 may be a ligand for FGFR5.

Methods

Zebrafish Stocks:

Zebrafish (Danio rerio) embryos were obtained from natural spawning between wild-type (AB, Oregon stock centre) adult fish. Embryos were raised at 28° C. in Embryo Medium (Westerfield, M. (2000). The zebrafish book. A guide for the laboratory use of zebrafish (Danio rerio). 4th ed., Univ. of Oregon Press, Eugene) and staged according to Kimmel et al. (Dev. Dyn. 203: 253-310, 1995).

Antisense Probe Synthesis:

To construct a cDNA template encoding zebrafish Fgfr5, total RNA was extracted from approximately one hundred 24 hours post fertilisation (hpf) zebrafish embryos using Trizol (Life Technologies). From this, first strand cDNA was synthesised using 2 μg total RNA, 500 ng oligo dT primers (Roche), 10 mM DTT, 500 μM dNTPs and 200 units Superscript II Reverse Transcriptase (Invitrogen). The entire coding region of Fgfr5 was then amplified using oligonucleotides Fgfr5 oligo A, 5′ GAGGAACAGATTTCTGATCATACTTTC 3′ (SEQ ID NO: 155) and Fgfr5 oligo F 5′ CATTTGTTTGTTACCCTTGCCC 3′ (SEQ ID NO: 156). A 1.7 kb PCR product was generated using the following cycling conditions: 3 minutes at 94° C., 35 cycles of 94° C. for 45 seconds, 51° C. for 30 seconds and 72° C. for 1.5 minutes followed by 10 minutes at 72° C. This PCR product was then cloned into the pGEM-T Easy Vector (Promega) for probe synthesis and sequencing verification. RNA probes were synthesized using the DIG RNA labeling kit (Roche) with the following combinations: ApaI linearized template and SP6 for antisense, SpeI linearized template and T7 for sense.

Expression Analysis:

Whole-mount in situ hybridizations were performed essentially as described (Jowet, T., Lettice, L., Trends Genetic. 10:73-74, 1994), except that the hybridization temperature was optimized at 65° C. and BM purple (Boehringer-Mannheim) was used for staining. Pigmentation in embryos older than 24 hpf was inhibited by raising embryos in E3 medium supplemented with PTU (I-phenyl-2-thiourea; Sigma) as described in Westerfield, 2000 (Ibid). Alternatively, pigmentation was removed in post-fixation embryos by bleaching in a solution containing 5% formamide, 0.5×SSC and 10% H₂O₂.

Injection of Morpholino Oligonucleotides:

Morpholinos (Gene Tools, LLC), received as sterile salt-free lyophilized solids, were resuspended in sterile water to a concentration of 50 mg/ml. For injection, this stock solution was diluted to 3 mg/ml with 1×Danieau [58 mM NaCl, 0.7 mM KCl, 0.4 mM MgSO₄, 0.6 mM Ca(NO₃)₂, 0.5 mM Hepes, pH 7.6] and typically injected at a volume of 2 to 3 nl at the yolk/cytoplasm interface. Immediately following injection, embryos were left to recover at 28° C. in Embryo Medium. A splice blocking Morpholino oligo (Fgfr5SB1, 5′ TGTGTGACTCACGGATGACTTCCAC 3′ (SEQ ID NO: 157), sequence highlighted in bold and italics represents sequence complementary to intronic and exonic sequences, respectively) was designed to specifically bind to and interfere with the splice donor site immediately downstream of the exon encoding the second Ig domain of Fgfr5.

RT-PCR:

RT-PCR was employed to verify successful interference with normal splicing of Fgfr5 pre-mRNA. Total RNA was isolated from approximately 50 to 75 wild-type and Fgfr5SB1-injected embryos following 30 hours development using Trizol (Life Technologies). First stand cDNA was then generated using Superscript II reverse transcriptase (Invitrogen) and oligo dT primers (Roche), reverse transcriptase negative controls (reactions lacking Superscript II RT) were also employed. PCR was performed using 1 μl cDNA and the following oligonucleotides pairs designed to specifically amplify defined regions of the fgfr5 transcript: Fgfr5 oligo A, 5′ GAGGAACAGATTTCTGATCATACTTTC 3′ (SEQ ID NO: 155) and Fgfr5 oligo B, 5′ CCTCTTTGATGCGGAGAGCTTGC 3′ (SEQ ID NO: 158) amplify a 317 bp product; Fgfr5 oligo C, 5′ CAATATCAACTACACCCTCATCG 3′ (SEQ ID NO: 159) and Fgfr5 oligo E, 5′ CTTGACATCACTGCGTACTTTGC 3′ (SEQ ID NO: 160) amplify a 498 bp product; Fgfr5 oligo D, 5′ CAAAATGAGAAAGCGTGTGATTGC 3′ (SEQ ID NO: 161) and Fgfr5 oligo E amplify a 366 bp product; Fgfr5 oligo A and Fgfr5 oligo F amplify a 1,668 bp product (predicted product sizes based on wild-type transcript). Oligonucleotides designed to amplify a 385 bp fragment of the wnt5a transcript were also used as a positive control, Wnt5a antisense, 5′ CTTCCGGCGTGTTGGAGAATTC 3′ (SEQ ID NO: 162) and Wnt5a sense 5′ CAGTTCTCACGTCTGCTACTTGCA 3′ (SEQ ID NO: 163). Cycling conditions for PCR reactions involving oligo pairs A/B, C/E , D/E and Wnt5a sense/antisense were: 3 minutes at 94° C., 30 cycles of 94° C. for 30 seconds, 55° C. for 30 seconds and 72° C. for 30 seconds followed by 10 minutes at 72° C., while those involving A/F were as described above (see Antisense probe synthesis).

Alcian Blue Staining:

To visualize craniofacial cartilage, 5 dpf embryos were stained with Alcian blue (Sigma) as described (Schilling et al., Development 122:1417-1426, 1996).

Sectioning:

Stained embryos to be sectioned were dehydrated in methanol and embedded in JB-4 methacrylate (Polysciences). Sections 5 to 8 μm thick were cut using a RM2155 microtome (Leica) and transferred to glass slides, counterstained with Nuclear Fast Red (Vector Laboratories, Inc.) and mounted with Poly-Mount (Polysciences).

Imaging:

Images were captured using a Leica DC200 digital camera connected to a Leica MZFLIII stereo microscope. Video images of the cardiovascular system were obtained using a Nikon Coolpix 4500 digital camera mounted to a Leica MZFLIII stereo microscope.

Although the present invention has been described in terms of specific embodiments, changes and modifications can be carried out without departing from the scope of the invention which is intended to be limited only by the scope of the appended claims. All references cited herein, including patent references and non-patent references, are hereby incorporated by reference in their entireties.

SEQ ID NO: 1-163 are set out in the attached Sequence Listing. The codes for polynucleotide and polypeptide sequences used in the attached Sequence Listing conform to WIPO Standard ST.25 (1988), Appendix 2.

1. An isolated polypeptide comprising a sequence selected from the group consisting of: SEQ ID NO: 5-8 and 13-15.

2. An isolated polypeptide comprising a sequence selected from the group consisting of:
(a) sequences having at least 75% identity to a sequence provided in SEQ ID NO: 5-8 and 13-15;
(b) sequences having at least 90% identity to a sequence provided in SEQ ID NO: 5-8 and 13-15; and
(c) sequences having at least 95% identity to a sequence provided in SEQ ID NO: 5-8 and 13-15,
wherein the polypeptide possesses at least one functional property that is substantially the same as a functional property of a sequence of SEQ ID NO: 5-8 and 13-15.

3. An isolated polynucleotide that encodes a polypeptide according to any one of claims 1 and 2.

4. An isolated polynucleotide of claim 3, wherein the polynucleotide comprises a sequence selected from the group consisting of: sequences provided in SEQ ID NO: 1-4 and 9.

5. An isolated polynucleotide comprising a sequence selected from the group consisting of:
(a) complements of a sequence provided in SEQ ID NO: 1-4 and 9;
(b) reverse complements of a sequence provided in SEQ ID NO: 1-4 and 9;
(c) reverse sequences of a sequence provided in SEQ ID NO: 1-4 and 9;
(d) sequences having at least 75% identity to a sequence provided in SEQ ID NO: 1-4 and 9; and
(e) sequences having at least 90% identity to a sequence provided in SEQ ID NO: 1-4 and 9; and
(f) sequences having at least 95% identity to a sequence of SEQ ID NO: 1-4 and 9.

6. An isolated polynucleotide comprising a sequence selected from the group consisting of: (a) sequences that are a 200-mer of an isolated polynucleotide according to any one of claims 3, 4 and 5; (b) sequences that are a 100-mer of an isolated polynucleotide according to any one of claims 3, 4 and 5; and (c) sequences that are a 40-mer of an isolated polynucleotide according to any one of claims 3, 4 and 5.

7. An expression vector comprising an isolated polynucleotide according to any one of claims 3-6.

8. A host cell transformed with an expression vector according to claim 7.

9. An isolated polypeptide comprising at least a functional portion of an amino acid sequence selected from the group consisting of sequences provided in SEQ ID NO: 5-8 and 13-15.

10. A fusion protein comprising at least one polypeptide according to any one of claims 1, 2 and 9.

11. A composition comprising an isolated polypeptide according to any one of claims 1, 2 and 9 and at least one component selected from the group consisting of: physiologically acceptable carriers and immunostimulants.

12. A composition comprising an isolated polynucleotide according to any one of claims 3-6 and at least one component selected from the group consisting of: physiologically acceptable carriers and immunostimulants.

13. A composition comprising a fusion protein according to claim 10 and at least one component selected from the group consisting of: physiologically acceptable carriers and immunostimulants.

14. A composition comprising a modulator of FGFR5 gene expression, wherein said modulator is selected from the group consisting of: (a) small molecule inhibitors of FGFR5 gene expression; (b) anti-sense oligonucleotides to FGFR5; and (c) small interfering RNA molecules (siRNA).

15. The composition of claim 14, wherein said modulator of FGFR5 gene expression is able to modulate osteopontin expression in a population of cells.

16. The composition of claim 15, wherein said modulator of FGFR5 gene expression specifically binds to a polynucleotide of claim 3.

17. The composition of claim 14 wherein said modulator of FGFR5 gene expression is an anti-sense oligonucleotide, and wherein said anti-sense oligonucleotide is selected from the group consisting of: (a) anti-sense expression vectors; (b) anti-sense oligodeoxyribonucleotides; (c) anti-sense phosphorothioate oligodeoxyribonucleotides; (d) anti-sense oligoribonucleotides; and (e) anti-sense phosphorothioate oligoribonucleotides.

18. A composition comprising a binding agent that specifically binds to an FGFR5 polypeptide and is able to modulate osteopontin expression in a population of cells, wherein said binding agent is selected from the group consisting of: (a) small molecules; (b) antibodies or antigen-binding fragments thereof; (c) small chain antibody fragments (scFv); (d) camelid heavy chain antibodies (HCAb) or heavy chain variable domains thereof (V_{HH}); and (e) FGFR5 ligands or antigen-binding fragments thereof.

19. The composition of claim 18, wherein the binding agent specifically binds to a polypeptide of any one of claims 1 and 2.

20. The composition of claim 18, wherein the binding agent is an antagonist of FGFR5 polypeptide function.

21. A method for the treatment of a disorder of the immune system in patient, comprising administering to the patient a composition according to any one of claims 11-13.

22. A method for the treatment of cancer in a patient, comprising administering to the patient a composition according to any one of claims 11-13, wherein the cancer is selected from the group consisting of epithelial, lymphoid, myeloid, stromal and neuronal cancers.

23. A method for the treatment of a viral disorder in a patient, comprising administering to the patient a composition according to any one of claims 11-13.

24. The method of claim 23, wherein the viral disorder is HIV-infection.

25. A method for the treatment of a fibroblast growth factor-mediated disorder in a patient, comprising administering a composition according to any one of claims 11-13.

26. A method for modulating an immune response in a patient, comprising administering to the patient a composition according to any one of claims 11-13.

27. A method for inhibiting the expression of osteopontin in a population of cells, comprising reducing the amount of a polypeptide in the cells, the polypeptide comprising an amino acid sequence selected from the group consisting of:
(a) a sequence provided in SEQ ID NO: 5-8 and 13-15;
(b) sequences having at least 75% identity to a sequence provided in SEQ ID NO: 5-8 and 13-15
(c) sequences having at least 90% identity to a sequence provided in SEQ ID NO: 5-8 and 13-15; and
(d) sequences having at least 95% identity to a sequence provided in SEQ ID NO: 5-8 and 13-15.

28. A method for inhibiting the expression of osteopontin in a population of cells, comprising inhibiting the activity of a polypeptide in the cells, the polypeptide comprising an amino acid sequence selected from the group consisting of:
(a) a sequence provided in SEQ ID NO: 5-8 and 13-15;
(b) sequences having at least 75% identity to a sequence provided in SEQ ID NO: 5-8 and 13-15;
(c) sequences having at least 90% identity to a sequence provided in SEQ ID NO: 5-8 and 13-15; and
(d) sequences having at least 95% identity to a sequence provided in SEQ ID NO: 5-8 and 13-15.

29. The method of claim 28, wherein the method comprises contacting the cells with an antibody, or an antigen-binding fragment thereof that binds specifically to a polypeptide comprising an amino acid sequence selected from the group consisting of:
(a) sequences provided in SEQ ID NO: 5-8 and 13-15;
(b) sequences having at least 75% identity to a sequence provided in SEQ ID NO: 5-8 and 13-15;
(c) sequences having at least 90% identity to a sequence provided in SEQ ID NO: 5-8 and 13-15; and
(d) sequences having at least 95% identity to a sequence provided in SEQ ID NO: 5-8 and 13-15.

30. The method of claim 28, wherein the method comprises contacting the cells with an anti-sense oligonucleotide that binds specifically to a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

31. The method of claim 28, wherein the method comprises contacting the cells with a small interfering RNA molecule that suppresses expression of a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

32. A method for treating a disorder characterized by an elevated level of osteopontin, comprising administering an antibody, or an antigen-binding fragment thereof that binds specifically to a polypeptide comprising an amino acid sequence selected from the group consisting of:
(a) sequences provided in SEQ ID NO: 5-8 and 13-15;
(b) sequences having at least 75% identity to a sequence provided in SEQ ID NO: 5-8 and 13-15;
(c) sequences having at least 90% identity to a sequence provided in SEQ ID NO: 5-8 and 13-15; and
(d) sequences having at least 95% identity to a sequence provided in SEQ ID NO: 5-8 and 13-15.

33. A method for treating a disorder characterized by an elevated level of osteopontin, comprising administering an anti-sense oligonucleotide that binds specifically to a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

34. A method for treating a disorder characterized by an elevated level of osteopontin, comprising administering a small interfering RNA molecule that corresponds to a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

35. The method of any one of claims 32-34, wherein the disorder is selected from the group consisting of: multiple sclerosis; systemic lupus erythematosus; diabetes; rheumatoid arthritis; sarcoidosis; tuberculosis; kidney stones; atherosclerosis; vasculitis; nephritis; arthritis; and osteoporosis.


SEQUENCE LISTING (163 sequences)

SEQ ID NO: 1 - DNA, 384 bp, Mouse
  misc_feature (1)...(384): n = A,T,C or G
ggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggngatcggcatcccagc

SEQ ID NO: 2 - DNA, 1967 bp, Mouse
gctgcgcgcccccgcgctgatccctgtcgagcgtctacgcgcctcgcttcctttgcctggagctcggcgccgaggggggccggaccctggctctgcggccgcgacctgggtcttgcgggcctgagccctgagtggcgtccagtccagctcccagtgaccgcgcccctgcttcaggtccgaccggcgagatgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatatgggggccaaggcaacaggttgggagaattgagaacaatggaggaagagtatcttagggtgccttatggtggacactcacaaacttggccatatagatgtatgtactaccagatgaacagccagccagattcacacacgcacatgtttaaacgtgtaaacgtgtgcacaactgcacacacaacctgagaaaccttcaggaggatttggggtgtgactttgcagtgacatgtagcgatggctagttg

SEQ ID NO: 3 - DNA, 1742 bp, Mouse
gcgcggcgccccgggcccctcgccccgccgcccctcttccccgccctcgccaagcctcgccgtttatccgcgcggacagcgcgccccgcgccccagcccggccctagccgccagcgcccaggtagcgccgccccgcccaggccgggcccgggggcgcggggggcgggatgcggcgcccggggcagcgatgaccgcgtcgcgctgctcaggggcccggctctgaccccgttgcctgctgcgcgcccccgcgctgatccctgtcgagcgtctacgcgcctcgcttcctttgcctggagctcggcgccgaggggggccggaccctggctctgcggccgcgacctgggtcttgcgggcctgagccctgagtggcgtccagtccagctcccagtgaccgcgcccctgcttcaggtccgaccggcgagatgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctcctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatatgggggccaaggcaacaggttgggagaattgagaacaatggaggaagagtatcttag

SEQ ID NO: 4 - DNA, 1006 bp, Human
  misc_feature (1)...(1006): n = A,T,C or G
gcggccgcgaccccaggtccggacaggccgagatgacgccgagccccctgttgctgctcctgctgccgccgctgctgctgggggccttcccaccggccgccgccgcccgaggccccccaaagatggcggacaaggtggtcccacggcaggtggccnggctgggccgcactgtgcggctgcagtgccnagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagccgctccc

SEQ ID NO: 5 - protein, 126 aa, Mouse
  VARIANT (1)...(126): Xaa = Any Amino Acid
VDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVXGIP

SEQ ID NO: 6 - protein, 529 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 7 - protein, 439 aa, Mouse
MTRSPALLLLLLGALPSAEAARDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 8 - protein, 324 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKPL

SEQ ID NO: 9 - DNA, 1423 bp, Mouse
tctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccgtgagtggtgggtctgtggtaggacaggggcccgtggtgcctaaaactgtgctgacatgtttgtttttccttggcttagagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccaggtgtgtgtgtgggctgcccaccccatgtttactctcagtctctaccattggtctgggctgtcctggggttccccaatgtccacttagcaagtggggcctccctatccttttcccttcgttgtgggttatccttgcctcatagggagttcaggggtgctgcccatatagttcacatttgggctggttgccccattaatatagggacattctgtcccctactcttcttcttaatctctcttgcagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatatgggggccaaggcaacaggttgggagaattgagaacaatggaggaagagtatct

SEQ ID NO: 10 - DNA, 35 bp, Artificial Sequence
  Made in a lab
cgggatccaggccatggcaggcttgtggatgacga

SEQ ID NO: 11 - DNA, 37 bp, Artificial Sequence
  Made in a lab
ccgctcgagtagatactcttcctccattgttctcatt

SEQ ID NO: 12 - DNA, 18 bp, Artificial Sequence
  Made in a lab
ctgtgcggctcaagtgtg

SEQ ID NO: 13 - protein, 373 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPW

SEQ ID NO: 14 - protein, 135 aa, Mouse
CQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 15 - protein, 37 aa, Mouse
RVEYGSEGRHNSTIDVGGQKFVVLPTGDVWSRPDGSY

SEQ ID NO: 16 - DNA, 1515 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 17 - protein, 504 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 18 - DNA, 1161 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 19 - protein, 386 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIHPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 20 - DNA, 1230 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 21 - protein, 409 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 22 - DNA, 1434 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 23 - protein, 477 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 24 - DNA, 1242 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 25 - protein, 413 aa, Human
MTPSPLLLLLLPPLLLGAFPPAAAARDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 26 - DNA, 876 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 27 - protein, 291 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 28 - DNA, 1080 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 29 - protein, 359 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLARPRFTQPSKMRRRVIARPVGSSVRLKCVASGHPRPDITWMKDDQALTRPEAAEPRKKKWTLSLKNLRPEDSGKYTCRVSNRAGAINATYKVDVIHPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 30 - DNA, 1149 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 31 - protein, 382 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 32 - DNA, 888 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 33 - protein, 295 aa, Human
MTPSPLLLLLLPPLLLGAFPPAAAARDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIHPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 34 - DNA, 957 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtgggagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 35 - protein, 318 aa, Human
MTPSPLLLLLLPPLLLGAFPPAAAARDDISPGKESLGPDSSSGGQEDPASQQWERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 36 - DNA, 1161 bp, Human
atgacgccgagccccctgttgctgctcctgctgccgccgctgctgctgggggccttcccaccggccgccgccgcccgagcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 37 - protein, 386 aa, Human
MTPSPLLLLLLPPLLLGAFPPAAAARARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 38 - DNA, 795 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 39 - protein, 264 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 40 - DNA, 603 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtgggacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 41 - protein, 200 aa, Human
MTPSPLLLLLLPPLLLGAFPPAAAARDDISPGKESLGPDSSSGGQEDPASQQWDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 42 - DNA, 807 bp, Human
atgacgccgagccccctgttgctgctcctgctgccgccgctgctgctgggggccttcccaccggccgccgccgcccgagcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 43 - protein, 268 aa, Human
MTPSPLLLLLLPPLLLGAFPPAAAARARPRFTQPSKMRRRVIARPVGSSVRLKCVASGHPRPDITWMKDDQALTRPEAAEPRKKKWTLSLKNLRPEDSGKYTCRVSNRAGAINATYKVDVIHPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 44 - DNA, 876 bp, Human
atgacgccgagccccctgttgctgctcctgctgccgccgctgctgctgggggccttcccaccggccgccgccgcccgagagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 45 - protein, 291 aa, Human
MTPSPLLLLLLPPLLLGAFPPAAAARERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 46 - DNA, 522 bp, Human
atgacgccgagccccctgttgctgctcctgctgccgccgctgctgctgggggccttcccaccggccgccgccgcccgagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctag

SEQ ID NO: 47 - protein, 173 aa, Human
MTPSPLLLLLLPPLLLGAFPPAAAARDPKPQGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC

SEQ ID NO: 48 - DNA, 1072 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 49 - protein, 357 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 50 - DNA, 718 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatcc

SEQ ID NO: 51 - protein, 239 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVI

SEQ ID NO: 52 - DNA, 787 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 53 - protein, 262 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 54 - DNA, 991 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 55 - protein, 330 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 56 - DNA, 799 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 57 - protein, 266 aa, Human
MTPSPLLLLLLPPLLLGAFPPAAAARDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 58 - DNA, 433 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtggg

SEQ ID NO: 59 - protein, 144 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQW

SEQ ID NO: 60 - DNA, 637 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatcc

SEQ ID NO: 61 - protein, 212 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLARPRFTQPSKMRRRVIARPVGSSVRLKCVASGHPRPDITWMKDDQALTRPEAAEPRKKKWTLSLKNLRPEDSGKYTCRVSNRAGAINATYKVDVI

SEQ ID NO: 62 - DNA, 706 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 63 - protein, 235 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 64 - DNA, 445 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatcc

SEQ ID NO: 65 - protein, 148 aa, Human
MTPSPLLLLLLPPLLLGAFPPAAAARDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVI

SEQ ID NO: 66 - DNA, 514 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtgggagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 67 - protein, 171 aa, Human
MTPSPLLLLLLPPLLLGAFPPAAAARDDISPGKESLGPDSSSGGQEDPASQQWERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 68 - DNA, 718 bp, Human
atgacgccgagccccctgttgctgctcctgctgccgccgctgctgctgggggccttcccaccggccgccgccgcccgagcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 69 - protein, 239 aa, Human
MTPSPLLLLLLPPLLLGAFPPAAAARARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 70 - DNA, 352 bp, Human
atgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctgg

SEQ ID NO: 71 - protein, 117 aa, Human
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVL

SEQ ID NO: 72 - DNA, 160 bp, Human
atgacgccgagccccctgttgctgctcctgctgccgccgctgctgctgggggccttcccaccggccgccgccgcccgagatgacattagcccagggaaggagagcctggggcccgacagctcctctgggggtcaagaggaccccgccagccagcagtggg

SEQ ID NO: 73 - protein, 53 aa, Human
MTPSPLLLLLLPPLLLGAFPPAAAARDDISPGKESLGPDSSSGGQEDPASQQW

SEQ ID NO: 74 - DNA, 364 bp, Human
atgacgccgagccccctgttgctgctcctgctgccgccgctgctgctgggggccttcccaccggccgccgccgcccgagcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatcc

SEQ ID NO: 75 - protein, 121 aa, Human
MTPSPLLLLLLPPLLLGAFPPAAAARARPRFTQPSKMRRRVIARPVGSSVRLKCVASGHPRPDITWMKDDQALTRPEAAEPRKKKWTLSLKNLRPEDSGKYTCRVSNRAGAINATYKVDVI

SEQ ID NO: 76 - DNA, 433 bp, Human
atgacgccgagccccctgttgctgctcctgctgccgccgctgctgctgggggccttcccaccggccgccgccgcccgagagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccag

SEQ ID NO: 77 - protein, 144 aa, Human
MTPSPLLLLLLPPLLLGAFPPAAAARERTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLP

SEQ ID NO: 78 - DNA, 79 bp, Human
atgacgccgagccccctgttgctgctcctgctgccgccgctgctgctgggggccttcccaccggccgccgccgcccgag

SEQ ID NO: 79 - protein, 26 aa, Human
MTPSPLLLLLLPPLLLGAFPPAAAAR

SEQ ID NO: 80 - DNA, 1590 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 81 - protein, 529 aa, Mouse; identical to SEQ ID NO: 6

SEQ ID NO: 82 - DNA, 1236 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 83 - protein, 411 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIHPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 84 - DNA, 1305 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 85 - protein, 434 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 86 - DNA, 1509 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 87 - protein, 502 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 88 - DNA, 1317 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 89 - protein, 438 aa, Mouse
MTRSPALLLLLLGALPSAEAARDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 90 - DNA, 951 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 91 - protein, 316 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 92 - DNA, 1155 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 93 - protein, 384 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMARPRFTQPSKMRRRVIARPVGSSVRLKCVASGHPRPDIMWMKDDQTLTHLEASEHRKKKWTLSLKNLKPEDSGKYTCRVSNKAGAINATYKVDVIHPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 94 - DNA, 1224 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 95 - protein, 407 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 96 - DNA, 963 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 97 - protein, 320 aa, Mouse
MTRSPALLLLLLGALPSAEAARDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIHPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 98 - DNA, 1032 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 99 - protein, 343 aa, Mouse
MTRSPALLLLLLGALPSAEAARDDISPGKESPGPGGSSGGQEDPASQQWERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 100 - DNA, 1236 bp, Mouse
atgacgcggagccccgcgctgctgctgctgctattgggggccctcccgtcggctgaggcggcgcgagcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 101 - protein, 411 aa, Mouse
MTRSPALLLLLLGALPSAEAARARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 102 - DNA, 870 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 103 - protein, 289 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 104 - DNA, 678 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 105 - protein, 225 aa, Mouse
MTRSPALLLLLLGALPSAEAARDDISPGKESPGPGGSSGGQEDPASQQWDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 106 - DNA, 882 bp, Mouse
atgacgcggagccccgcgctgctgctgctgctattgggggccctcccgtcggctgaggcggcgcgagcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 107 - protein, 293 aa, Mouse
MTRSPALLLLLLGALPSAEAARARPRFTQPSKMRRRVIARPVGSSVRLKCVASGHPRPDIMWMKDDQTLTHLEASEHRKKKWTLSLKNLKPEDSGKYTCRVSNKAGAINATYKVDVIHPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 108 - DNA, 951 bp, Mouse
atgacgcggagccccgcgctgctgctgctgctattgggggccctcccgtcggctgaggcggcgcgagagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 109 - protein, 316 aa, Mouse
MTRSPALLLLLLGALPSAEAARERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 110 - DNA, 597 bp, Mouse
atgacgcggagccccgcgctgctgctgctgctattgggggccctcccgtcggctgaggcggcgcgagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatttgggggccaaggcaacaggttgggagaattgagaacaatggaggaagagtatcttag

SEQ ID NO: 111 - protein, 198 aa, Mouse
MTRSPALLLLLLGALPSAEAARDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 112 - DNA, 1060 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 113 - protein, 353 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 114 - DNA, 706 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatcc

SEQ ID NO: 115 - protein, 235 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVI

SEQ ID NO: 116 - DNA, 775 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 117 - protein, 258 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 118 - DNA, 979 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 119 - protein, 326 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 120 - DNA, 787 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 121 - protein, 262 aa, Mouse
MTRSPALLLLLLGALPSAEAARDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 122 - DNA, 421 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtggg

SEQ ID NO: 123 - protein, 140 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQW

SEQ ID NO: 124 - DNA, 625 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatcc

SEQ ID NO: 125 - protein, 208 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMARPRFTQPSKMRRRVIARPVGSSVRLKCVASGHPRPDIMWMKDDQTLTHLEASEHRKKKWTLSLKNLKPEDSGKYTCRVSNKAGAINATYKVDVI

SEQ ID NO: 126 - DNA, 694 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 127 - protein, 231 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 128 - DNA, 433 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatcc

SEQ ID NO: 129 - protein, 144 aa, Mouse
MTRSPALLLLLLGALPSAEAARDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVI

SEQ ID NO: 130 - DNA, 502 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 131 - protein, 167 aa, Mouse
MTRSPALLLLLLGALPSAEAARDDISPGKESPGPGGSSGGQEDPASQQWERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 132 - DNA, 706 bp, Mouse
atgacgcggagccccgcgctgctgctgctgctattgggggccctcccgtcggctgaggcggcgcgagcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 133 - protein, 235 aa, Mouse
MTRSPALLLLLLGALPSAEAARARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 134 - DNA, 340 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatgg

SEQ ID NO: 135 - protein, 113 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIM

SEQ ID NO: 136 - DNA, 148 bp, Mouse
atgacgcggagccccgcgctgctgctgctgctattgggggccctcccgtcggctgaggcggcgcgagatgatattagtccagggaaggagagccctgggccaggtggttcttcggggggccaggaggacccagccagccagcagtggg

SEQ ID NO: 137 - protein, 49 aa, Mouse
MTRSPALLLLLLGALPSAEAARDDISPGKESPGPGGSSGGQEDPASQQW

SEQ ID NO: 138 - DNA, 352 bp, Mouse
atgacgcggagccccgcgctgctgctgctgctattgggggccctcccgtcggctgaggcggcgcgagcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatcc

SEQ ID NO: 139 - protein, 117 aa, Mouse
MTRSPALLLLLLGALPSAEAARARPRFTQPSKMRRRVIARPVGSSVRLKCVASGHPRPDIMWMKDDQTLTHLEASEHRKKKWTLSLKNLKPEDSGKYTCRVSNKAGAINATYKVDVI

SEQ ID NO: 140 - DNA, 421 bp, Mouse
atgacgcggagccccgcgctgctgctgctgctattgggggccctcccgtcggctgaggcggcgcgagagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccag

SEQ ID NO: 141 - protein, 140 aa, Mouse
MTRSPALLLLLLGALPSAEAARERTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLP

SEQ ID NO: 142 - DNA, 67 bp, Mouse
atgacgcggagccccgcgctgctgctgctgctattgggggccctcccgtcggctgaggcggcgcgag

SEQ ID NO: 143 - protein, 22 aa, Mouse
MTRSPALLLLLLGALPSAEAAR

SEQ ID NO: 144 - DNA, 1389 bp, Mouse
atgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggcggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacacctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatatgggggccaaggcaacaggttgggagaattgagaacaatggaggaagagtatcttag

SEQ ID NO: 145 - protein, 462 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 146 - protein, 5 aa, Artificial Sequence
  Made in a lab
GGGGS

SEQ ID NO: 147 - protein, 10 aa, Artificial Sequence
  Made in a lab
GGGGSGGGGS

SEQ ID NO: 148 - protein, 15 aa, Artificial Sequence
  Made in a lab
GGGGSGGGGSGGGGS

SEQ ID NO: 149 - protein, 20 aa, Artificial Sequence
  Made in a lab
GGGGSGGGGSGGGGSGGGGS

SEQ ID NO: 150 - DNA, 16 bp, Artificial Sequence
  Made in a lab
ggcttcggcagcctta

SEQ ID NO: 151 - DNA, 21 bp, Artificial Sequence
  Made in a lab
cattagcccagggaaggagag

SEQ ID NO: 152 - DNA, 27 bp, Artificial Sequence
  Made in a lab
caactacaccctcgtcgtgctggatga

SEQ ID NO: 153 - protein, 504 aa, Human; identical to SEQ ID NO: 17

SEQ ID NO: 154 - DNA, 3397 bp, Human
gccgccccgccccgagaccgggcccgggggcgcggggcggcgggatgcggcgcccggggcggcgatgaccgcggagcgcacgccgcgggcccggccctgaccccgccgcccgcccgctgagccccccgccgaggtccggacaggccgagatgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagcctgagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgccagggccacctgtggcctcctcgtcctcggccactatcggcatcccagccggcgctgtcttcatcctgggcaccctgctcctgtggctttgccaggcccagaagaagccgtgcacccccgcgcctgcccctcccctgcctgggcaccgcccgccggggacggcccgcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctagacggcaccgtatctgcagtgggcacgggggggccggccagacaggcagactgggaggatggaggacggagctgcagacgaaggcaggggacccatggcgaggaggaatggccagcaccccaggcagtctgtgtgtgaggcatagcccctggacacacacacacagacacacacactacctggatgcatgtatgcacacacatgcgcgcacacgtgctccctgaaggcacacgtacgcacacgcacatgcacagatatgccgcctgggcacacagataagctgcccaaatgcacgcacacgcacagagacatgccagaacatacaaggacatgctgcctgaacatacacacgcacacccatgcgcagatgtgctgcctggacacacacacacacacggatatgctgtctggacgcacacacgtgcagatatggtatccggacacacacgtgcacagatatgctgcctggacacacagataatgctgccttgacacacacatgcacggatattgcctggacacacacacacacacacgcgtgcacagatatgctgtctggacacgcacacacatgcagatatgctgcctggacacacacttccagacacacgtgcacaggcgcagatatgctgcctggacacacgcagatatgctgtctagtcacacacacacgcagacatgctgtccggacacacacacgcatgcacagatatgctgtccggacacacacacgcacgcagatatgctgcctggacacacacacagataatgctgcctcaacactcacacacgtgcagatattgcctggacacacacatgtgcacagatatgctgtctggacatgcacacacgtgcagatatgctgtccggatacacacgcacgcacacatgcagatatgctgcctgggcacacacttccggacacacatgcacacacaggtgcagatatgctgcctggacacacacacagataatgctgcctcaacactcacacacgtgcagatattgcctggacacacacatgtgcacagatatgctgtctggacatgcacacacgtgcagatatgctgtccggatacacacgcacgcacacatgcagatatgctgcctgggcacacacttccggacacacatgcacacacaggtgcagatatgctgcctggacacacgcagactgacgtgcttttgggagggtgtgccgtgaagcctgcagtacgtgtgccgtgaggctcatagttgatgagggactttccctgctccaccgtcactcccccaactctgcccgcctctgtccccgcctcagtccccgcctccatccccgcctctgtcccctggccttggcggctatttttgccacctgccttgggtgcccaggagtcccctactgctgtgggctggggttgggggcacagcagccccaagcctgagaggctggagcccatggctagtggctcatccccagtgcattctccccctgacacagagaaggggccttggtatttatatttaagaaatgaagataatattaataatgatggaaggaagactgggttgcagggactgtggtctctcctggggcccgggacccgcctggtctttcagccatgctgatgaccacaccccgtccaggccagacaccaccccccaccccactgtcgtggtggccccagatctctgtaattttatgtagagtttgagctgaagccccgtatatttaatttattttgttaaacacaaaaaaaaaaaaaaaaaaaa

SEQ ID NO: 155 - DNA, 27 bp, Artificial Sequence
  Made in a lab
gaggaacagatttctgatcatactttc

SEQ ID NO: 156 - DNA, 22 bp, Artificial Sequence
  Made in a lab
catttgtttgttacccttgccc

SEQ ID NO: 157 - DNA, 25 bp, Artificial Sequence
  Made in a lab
tgtgtgactcacggatgacttccac

SEQ ID NO: 158 - DNA, 23 bp, Artificial Sequence
  Made in a lab
cctctttgatgcggagagcttgc

SEQ ID NO: 159 - DNA, 23 bp, Artificial Sequence
  Made in a lab
caatatcaactacaccctcatcg

SEQ ID NO: 160 - DNA, 23 bp, Artificial Sequence
  Made in a lab
cttgacatcactgcgtactttgc

SEQ ID NO: 161 - DNA, 24 bp, Artificial Sequence
  Made in a lab
caaaatgagaaagcgtgtgattgc

SEQ ID NO: 162 - DNA, 22 bp, Artificial Sequence
  Made in a lab
cttccggcgtgttggagaattc

SEQ ID NO: 163 - DNA, 24 bp, Artificial Sequence
  Made in a lab
cagttctcacgtctgctacttgca